SEQUENCE LISTING (20 sequences)

SEQ ID NO: 1 - protein, 133 aa, Homo sapiens [sequence omitted]

SEQ ID NO: 2 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 3 - protein, 609 aa, Homo sapiens [sequence omitted]

SEQ ID NO: 4 - protein, 585 aa, Homo sapiens [sequence omitted]

SEQ ID NO: 5 - protein, 752 aa, Artificial Sequence
  hAlb-hIL2
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGGGGSGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 752 aa, Artificial Sequence
  hAlb-hIL2_A4s8
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGGGGSGGAPTSSSTKKTQLQLEHLLLDLQMILNGINNYKNPKLTRMLTFEFYMPKKATELKHLQCLEKELKPLEEVLNLAHSKNFHFEPRDVISNINVFVLELKGSETTFMCEYADETATIVEFLNRWITFCQSIISTLT

SEQ ID NO: 7 - protein, 772 aa, Artificial Sequence
  hIL7 hAlb
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGSGGGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 26 aa, Artificial Sequence
  Secretory pptide
MRVMAPRTLILLLSGALALTETWAGS

SEQ ID NO: 9 - protein, 55 aa, Artificial Sequence
  MITD
IVGIVAGLAVLAVVVIGAVVATVMCRRKSSGGKGGSYSQAASSDSAQGSDVSLTA

SEQ ID NO: 10 - protein, 65 aa, Artificial Sequence
  P2P16
KKQYIKANSKFIGITELKKLGGGKRGGGKKMTNSVDDALINSTKIYSYFPSVISKVNQGAQGKKL

SEQ ID NO: 11 - protein, 10 aa, Artificial Sequence
  Linker
GGSGGGGSGG

SEQ ID NO: 12 - protein, 14 aa, Artificial Sequence
  Linker
GSSGGGGSPGGGSS

SEQ ID NO: 13 - RNA, 47 nt, Artificial Sequence
  5′-UTR
aacuaguauucuucugguccccacagacucagagagaacccgccacc

SEQ ID NO: 14 - RNA, 278 nt, Artificial Sequence
  3′-UTR
cugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacc

SEQ ID NO: 15 - RNA, 110 nt, Artificial Sequence
  A30L70
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 16 - protein, 15 aa, Artificial Sequence
  P2
QYIKANSKFIGITEL

SEQ ID NO: 17 - protein, 32 aa, Artificial Sequence
  P16
MTNSVDDALINSTKIYSYFPSVISKVNQGAQG

SEQ ID NO: 18 - RNA, 2741 nt, Artificial Sequence
  hAlb-hIL2
agacgaacuaguauucuucugguccccacagacucagagagaacccgccaccaugaaguggguaaccuuuauuucccuucuuuuucucuuuagcucggcuuauuccagggguguguuucgucgagaugcacacaagagugagguugcucaucgcuuuaaagauuugggagaagaaaauuucaaagccuugguguugauugccuuugcucaguaucuucagcaguguccauuugaagaucauguaaaauuagugaaugaaguaacugaauuugcaaaaacauguguugcugaugagucagcugaaaauugugacaaaucacuucauacccuuuuuggagacaaauuaugcacaguugcaacacuucgugaaaccuauggugaaauggcugacugcugugcaaaacaagaaccugagagaaaugaaugcuucuugcaacacaaagaugacaacccaaaccucccccgauuggugagaccagagguugaugugaugugcacugcuuuucaugacaaugaagaaacauuuuugaaaaaauacuuauaugaaauugccagaagacauccuuacuuuuaugccccggaacuccuuuucuuugcuaaaagguauaaagcugcuuuuacagaauguugccaagcugcugauaaagcugccugccuguugccaaagcucgaugaacuucgggaugaagggaaggcuucgucugccaaacagagacucaagugugccagucuccaaaaauuuggagaaagagcuuucaaagcaugggcaguagcucgccugagccagagauuucccaaagcugaguuugcagaaguuuccaaguuagugacagaucuuaccaaaguccacacggaaugcugccauggagaucugcuugaaugugcugaugacagggcggaccuugccaaguauaucugugaaaaucaagauucgaucuccaguaaacugaaggaaugcugugaaaaaccacuguuggaaaaaucccacugcauugccgaaguggaaaaugaugagaugccugcugacuugccuucauuagcugcugauuuuguugaaaguaaggauguuugcaaaaacuaugcugaggcaaaggaugucuuccugggcauguuuuuguaugaauaugcaagaaggcauccugauuacucugucgugcugcugcugagacuugccaagacauaugaaaccacucuagagaagugcugugccgcugcagauccucaugaaugcuaugccaaaguguucgaugaauuuaaaccucuuguggaggagccucagaauuuaaucaaacaaaauugugagcuuuuugagcagcuuggagaguacaaauuccagaaugcgcuauuaguucguuacaccaagaaaguaccccaagugucaacuccaacucuuguagaggucucaagaaaccuaggaaaagugggcagcaaauguuguaaacauccugaagcaaaaagaaugcccugugcagaagacuaucuauccgugguccugaaccaguuauguguguugcaugagaaaacgccaguaagugacagagucaccaaaugcugcacagaauccuuggugaacaggcgaccaugcuuuucagcucuggaagucgaugaaacauacguucccaaagaguuuaaugcugaaacauucaccuuccaugcagauauaugcacacuuucugagaaggagagacaaaucaagaaacaaacugcacuuguugagcuggugaaacacaagcccaaggcaacaaaagagcaacugaaagcuguuauggaugauuucgcagcuuuuguagagaagugcugcaaggcugacgauaaggagaccugcuuugccgaggaggguaaaaaacuuguugcugcaagucaagcugccuuaggcuuaggcggcucuggaggaggcggcuccggaggcgcuccaacaucuucuucaacaaagaaaacacagcuucagcuugaacaccuucuucuugaucuucagaugauucugaauggaaucaacaauuacaaaaauccaaaacugacaagaaugcugacauuuaaauuuuacaugccaaagaaagcaacagaacugaaacaccuucagugccuugaagaagaacugaaaccucuggaagaagugcugaaucuggcucagagcaaaaauuuucaccugagaccaagagaucugaucagcaacaucaaugugauugugcuggaacugaaaggaucugaaacaacauucaugugugaauaugcugaugaaacagcaacaauuguggaauuucugaacagauggauuacauuuugccagucaaucauuucaacacugacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 19 - RNA, 2741 nt, Artificial Sequence
  hAlb-hIL2_A4s8
agacgaacuaguauucuucugguccccacagacucagagagaacccgccaccaugaaguggguaaccuuuauuucccuucuuuuucucuuuagcucggcuuauuccagggguguguuucgucgagaugcacacaagagugagguugcucaucgcuuuaaagauuugggagaagaaaauuucaaagccuugguguugauugccuuugcucaguaucuucagcaguguccauuugaagaucauguaaaauuagugaaugaaguaacugaauuugcaaaaacauguguugcugaugagucagcugaaaauugugacaaaucacuucauacccuuuuuggagacaaauuaugcacaguugcaacacuucgugaaaccuauggugaaauggcugacugcugugcaaaacaagaaccugagagaaaugaaugcuucuugcaacacaaagaugacaacccaaaccucccccgauuggugagaccagagguugaugugaugugcacugcuuuucaugacaaugaagaaacauuuuugaaaaaauacuuauaugaaauugccagaagacauccuuacuuuuaugccccggaacuccuuuucuuugcuaaaagguauaaagcugcuuuuacagaauguugccaagcugcugauaaagcugccugccuguugccaaagcucgaugaacuucgggaugaagggaaggcuucgucugccaaacagagacucaagugugccagucuccaaaaauuuggagaaagagcuuucaaagcaugggcaguagcucgccugagccagagauuucccaaagcugaguuugcagaaguuuccaaguuagugacagaucuuaccaaaguccacacggaaugcugccauggagaucugcuugaaugugcugaugacagggcggaccuugccaaguauaucugugaaaaucaagauucgaucuccaguaaacugaaggaaugcugugaaaaaccacuguuggaaaaaucccacugcauugccgaaguggaaaaugaugagaugccugcugacuugccuucauuagcugcugauuuuguugaaaguaaggauguuugcaaaaacuaugcugaggcaaaggaugucuuccugggcauguuuuuguaugaauaugcaagaaggcauccugauuacucugucgugcugcugcugagacuugccaagacauaugaaaccacucuagagaagugcugugccgcugcagauccucaugaaugcuaugccaaaguguucgaugaauuuaaaccucuuguggaggagccucagaauuuaaucaaacaaaauugugagcuuuuugagcagcuuggagaguacaaauuccagaaugcgcuauuaguucguuacaccaagaaaguaccccaagugucaacuccaacucuuguagaggucucaagaaaccuaggaaaagugggcagcaaauguuguaaacauccugaagcaaaaagaaugcccugugcagaagacuaucuauccgugguccugaaccaguuauguguguugcaugagaaaacgccaguaagugacagagucaccaaaugcugcacagaauccuuggugaacaggcgaccaugcuuuucagcucuggaagucgaugaaacauacguucccaaagaguuuaaugcugaaacauucaccuuccaugcagauauaugcacacuuucugagaaggagagacaaaucaagaaacaaacugcacuuguugagcuggugaaacacaagcccaaggcaacaaaagagcaacugaaagcuguuauggaugauuucgcagcuuuuguagagaagugcugcaaggcugacgauaaggagaccugcuuugccgaggaggguaaaaaacuuguugcugcaagucaagcugccuuaggcuuaggcggcucuggaggaggcggcuccggaggcgcuccaacaucuucuucaacaaagaaaacacagcuucagcuugaacaccuucuucuugaucuucagaugauucugaauggaaucaacaauuacaaaaauccaaaacugacaagaaugcugacauuugaauuuuacaugccaaagaaagcaacagaacugaaacaccuucagugccuugaaaaagaacugaaaccucuggaagaagugcugaaucuggcucacagcaaaaauuuucacuuugaaccaagagaugugaucagcaacaucaauguguuugugcuggaacugaaaggaucugaaacaacauucaugugugaauaugcugaugaaacagcaacaauuguggaauuucugaacagauggauuacauuuugccagucaaucauuucaacacugacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 20 - RNA, 2801 nt, Artificial Sequence
  hIL7 hAlb
agacgaacuaguauucuucugguccccacagacucagagagaacccgccaccauguuccauguuucuuuuagguauaucuuuggacuuccuccccugauccuuguucuguugccaguagcaucaucugauugugauauugaagguaaagauggcaaacaauaugagaguguucuaauggucagcaucgaucaauuauuggacagcaugaaagaaauugguagcaauugccugaauaaugaauuuaacuuuuuuaaaagacauaucugugaugcuaauaaggaagguauguuuuuauuccgugcugcucgcaaguugaggcaauuucuuaaaaugaauagcacuggugauuuugaucuccacuuauuaaaaguuucagaaggcacaacaauacuguugaacugcacuggccagguuaaaggaagaaaaccagcugcccugggugaagcccaaccaacaaagaguuuggaagaaaauaaaucuuuaaaggaacagaaaaaacugaaugacuuguguuuccuaaagagacuauuacaagagauaaaaacuuguuggaauaaaauuuugaugggcacuaaagaacacggcggcucuggaggaggcggcuccggaggcgaugcacacaagagugagguugcucaucgcuuuaaagauuugggagaagaaaauuucaaagccuugguguugauugccuuugcucaguaucuucagcaguguccauuugaagaucauguaaaauuagugaaugaaguaacugaauuugcaaaaacauguguugcugaugagucagcugaaaauugugacaaaucacuucauacccuuuuuggagacaaauuaugcacaguugcaacacuucgugaaaccuauggugaaauggcugacugcugugcaaaacaagaaccugagagaaaugaaugcuucuugcaacacaaagaugacaacccaaaccucccccgauuggugagaccagagguugaugugaugugcacugcuuuucaugacaaugaagaaacauuuuugaaaaaauacuuauaugaaauugccagaagacauccuuacuuuuaugccccggaacuccuuuucuuugcuaaaagguauaaagcugcuuuuacagaauguugccaagcugcugauaaagcugccugccuguugccaaagcucgaugaacuucgggaugaagggaaggcuucgucugccaaacagagacucaagugugccagucuccaaaaauuuggagaaagagcuuucaaagcaugggcaguagcucgccugagccagagauuucccaaagcugaguuugcagaaguuuccaaguuagugacagaucuuaccaaaguccacacggaaugcugccauggagaucugcuugaaugugcugaugacagggcggaccuugccaaguauaucugugaaaaucaagauucgaucuccaguaaacugaaggaaugcugugaaaaaccacuguuggaaaaaucccacugcauugccgaaguggaaaaugaugagaugccugcugacuugccuucauuagcugcugauuuuguugaaaguaaggauguuugcaaaaacuaugcugaggcaaaggaugucuuccugggcauguuuuuguaugaauaugcaagaaggcauccugauuacucugucgugcugcugcugagacuugccaagacauaugaaaccacucuagagaagugcugugccgcugcagauccucaugaaugcuaugccaaaguguucgaugaauuuaaaccucuuguggaggagccucagaauuuaaucaaacaaaauugugagcuuuuugagcagcuuggagaguacaaauuccagaaugcgcuauuaguucguuacaccaagaaaguaccccaagugucaacuccaacucuuguagaggucucaagaaaccuaggaaaagugggcagcaaauguuguaaacauccugaagcaaaaagaaugcccugugcagaagacuaucuauccgugguccugaaccaguuauguguguugcaugagaaaacgccaguaagugacagagucaccaaaugcugcacagaauccuuggugaacaggcgaccaugcuuuucagcucuggaagucgaugaaacauacguucccaaagaguuuaaugcugaaacauucaccuuccaugcagauauaugcacacuuucugagaaggagagacaaaucaagaaacaaacugcacuuguugagcuggugaaacacaagcccaaggcaacaaaagagcaacugaaagcuguuauggaugauuucgcagcuuuuguagagaagugcugcaaggcugacgauaaggagaccugcuuugccgaggaggguaaaaaacuuguugcugcaagucaagcugccuuaggcuuaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0390462 A1
(43) Pub. Date: Nov. 28, 2024

(54) Treatment Schedule for Cytokine Proteins

(71) Applicant: BIONTECH SE, Mainz (DE)

(72) Inventors: Mathias VORMEHR, Mainz (DE); Lena Mareen KRANZ, Mainz (DE)

(21) Appl. No.: 18/268,594

(22) PCT Filed: Dec. 20, 2021
(86) PCT No.: PCT/EP2021/086761

§ 371 Date: Jun. 20, 2023

Related U.S. Application Data
(63) Continuation of application No. PCT/EP2020/087467, filed on Dec. 21, 2020.

(51) Int. Cl. A61K 38/20

(52) U.S. Cl. CPC A61K 38/2013

(57) ABSTRACT

This disclosure relates to the field of therapeutic RNA, in particular to treat cancer. Disclosed herein are compositions, uses, and methods for reducing an unwanted response or reaction, or both, in a subject, to RNA encoding an amino acid sequence comprising a cytokine protein.

This application is a U.S. national phase application under 35 U.S.C. 371 of International Patent Application No. PCT/EP2021/086761 filed on Dec. 20, 2021, which claims the benefit of International Patent Application No. PCT/EP2020/087467 filed on Dec. 21, 2020, the disclosures of each of which are incorporated by reference herein in their entirety.

### REFERENCE TO AN ELECTRONIC SEQUENCE LISTING

A computer readable form of the Sequence Listing is filed with this application by electronic submission and is hereby incorporated by reference in its entirety. The Sequence Listing is contained in the text file created on Dec. 20, 2021, having the name “674-367_PCT2_SeqProt_ST25.txt” and is 47,432 bytes in size.

This disclosure relates to the field of therapeutic RNA, in particular to treat cancer. Disclosed herein are compositions, uses, and methods for reducing an unwanted response or reaction, or both, in a subject, to RNA encoding an amino acid sequence comprising a cytokine protein.

### BACKGROUND

The immune system plays a key role in preventing and combating cancer (Mittal et al., Curr. Opinion Immunol. (2014)). Effective antitumor immunity requires concerted action of different immune cell subsets. T cells and natural killer (NK) cells are important mediators of antitumor immune responses. CD8⁺ T cells and NK cells can directly lyse tumor cells. CD4⁺ T cells promote the influx of immune subsets into the tumor, license dendritic cells (DCs) for the priming of antitumor CD8⁺ T cell responses and induce IFNγ-mediated inhibition of tumor cell growth. IFNγ and type I interferons induced during inflammation increase the presentation of tumor antigens in the context of MHC class I molecules, thereby promoting tumor cell recognition by CD8⁺ T cells.

With cures remaining scarce in patients with advanced solid tumors, there is an urgent unmet medical need for more effective and less toxic regimens. Immunostimulatory cytokines are essential for efficacious antitumor immune responses, and cytokine-based treatments represent a promising therapeutic avenue.

Interleukin-2 (IL2) is a potent immune-stimulatory cytokine acting on diverse cells of the immune system, including T cells and NK cells. IL2 is known to support the differentiation, proliferation, survival and effector functions of T cells and NK cells (Blattman, J. N. et al. Nat. Med. 9, 540-7 (2003)). This cytokine is produced primarily by T cells upon activation (Spolski et al., Nat. Rev. Immunol. (2018)).

IL2 preferentially stimulates cells expressing the high-affinity IL2 receptor (IL2Rαβγ) consisting of CD25 (IL2Rα), CD122 (IL2Rβ) and CD132 (IL2Rγ). The high-affinity IL2Rαβγ complex is constitutively expressed on immune suppressive regulatory T (T_{reg}) cells and is temporarily present on activated CD4⁺ and CD8⁺ T cells. This highlights the dual role of this cytokine in antitumor immune responses, as IL2 not only enhances the function of antitumor effector T cells and NK cells, but also stimulates the proliferation and immune inhibitory function of T_{reg} cells (Spolski et al., Nat. Rev. Immunol. (2018)).

Recombinant human IL2 (rhIL2), aldesleukin, is the first approved cancer immunotherapy. Aldesleukin has long been used in the treatment of late-stage malignant melanoma and renal cell cancer (Kammula et al. 1998). Most patients with complete responses after rhIL2 therapy remain relapse-free for more than 25 years after the initial treatment, yet the overall response rates are low (Klapper et al. 2008, Rosenberg et al. 1998).

There are several factors that limit the clinical efficacy of IL2. Firstly, due to its short plasma half-life, rhIL2 has to be administered frequently at high doses (Jiang et al., Oncoimmunol. (2016)). As the majority of naïve and memory T cells as well as NK cells only express the intermediate-affinity receptor IL2Rβγ (Stauber et al., PNAS (2006); Walsh, Immunol. Rev. (2012)), high doses of IL2 are needed to stimulate these cells and thereby achieve optimal antitumor immunity.

High-dose rhIL2 treatment can significantly increase overall survival in a subset of cancer patients (McDermott et al., J. Clin. Oncol. (2005)). Unfortunately, such regimens often cause severe adverse effects, such as vascular leak syndrome (VLS), leading to treatment-related mortality of up to 4% (McDermott et al., J. Clin. Oncol. (2005); Rosenberg, S. A. et al. N. Engl. J. Med. 316, 889-97 (1987)). The exact cause of VLS is only partially understood. It is believed that pro-inflammatory cytokines (such as IFNγ) from IL2-activated NK cells play an essential role (Assier et al., 2004). The overall contribution of NK cells to IL2-mediated toxic effects is well-documented (Gately, J. Immunol. (1988), Peace et al., J. Exp. Med. (1989)).

Another inherent disadvantage of rhIL2 is its ability to stimulate and expand T_{reg} cells in cancer patients. T_{reg} cells can suppress the function of antitumor effector T cells and NK cells (Sim et al., J. Clin. Invest. (2014); Todd et al., PLoS Med. (2016)), which has been linked to reduced overall survival and represents a major hurdle for the development of cancer immunotherapies based on rhIL2 (Ahmadzadeh & Rosenberg, Blood (2006)).

As rhIL2 has not shown robust clinical efficacy in cancer (Jiang et al., Oncoimmunol. (2016)), alternative approaches are necessary. We are developing the RiboCytokine platform technology (WO2019154985A1), which addresses the limitations of recombinant cytokines. RiboCytokine RNA translation into the encoded cytokines by the patient's liver cells ensures steady release of the active drug into the circulation, and fusion of the cytokine to human serum albumin (hAlb) mediates prolonged serum half-life and enrichment in secondary lymphoid organs and the tumor. This favorable pharmacokinetic profile of RiboCytokines contrasts with that of their recombinant counterparts, reduces the probability of severe toxicities associated with high cytokine concentrations in the blood, and helps avoid frequent dosing.

To specifically address the shortcomings of the pharmacodynamics of recombinant rhIL2, we have previously engineered pharmacologically optimized RNA encoding hAlb-hIL2_A4s8—a human IL2 variant fused to human serum albumin. This IL2 variant harbors mutations reducing its affinity for IL2Ra and increasing its binding to IL2Rβ.

In mice, injection of lipid nanoparticle (LNP)-formulated mRNA encoding hAlb-hIL2_A4s8 results in tumor growth inhibition and improved survival. Treatment with hAlb-hIL2_A4s8 reinforces CD8⁺ T cell antitumor responses while avoiding stimulation and expansion of T_{reg} cells.

These unique therapeutic and pharmacological properties of hAlb-hIL2_A4s8 call for developing treatment protocols with optimally balanced safety and efficacy. The present disclosure describes clinically relevant treatment regimens that enhance the tolerability of the RNA-encoded IL2-variant hAlb-hIL2_A4s8 and allow for expansion of both CD8⁺ T cells and NK cells. In addition to IL2 and IL2 variants, these findings are of relevance for any type of immunotherapy that leads to an expansion of NK cells (e.g. IL-15 and variants thereof, as well as type I interferon inducers).

### SUMMARY

The present invention generally embraces the immunotherapeutic treatment of a subject comprising the administration of RNA encoding an amino acid sequence comprising a cytokine protein, i.e., RNA encoding an amino acid sequence comprising a cytokine, a functional variant thereof, or a functional fragment of the cytokine or the functional variant (immunostimulant RNA). The amino acid sequence comprising a cytokine, a functional variant thereof, or a functional fragment of the cytokine or the functional variant is also referred to herein as “cytokine immunostimulant” or simply “immunostimulant”. In one embodiment, the RNA is administered for maintaining and/or stimulating T cells which are specific for a disease-associated antigen, e.g., an antigen expressed by a tumor.

In one embodiment, the present invention may further involve the administration of a vaccine to the subject, e.g., using RNA encoding one or more antigenic epitopes for stimulating T cells specific for the antigenic epitopes (vaccine RNA).

In order to reduce, i.e., lower or mitigate, the unwanted response or reaction, or both, caused by the expressed cytokine, functional variant, or functional fragment in the subject, the present invention provides for the administration of the immunostimulant RNA in a schedule, wherein a first dose of said RNA and a second dose of said RNA are administered, and wherein the dosages and time periods of administration of said first and second doses are selected such that the level of unwanted response or reaction is reduced in said subject. In one embodiment, the immunostimulant RNA and the encoded immunostimulant, respectively, are such that they would cause an unwanted response or reaction, in particular if not administered according to the schedule described herein, for example if the immunostimulant RNA is administered in a dose higher than the first dose (e.g., a second dose described herein), e.g., a therapeutically effective dose or target dose, without administering a lower priming dose (e.g., a first dose described herein). In one embodiment, the unwanted response or reaction involves NK cells and may comprise one or more selected from the group consisting of increase in NK cell number, fever, malaise, reduction of body weight, increase in activity of liver enzymes, capillary leak syndrome, hypotension and edema. In one embodiment, the liver enzymes comprise one or more selected from the group consisting of alanine-aminotransferase (ALAT), aspartate-aminotransferase (ASAT), and lactate-dehydrogenase (LDH).

In one embodiment, the immunostimulant RNA comprises or consists of RNA encoding an amino acid sequence comprising IL2, a functional variant thereof, or a functional fragment of the IL2 or the functional variant, e.g., RNA encoding an amino acid sequence comprising human IL2 (hIL2), a functional variant thereof, or a functional fragment of the hIL2 or the functional variant thereof. The immunostimulant RNA may comprise further RNA that encodes T cell stimulatory molecules and thus, may further comprise, for example, RNA encoding an amino acid sequence comprising IL7, a functional variant thereof, or a functional fragment of the IL7 or the functional variant thereof. In one embodiment, the immunostimulant, e.g., the IL protein, a functional variant thereof, or a functional fragment of the IL protein or the functional variant thereof, is fused, either directly or through a linker, to a pharmacokinetic modifying group. For example, the hIL, a functional variant thereof, or a functional fragment of the hIL or the functional variant thereof, may be fused, either directly or through a linker, to human albumin (hAlb), a functional variant thereof, or a functional fragment of the hAlb or the functional variant thereof.

In one embodiment, the RNA encoding one or more antigenic epitopes comprises one molecular species of RNA molecules, e.g., encoding a target antigen or a polyepitopic polypeptide, or RNA molecules of different molecular species, e.g., RNA molecules of 2, 3, 4, 5 or even more different molecular species. In one embodiment, such RNA molecules may encode one antigenic epitope or more than one, e.g., 2, 3, 4, 5, 6, 7, 8, 9, 10, 11, 12, 13, 14, 15 or even more, antigenic epitopes, e.g., different antigenic epitopes. The RNA encoding one or more antigenic epitopes is also designated herein as “vaccine RNA”. Such vaccine RNA encodes an amino acid sequence, i.e., a vaccine sequence, comprising one or more antigenic epitopes, i.e., an antigenic sequence. The one or more antigenic epitopes may be derived from one or more target antigens and, thus, may be suitable for inducing an immune response against the target antigen(s) or cells expressing the target antigen(s) in the subject. Vaccine RNA is administered to provide (following expression of the polynucleotide by appropriate target cells) epitopes for induction, i.e., stimulation, priming and/or expansion, of an immune response, in particular immune effector cells, which may be targeted to target antigen or a procession product thereof. In one embodiment, the immune response which is to be induced according to the present disclosure is a T cell-mediated immune response. In one embodiment, the immune response is an immune response against tumor or cancer cells, in particular tumor or cancer cells expressing a tumor antigen.

The compositions and methods described herein comprise as the active principle single-stranded RNA that may be translated into the respective protein upon entering cells of a recipient. In addition to wildtype or codon-optimized coding sequences, the RNA may contain one or more structural elements optimized for maximal efficacy of the RNA with respect to stability and translational efficiency (5′ cap, 5′ UTR, 3′ UTR, poly(A)-tail). As 5′-UTR sequence, the 5′-UTR sequence of the human alpha-globin mRNA, optionally with an optimized ‘Kozak sequence’ to increase translational efficiency may be used. As 3′-UTR sequence, a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used. These were identified by an ex vivo selection process for sequences that confer RNA stability and augment total protein expression (see WO 2017/060314, herein incorporated by reference). Furthermore, a poly(A)-tail measuring 110 nucleotides in length, consisting of a stretch of 30 adenosine residues, followed by a 10 nucleotide linker sequence (of random nucleotides) and another 70 adenosine residues may be used. This poly(A)-tail sequence was designed to enhance RNA stability and translational efficiency.

Furthermore, in the vaccine RNA, sec (secretory signal peptide) and/or MITD (MHC class I trafficking domain) may be fused to the epitope-encoding regions in a way that the respective elements are translated as N- or C-terminal tag, respectively. Fusion-protein tags derived from the sequence encoding the human MHC class I complex (HLA-B51, haplotype A2, B27/B51, Cw2/Cw3), have been shown to improve antigen processing and presentation. Sec may correspond to the 78 bp fragment coding for the secretory signal peptide, which guides translocation of the nascent polypeptide chain into the endoplasmatic reticulum. MITD may correspond to the transmembrane and cytoplasmic domain of the MHC class I molecule, also called MHC class I trafficking domain. Sequences coding for short linker peptides predominantly consisting of the amino acids glycine (G) and serine (S), as commonly used for fusion proteins may be used as GS/Linkers.

The antigenic epitopes may be administered in combination with helper epitopes to break immunological tolerance. The helper epitopes may be tetanus toxoid-derived, e.g., P2P16 amino acid sequences derived from the tetanus toxoid (TT) of Clostridium tetani. These sequences may support to overcome tolerance mechanisms by providing tumor-unspecific T cell help during priming. The tetanus toxoid heavy chain includes epitopes that can bind promiscuously to MHC class II alleles and induce CD4+ memory T cells in almost all tetanus vaccinated individuals. In addition, the combination of TT helper epitopes with tumor-associated antigens is known to improve the immune stimulation compared to the application of tumor-associated antigen alone by providing CD4+ mediated T cell help during priming. To reduce the risk of stimulating CD8+ T cells, two peptide sequences known to contain promiscuously binding helper epitopes may be used to ensure binding to as many MHC class II alleles as possible, e.g., P2 and P16.

In one embodiment, a vaccine sequence comprises an amino acid sequence which breaks immunological tolerance. In one embodiment, the amino acid sequence which breaks immunological tolerance comprises helper epitopes, preferably tetanus toxoid-derived helper epitopes. The amino acid sequence which breaks immunological tolerance may be fused to the C-terminus of the antigenic sequence, either directly or separated by a linker. Optionally, the amino acid sequence which breaks immunological tolerance may link the antigenic sequence and the MITD.

In one embodiment, the vaccine RNAs are applied together with RNA coding for a helper-epitope to boost the resulting immune response. This RNA coding for a helper-epitope may contain structural elements optimized for maximal efficacy of the RNA with respect to stability and translational efficiency (5′ cap, 5′ UTR, 3′ UTR, poly(A)-tail) described above.

The RNA, i.e., immunostimulant RNA and vaccine RNA, may be formulated in lipid particles to generate serum-stable formulations for intravenous (i.v.) administration. The immunostimulant RNA may be present in lipid nanoparticles (LNP). RNA-nanoparticles may target liver which results in an efficient expression of the encoded protein. In one embodiment, the immunostimulant RNA described herein is N1-methylpseudouridine modified, dsRNA-purified RNA which is formulated as lipid nanoparticles for intravenous administration. The vaccine RNA may be present in RNA-lipoplexes (LPX). RNA-lipoplexes may target antigen-presenting cells (APCs) in lymphoid organs which results in an efficient stimulation of the immune system. Different RNAs may be separately complexed with lipids to generate particulate formulations. In one embodiment, vaccine RNA is co-formulated as particles with an RNA encoding an amino acid sequence which breaks immunological tolerance.

In one aspect, provided herein is a method of reducing an unwanted response or reaction, or both, in a subject, to RNA encoding an amino acid sequence comprising a cytokine protein, said method comprising administering to a subject:
a first dose of said RNA;
a second dose of said RNA; and
wherein the dosages and time periods of administration of said first and second doses are selected such that the level of unwanted response or reaction is reduced in said subject.

In one embodiment, the amount of said RNA administered in said first dose is no more than 80%, 75%, 50%, 40%, 30%, 25%, 20%, 15%, 10% or 5% of the amount of said RNA administered in said second dose.

In one embodiment, the amount of said RNA administered in said first dose is no more than 200 μg, 150 μg, 100 μg, 90 μg, 80 μg, 70 μg, 60 μg, 50 μg, 40 μg, 30 μg, 20 μg, 10 μg, 5 μg, 4 μg, 3 μg, 2 μg, 1 μg, 0.5 μg, 0.4 μg, 0.3 μg, 0.2 μg, or 0.1 μg per kg body weight, and the second dose is greater than said first dose.

In one embodiment, the amount of said RNA administered in said second dose is greater than 20 μg, 30 μg, 40 μg, 50 μg, 60 μg, 70 μg, 80 μg, 90 μg, 100 μg, 150 μg, 200 μg, 250 μg, 300 μg, 350 μg, or 400 μg per kg body weight, and the second dose is greater than said first dose.

In one embodiment, more than 1, 2, 3, 4, 5, 6, 7, 14, or 21 days separate the completion of the administration of the first dose and the initiation of the administration of the second dose.

In one embodiment, no more than 56, 49, 42, 35, or 28 days separates the completion of the administration of the first dose and the initiation of the administration of the second dose.

In one embodiment, the method further comprises administering to the subject one or more additional doses of RNA encoding an amino acid sequence comprising a cytokine protein.

In one embodiment, said first and second doses are administered by intravenous, intraarterial, subcutaneous, intraperitoneal, intradermal or intramuscular injection or infusion.

In one embodiment, said first and second doses are administered intravenously.

In one embodiment, the unwanted response or reaction involves NK cells.

In one embodiment, the unwanted response or reaction comprises one or more selected from the group consisting of increase in NK cell number, fever, malaise, reduction of body weight, increase in activity of liver enzymes, capillary leak syndrome, hypotension and edema.

In one embodiment, the liver enzymes comprise one or more selected from the group consisting of alanine-aminotransferase (ALAT), aspartate-aminotransferase (ASAT), and lactate-dehydrogenase (LDH).

In one embodiment, the unwanted response or reaction occurs after administration of the second dose without administration of the first dose.

In one embodiment, the method further comprises evaluating the subject after administration of the first dose, the second dose, or both, for the presence of an unwanted response or reaction.

In one embodiment, said method does not cause a detectable unwanted response or reaction.

In one embodiment, said method results in a decrease in unwanted response or reaction.

In one embodiment, the method further comprises administering a vaccine to the subject.

In one embodiment, administering a vaccine to the subject comprises administering to the subject RNA encoding one or more antigenic epitopes.

In one embodiment, the epitopes are T cell epitopes.

In one embodiment, the amino acid sequence comprising a cytokine protein comprises an extended pharmacokinetic (PK) polypeptide.

In one embodiment, the extended PK polypeptide comprises a fusion protein.

In one embodiment, the fusion protein comprises the cytokine protein fused to a pharmacokinetic modifying group.

In one embodiment, the pharmacokinetic modifying group comprises albumin, a functional variant thereof, or a functional fragment of the albumin or the functional variant thereof.

In one embodiment, the pharmacokinetic modifying group comprises human albumin, a functional variant thereof, or a functional fragment of the human albumin or the functional variant thereof.

In one embodiment, the pharmacokinetic modifying group is fused to the N-terminus of the cytokine protein.

In one embodiment, the amino acid sequence comprising a cytokine protein comprises from N-terminus to C-terminus: N-pharmacokinetic modifying group-GS-linker-cytokine protein-C.

In one embodiment, the cytokine protein comprises an IL2 variant.

In one embodiment, the IL2 variant is a human IL2 variant.

In one embodiment, the human IL2 variant comprises a substitution variant of human IL2 or of a functional variant of human IL2.

In one embodiment, the substitution(s) enhance(s) the affinity for the βγ IL2 receptor complex (IL2Rβγ).

In one embodiment, the human IL2 or the functional variant thereof is substituted at at least position 80 (leucine), position 81 (arginine), position 85 (leucine) and position 92 (isoleucine) relative to wild type human IL2 and numbered in accordance with wild type human IL2.

In one embodiment, position 80 (leucine) is substituted by phenylalanine, position 81 (arginine) is substituted by glutamic acid, position 85 (leucine) is substituted by valine and position 92 (isoleucine) is substituted by phenylalanine relative to wild type human IL2 and numbered in accordance with wild type human IL2.

In one embodiment, the human IL2 or the functional variant thereof is further substituted at position 74 (glutamine) relative to wild type human IL2 and numbered in accordance with wild type human IL2.

In one embodiment, position 74 (glutamine) is substituted by histidine relative to wild type human IL2 and numbered in accordance with wild type human IL2.

In one embodiment, the substitution(s) reduce(s) the affinity for the alpha subunit of the αβγ IL2 receptor complex (IL2Rαβγ).

In one embodiment, the substitution(s) which reduce(s) the affinity for the alpha subunit of the αβγ IL2 receptor complex (IL2Rαβγ) reduce the affinity for IL2Rαβγ to a greater extent than for IL2Rβγ.

In one embodiment, the human IL2 or the functional variant thereof is substituted at at least position 43 (lysine) and position 61 (glutamic acid) relative to wild type human IL2 and numbered in accordance with wild type human IL2.

In one embodiment, position 43 (lysine) is substituted by glutamic acid and position 61 (glutamic acid) is substituted by lysine.

In one embodiment, the IL2 variant has a decreased ability to stimulate regulatory T cells compared to wild type human IL2.

In one embodiment, the IL2 variant has an increased ability to stimulate effector T cells compared to wild type human IL2.

In one embodiment, the cytokine protein comprises a mutein of human IL2 or of a functional variant of human IL2, wherein the human IL2 or the functional variant thereof is substituted at at least position 43 (lysine) by glutamic acid, position 61 (glutamic acid) by lysine, position 74 (glutamine) by histidine, position 80 (leucine) by phenylalanine, position 81 (arginine) by glutamic acid, position 85 (leucine) by valine and position 92 (isoleucine) by phenylalanine relative to wild type human IL2 and numbered in accordance with wild type human IL2.

In one embodiment, human IL2 has the amino acid sequence according to SEQ ID NO: 1.

In one embodiment, the amino acid sequence comprising a cytokine protein comprises the amino acid sequence according to SEQ ID NO: 6 (hAlb-hIL2_A4s8).

In one embodiment, the subject is a human.

In one embodiment, the immunostimulant RNA, i.e., the RNA encoding an amino acid sequence comprising a cytokine protein, e.g., RNA encoding an amino acid sequence comprising IL2, a functional variant thereof, or a functional fragment of the IL2 or the functional variant, comprises RNA encoding an amino acid sequence comprising human IL2 (hIL2), a functional variant thereof, or a functional fragment of the human IL2 or the functional variant thereof. In one embodiment, the amino acid sequence comprising human IL2, a functional variant thereof, or a functional fragment of the human IL2 or the functional variant thereof comprises human albumin (hAlb), a functional variant thereof, or a functional fragment of the hAlb or the functional variant thereof. In one embodiment, the hAlb, the functional variant thereof, or the functional fragment of the hAlb or the functional variant thereof is fused with the human IL2, the functional variant thereof, or the functional fragment of the human IL2 or the functional variant thereof. In one embodiment, the hAlb, the functional variant thereof, or the functional fragment of the hAlb or the functional variant thereof is fused to the N-terminus of the human IL2, the functional variant thereof, or the functional fragment of the human IL2 or the functional variant thereof.

In one embodiment, the immunostimulant RNA encodes an amino acid sequence comprising the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6. In one embodiment, the immunostimulant RNA encodes an amino acid sequence comprising the amino acid sequence of SEQ ID NO: 6.

In one embodiment, the immunostimulant is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment, the immunostimulant RNA comprises the 5′ cap m₂^{7,3′-O}Gppp(m₁^{2′-O})ApG. In one embodiment, the immunostimulant RNA is a modified RNA, in particular a stabilized mRNA. In one embodiment, the immunostimulant RNA comprises a modified nucleoside in place of at least one uridine. In one embodiment, the immunostimulant RNA comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, the immunostimulant RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In one embodiment, the immunostimulant RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

In one embodiment, the immunostimulant RNA comprises a poly-A sequence. In one embodiment, the poly-A sequence comprises at least 100 nucleotides. In one embodiment, the poly-A sequence comprises or consists of the nucleotide sequence of SEQ ID NO: 15.

In one embodiment, the immunostimulant comprises from N-terminus to C-terminus: N-hAlb-GS-linker-hIL2/hIL2 variant-C.

In one embodiment, the immunostimulant RNA is formulated as a liquid, formulated as a solid, or a combination thereof.

In one embodiment, the immunostimulant RNA is formulated for injection and/or is administered by injection.

In one embodiment, the immunostimulant RNA is formulated for intravenous administration and/or is administered by intravenous injection.

In one embodiment, the immunostimulant RNA is formulated or is to be formulated as lipid particles. In one embodiment, the RNA lipid particles are lipid nanoparticles (LNP). In one embodiment, the LNP particles comprise 3D-P-DMA, PEG₂₀₀₀-C-DMA, DSPC, and cholesterol. In one embodiment, the method described herein is a method for treating or preventing cancer. In one embodiment, the one or more antigenic epitopes are epitopes derived from tumor antigens.

In one embodiment, the vaccine RNA, i.e. RNA encoding one or more antigenic epitopes for stimulating T cells specific for the antigenic epitopes, encodes epitopes derived from one or more tumor antigens.

In one embodiment, the amino acid sequence encoded by the vaccine RNA, i.e., vaccine sequence, comprises an amino acid sequence enhancing antigen processing and/or presentation. In one embodiment, the amino acid sequence enhancing antigen processing and/or presentation comprises an amino acid sequence corresponding to the transmembrane and cytoplasmic domain of a MHC molecule, preferably a MHC class I molecule. In one embodiment, the amino acid sequence enhancing antigen processing and/or presentation comprises the amino acid sequence of SEQ ID NO: 9, or an amino acid sequence having at least 99%, 98%, 97%, 96%, 95%, 90%, 85%, or 80% identity to the amino acid sequence of SEQ ID NO: 9. In one embodiment, the amino acid sequence enhancing antigen processing and/or presentation further comprises an amino acid sequence coding for a secretory signal peptide.

In one embodiment, the secretory signal peptide comprises the amino acid sequence of SEQ ID NO: 8, or an amino acid sequence having at least 99%, 98%, 97%, 96%, 95%, 90%, 85%, or 80% identity to the amino acid sequence of SEQ ID NO: 8.

In one embodiment, the vaccine sequence comprises an amino acid sequence which breaks immunological tolerance and/or the vaccine RNA is co-administered with RNA encoding an amino acid sequence which breaks immunological tolerance. In one embodiment, the amino acid sequence which breaks immunological tolerance comprises helper epitopes, preferably tetanus toxoid-derived helper epitopes. In one embodiment, the amino acid sequence which breaks immunological tolerance comprises the amino acid sequence of SEQ ID NO: 10, or an amino acid sequence having at least 99%, 98%, 97%, 96%, 95%, 90%, 85%, or 80% identity to the amino acid sequence of SEQ ID NO: 10.

In one embodiment the vaccine sequence is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment, the vaccine RNA is a modified RNA, in particular a stabilized mRNA. In one embodiment, the RNA comprises a modified nucleoside in place of at least one uridine. In one embodiment, the RNA comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, the vaccine RNA comprises the 5′ cap m₂^{7,2′-O}Gppₛp(5′)G.

In one embodiment, the vaccine RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In one embodiment, the vaccine RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

In one embodiment, the vaccine RNA comprises a poly-A sequence. In one embodiment, the poly-A sequence comprises at least 100 nucleotides. In one embodiment, the poly-A sequence comprises or consists of the nucleotide sequence of SEQ ID NO: 15.

In one embodiment, the vaccine sequence, comprises from N-terminus to C-terminus: N-antigenic sequence-amino acid sequence which breaks immunological tolerance-amino acid sequence enhancing antigen processing and/or presentation-C.

In one embodiment, the vaccine RNA is formulated as a liquid, formulated as a solid, or a combination thereof.

In one embodiment, the vaccine RNA is formulated for injection and/or is administered by injection.

In one embodiment, the vaccine RNA is formulated for intravenous administration and/or is administered by intravenous administration.

In one embodiment, the vaccine RNA is formulated or is to be formulated as lipoplex particles.

In one embodiment, the RNA lipoplex particles are obtainable by mixing the RNA with liposomes.

In one aspect, provided herein is a kit comprising RNA as described herein; i.e., a first dose of RNA encoding an amino acid sequence comprising a cytokine protein and a second dose of RNA encoding an amino acid sequence comprising a cytokine protein, wherein the dosages of said first and second doses are selected such that upon administration of said first and second doses to a subject the level of unwanted response or reaction is reduced in said subject. The kit may comprise several doses, e.g., 2, 3, 4, 5 or even more, of the first dose and/or the second dose. The kit may further comprise RNA encoding one or more antigenic epitopes for stimulating T cells specific for the antigenic epitopes. Embodiments of these RNAs are as described herein, e.g., as described for the method described herein. In one embodiment, the different RNAs and/or different doses of RNA are in separate vials. In one embodiment, the kit comprises instructions for use of the RNAs in the method described herein. In one embodiment, the kit comprises instructions for use of the RNAs for treating or preventing cancer. In one embodiment, the one or more antigenic epitopes are derived from tumor antigens. In one aspect, provided herein is a kit described herein for pharmaceutical use. In one embodiment, the pharmaceutical use comprises a therapeutic or prophylactic treatment of a disease or disorder. In one embodiment, the therapeutic or prophylactic treatment of a disease or disorder comprises treating or preventing cancer.

In one aspect, provided herein is RNA described herein, i.e., a first dose of RNA encoding an amino acid sequence comprising a cytokine protein and a second dose of RNA encoding an amino acid sequence comprising a cytokine protein, wherein the dosages of said first and second doses are selected such that upon administration of said first and second doses to a subject the level of unwanted response or reaction is reduced in said subject, and optionally RNA encoding one or more antigenic epitopes for stimulating T cells specific for the antigenic epitopes, for use in a method described herein.

### BRIEF DESCRIPTION OF THE DRAWINGS

FIG. 1. Administration of hAlb-hIL2_A4s8, but not hAlb-hIL2, leads to toxicity in mice Naïve C57BL/6 mice (n=7 per group) were treated intravenously (i.v.) with either a triple combination of 5 μg RNA-LPX (encoding five prostate tumor antigens), 3 μg hAlb-hIL2_A4s8, and 3 μg hIL7-hAlb; 3 μg hAlb-hIL2_A4s8 and 3 μg hIL7-hAlb; 5 μg RNA-LPX and 3 μg hAlb-hIL2_A4s8; or 5 μg RNA-LPX and 3 μg hAlb-hIL2. All RiboCytokines were administered as LNP formulations throughout all the experiments described in the current disclosure (see Example 1). Mice treated with NaCl served as negative control. (A) Change of mouse body weight relative to the day of injection (day 0). Dotted line indicates no change. (B) Analysis of mouse serum for alanine aminotransferase (ALAT), aspartate aminotransferase (ASAT) and lactate dehydrogenase (LDH) activity as surrogates for liver and tissue damage three days after treatment. Dotted line, mean of the control group. Statistical significance was determined using two-way ANOVA and Dunnett's multiple comparisons test (A), and one-way ANOVA with Dunnett's multiple comparisons test (B). All analyses were two-tailed and carried out using GraphPad Prism 8. ns, not significant: P>0.05; **P≤0.01, ***P≤s0.001, ****P≤0.0001. Mean±SEM (A), and mean (B).

FIG. 2: hAlb-IL2_A4s8 triggers NK cell expansion, followed by contraction and unresponsiveness

(A) BALB/c mice (n=11 per group) were inoculated with 5×10⁵ CT26 tumor cells subcutaneously (s.c.) and treated i.v. weekly with 20 μg RNA-LPX vaccine encoding the tumor antigen gp70 on days 10, 17, 24 and 31. RiboCytokines hAlb-hIL2, hAlb-hIL2_A4s8 or hIL7-hAlb (3 μg each) were administered i.v. concomitantly with the RNA vaccine. The control group received RNA-LPX vaccine and 6 μg LNP-formulated RNA encoding hAlb only. Numbers of CD49b⁺CD19⁻CD4⁻CD8⁻ NK cells in the blood were determined by flow cytometry seven days after each treatment (days 17, 24, and 31). Dotted lines indicate treatment days, numbers at dotted lines indicate the number of administered doses of RNA-LPX plus RiboCytokine. (B) BALB/c mice (n=11 per group) were inoculated with 5×10⁵ CT26 tumor cells s.c. and treated i.v. weekly with 20 μg gp70 RNA-LPX and 3 μg RNA encoding hAlb-hIL2_A4s8, either individually or in combination, on days 10, 17, 24 and 31. The control group received an LPX-formulated RNA not encoding any antigen and 3 μg LNP-formulated hAlb-encoding RNA. Numbers of CD49b⁺CD19⁻CD4⁻CD8⁻ NK cells in the blood were determined by flow cytometry seven days after the first treatment (day 17). Dotted line, mean of the control group. Statistical significance was determined using two-way ANOVA followed by Dunnett's multiple comparisons test (A), and one-way ANOVA with Dunnett's multiple comparisons test (B). All analyses were two-tailed and carried out using GraphPad Prism 8. ns: P>0.05, ****P≤s0.0001. Mean±SEM (A), and mean (B).

FIG. 3. hAlb-hIL2_A4s8-associated toxicity is dependent on NK cells

C57BL/6 mice (n=7 per group) were treated i.v. with 3 μg hAlb-hIL2_A4s8, 3 μg hIL7-hAlb and 5 μg RNA-LPX on days 0, 7, 14 and 21 with or without prior NK cell depletion with polyclonal NK cell-depleting antibody (20 μL anti-asialo GM1 antibody administered via intraperitoneal injection) one day prior to RiboCytokine plus RNA-LPX treatment. Mice treated with NaCl served as negative control. (A) Change of mouse body weight relative to day 0. Dotted line indicates no change. (B) Analysis of mouse serum for ALAT, ASAT, and LDH activity as surrogates for liver and tissue damage three days after treatment. Dotted line, mean of NaCl group. Statistical significance was determined using two-way ANOVA followed by Dunnett's multiple comparisons test (A), and one-way ANOVA with Dunnett's multiple comparisons test (B). All analyses were two-tailed and carried out using GraphPad Prism 8. ns: P>0.05; **P≤0.01, ****P≤0.0001. Mean±SEM (A), and mean (B).

FIG. 4. Low-dose hIL2_A4s8 preconditioning promotes tolerability of subsequent treatments.

C57BL/6 mice were treated i.v. either with NaCl on day 0 (control; n=7); 3 μg hAlb-hIL2_A4s8, 3 μg hIL7-hAlb, and 5 μg RNA-LPX on day 0 (3 μg; n=7); 0.5 μg hAlb-hIL2_A4s8, 0.5 μg hIL7-hAlb, and 5 μg RNA-LPX on day 0, 7, 14, and 21 (0.5 μg; n=11); or 0.5 μg hAlb-hIL2_A4s8, 0.5 μg hIL7-hAlb, and 5 μg RNA-LPX on day 0, followed by 3 μg hAlb-hIL2_A4s8, 3 μg hIL7-hAlb, and 5 μg RNA-LPX on day 7, 14, and 21 (0.5 μg/3 μg; n=7). (A) Change of mouse body weight relative to day 0. Horizontal dotted line indicates no change. Vertical dotted lines indicate treatment days, numbers at dotted lines indicate the number of administered doses of RNA-LPX plus RiboCytokine. (B) Analysis of mouse serum for ALAT, ASAT, and LDH activity (day 3, n=7 per group; day 24, n=4). Dotted line, mean of the control group. Statistical significance was determined using one-way ANOVA and Dunnett's multiple comparisons test (B). All analyses were two-tailed and carried out using GraphPad Prism 8. ns: P>0.05, ****P≤0.0001. Mean±SEM (A), and mean (B).

FIG. 5. CD8⁺ T cells expand upon repeated hAlb-IL2_A4s8 treatments, whereas NK cells become refractory

C57BL/6 mice (n=5 per group) were treated i.v. either twice with 3 μg hAlb-hIL2A4s8, 3 weeks apart (day 0 and 21; every 3 weeks); once with 1.5 μg hAlb-hIL2_A4s8 (day 0) and once with 3 μg hAlb-hIL2_A4s8 (day 21; every 3 weeks dose escalation); once with 3 μg hAlb-hIL2_A4s8 (day 21; 3 weeks only); or three times with 3 μg hAlb-hIL2A4s8 (day 0, 7, 14; weekly). Frequencies of NK cells and CD8⁺ T cells were determined in the blood by flow cytometry on day 0, 7, 14, 21, 28, and 35. (A) Frequencies of NK cells among CD45⁺ cells. (B) Frequencies of CD8⁺ T cells among CD45⁺ cells. Dotted lines indicate treatment days, numbers at dotted lines indicate the number of administered doses of RiboCytokine. Mean±SEM.

FIG. 6. Low dose preconditioning increases the tolerability of hAlb-hIL2_A4s8 even in a three week dosing regime

C57BL/6 mice (n=5 per group) were treated i.v. either twice with 3 μg hAlb-hIL2A4s8, 3 weeks apart (day 0 and 21; every 3 weeks); once with 1.5 μg hAlb-hIL2_A4s8 (day 0) and once with 3 μg hAlb-hIL2_A4s8 (day 21; every 3 weeks dose escalation); once with 3 μg hAlb-hIL2_A4s8 (day 21; 3 weeks only); or three times with 3 μg hAlb-hIL2A4s8 (day 0, 7, 14; weekly). (A-E) Change of mouse body weight relative to (A) day 0 (day 0-3 shown), (B) day 7 (day 7-10 shown), (C) day 14 (day 14-17 shown), (D) day 21 (day 21-24 shown); and (E) day 0 (day 0-28 shown). Dotted lines indicate treatment days, numbers at dotted lines indicate number of treatments. Mean±SEM.

### DESCRIPTION OF THE SEQUENCES

The following table provides a listing of certain sequences referenced herein.

TABLE 1
DESCRIPTION OF THE SEQUENCES

| | | |
|---|---|---|
| SEQ | | |
| ID | | |
| NO: | Description | SEQUENCE |

hIL2 (mature)

| | | |
|---|---|---|
| 1 | amino | APTSSSTKKTQLQLEHLLLDLQMILNGINN |
| | acid | YKNPKLTRMLTFKFYMPKKATELKHLQCLE |
| | sequence | EELKPLEEVLNLAQSKNFHLRPRDLISNIN |
| | | VIVLELKGSETTFMCEYADETATIVEFLNR |
| | | WITFCQSIISTLT |

hIL7 (mature)

| | | |
|---|---|---|
| 2 | amino | DCDIEGKDGKQYESVLMVSIDQLLDSMKEI |
| | acid | GSNCLNNEFNFFKRHICDANKEGMFLFRAA |
| | sequence | RKLRQFLKMNSTGDFDLHLLKVSEGTTILL |
| | | NCTGQVKGRKPAALGEAQPTKSLEENKSLK |
| | | EQKKLNDLCFLKRLLQEIKTCWNKILMGTK |
| | | EH |

hAIb (non-mature)

| | | |
|---|---|---|
| 3 | amino | MKWVTFISLLFLFSSAYSRGVFRRDAHKSE |
| | acid | VAHRFKDLGEENFKALVLIAFAQYLQQCPF |
| | sequence | EDHVKLVNEVTEFAKTCVADESAENCDKSL |
| | | HTLFGDKLCTVATLRETYGEMADCCAKQEP |
| | | ERNECFLQHKDDNPNLPRLVRPEVDVMCTA |
| | | FHDNEETFLKKYLYEIARRHPYFYAPELLF |
| | | FAKRYKAAFTECCQAADKAACLLPKLDELR |
| | | DEGKASSAKQRLKCASLQKFGERAFKAWAV |
| | | ARLSQRFPKAEFAEVSKLVTDLTKVHTECC |
| | | HGDLLECADDRADLAKYICENQDSISSKLK |
| | | ECCEKPLLEKSHCIAEVENDEMPADLPSLA |
| | | ADFVESKDVCKNYAEAKDVFLGMFLYEYAR |
| | | RHPDYSVVLLLRLAKTYETTLEKCCAAADP |
| | | HECYAKVFDEFKPLVEEPQNLIKQNCELFE |
| | | QLGEYKFQNALLVRYTKKVPQVSTPTLVEV |
| | | SRNLGKVGSKCCKHPEAKRMPCAEDYLSVV |
| | | LNQLCVLHEKTPVSDRVTKCCTESLVNRRP |
| | | CFSALEVDETYVPKEFNAETFTFHADICTL |
| | | SEKERQIKKQTALVELVKHKPKATKEQLKA |
| | | VMDDFAAFVEKCCKADDKETCFAEEGKKLV |
| | | AASQAALGL |

hAIb (mature)

| | | |
|---|---|---|
| 4 | amino | DAHKSEVAHRFKDLGEENFKALVLIAFAQY |
| | acid | LQQCPFEDHVKLVNEVTEFAKTCVADESAE |
| | sequence | NCDKSLHTLFGDKLCTVATLRETYGEMADC |
| | | CAKQEPERNECFLQHKDDNPNLPRLVRPEV |
| | | DVMCTAFHDNEETFLKKYLYEIARRHPYFY |
| | | APELLFFAKRYKAAFTECCQAADKAACLLP |
| | | KLDELRDEGKASSAKQRLKCASLQKFGERA |
| | | FKAWAVARLSQRFPKAEFAEVSKLVTDLTK |
| | | VHTECCHGDLLECADDRADLAKYICENQDS |
| | | ISSKLKECCEKPLLEKSHCIAEVENDEMPA |
| | | DLPSLAADFVESKDVCKNYAEAKDVFLGMF |
| | | LYEYARRHPDYSVVLLLRLAKTYETTLEKC |
| | | CAAADPHECYAKVFDEFKPLVEEPQNLIKQ |
| | | NCELFEQLGEYKFQNALLVRYTKKVPQVST |
| | | PTLVEVSRNLGKVGSKCCKHPEAKRMPCAE |
| | | DYLSVVLNQLCVLHEKTPVSDRVTKCCTES |
| | | LVNRRPCFSALEVDETYVPKEFNAETFTFH |
| | | ADICTLSEKERQIKKQTALVELVKHKPKAT |
| | | KEQLKAVMDDFAAFVEKCCKADDKETCFAE |
| | | EGKKLVAASQAALGL |

hAIb-hIL2

| | | |
|---|---|---|
| 5 | amino | MKWVTFISLLFLFSSAYSRGVFRRDAHKSE |
| | acid | VAHRFKDLGEENFKALVLIAFAQYLQQCPF |
| | sequence | EDHVKLVNEVTEFAKTCVADESAENCDKSL |
| | | HTLFGDKLCTVATLRETYGEMADCCAKQEP |
| | | ERNECFLQHKDDNPNLPRLVRPEVDVMCTA |
| | | FHDNEETFLKKYLYEIARRHPYFYAPELLF |
| | | FAKRYKAAFTECCQAADKAACLLPKLDELR |
| | | DEGKASSAKQRLKCASLQKFGERAFKAWAV |
| | | ARLSQRFPKAEFAEVSKLVTDLTKVHTECC |
| | | HGDLLECADDRADLAKYICENQDSISSKLK |
| | | ECCEKPLLEKSHCIAEVENDEMPADLPSLA |
| | | ADFVESKDVCKNYAEAKDVFLGMFLYEYAR |
| | | RHPDYSVVLLLRLAKTYETTLEKCCAAADP |
| | | HECYAKVFDEFKPLVEEPQNLIKQNCELFE |
| | | QLGEYKFQNALLVRYTKKVPQVSTPTLVEV |
| | | SRNLGKVGSKCCKHPEAKRMPCAEDYLSVV |
| | | LNQLCVLHEKTPVSDRVTKCCTESLVNRRP |
| | | CFSALEVDETYVPKEFNAETFTFHADICTL |
| | | SEKERQIKKQTALVELVKHKPKATKEQLKA |
| | | VMDDFAAFVEKCCKADDKETCFAEEGKKLV |
| | | AASQAALGLGGSGGGGSGGAPTSSSTKKTQ |
| | | LQLEHLLLDLQMILNGINNYKNPKLTRMLT |
| | | FKFYMPKKATELKHLQCLEEELKPLEEVLN |
| | | LAQSKNFHLRPRDLISNINVIVLELKGSET |
| | | TFMCEYADETATIVEFLNRWITFCQSIIST |
| | | LT |

hAIb-hIL2_A4s8

| | | |
|---|---|---|
| 6 | amino | MKWVTFISLLFLFSSAYSRGVFRRDAHKSE |
| | acid | VAHRFKDLGEENFKALVLIAFAQYLQQCPF |
| | sequence | EDHVKLVNEVTEFAKTCVADESAENCDKSL |
| | | HTLFGDKLCTVATLRETYGEMADCCAKQEP |
| | | ERNECFLQHKDDNPNLPRLVRPEVDVMCTA |
| | | FHDNEETFLKKYLYEIARRHPYFYAPELLF |
| | | FAKRYKAAFTECCQAADKAACLLPKLDELR |
| | | DEGKASSAKQRLKCASLQKFGERAFKAWAV |
| | | ARLSQRFPKAEFAEVSKLVTDLTKVHTECC |
| | | HGDLLECADDRADLAKYICENQDSISSKLK |
| | | ECCEKPLLEKSHCIAEVENDEMPADLPSLA |
| | | ADFVESKDVCKNYAEAKDVFLGMFLYEYAR |
| | | RHPDYSVVLLLRLAKTYETTLEKCCAAADP |
| | | HECYAKVFDEFKPLVEEPQNLIKQNCELFE |
| | | QLGEYKFQNALLVRYTKKVPQVSTPTLVEV |
| | | SRNLGKVGSKCCKHPEAKRMPCAEDYLSVV |
| | | LNQLCVLHEKTPVSDRVTKCCTESLVNRRP |
| | | CFSALEVDETYVPKEFNAETFTFHADICTL |
| | | SEKERQIKKQTALVELVKHKPKATKEQLKA |
| | | VMDDFAAFVEKCCKADDKETCFAEEGKKLV |
| | | AASQAALGLGGSGGGGSGGAPTSSSTKKTQ |
| | | LQLEHLLLDLQMILNGINNYKNPKLTRMLT |
| | | FEFYMPKKATELKHLQCLEKELKPLEEVLN |
| | | LAHSKNFHFEPRDVISNINVFVLELKGSET |
| | | TFMCEYADETATIVEFLNRWITFCQSIIST |
| | | LT |

hIL7 hAIb

| | | |
|---|---|---|
| 7 | amino | MFHVSFRYIFGLPPLILVLLPVASSDCDIE |
| | acid | GKDGKQYESVLMVSIDQLLDSMKEIGSNCL |
| | sequence | NNEFNFFKRHICDANKEGMFLFRAARKLRQ |
| | | FLKMNSTGDFDLHLLKVSEGTTILLNCTGQ |
| | | VKGRKPAALGEAQPTKSLEENKSLKEQKKL |
| | | NDLCFLKRLLQEIKTCWNKILMGTKEHGGS |
| | | GGGGSGGDAHKSEVAHRFKDLGEENFKALV |
| | | LIAFAQYLQQCPFEDHVKLVNEVTEFAKTC |
| | | VADESAENCDKSLHTLFGDKLCTVATLRET |
| | | YGEMADCCAKQEPERNECFLQHKDDNPNLP |
| | | RLVRPEVDVMCTAFHDNEETFLKKYLYEIA |
| | | RRHPYFYAPELLFFAKRYKAAFTECCQAAD |
| | | KAACLLPKLDELRDEGKASSAKQRLKCASL |
| | | QKFGERAFKAWAVARLSQRFPKAEFAEVSK |
| | | LVTDLTKVHTECCHGDLLECADDRADLAKY |
| | | ICENQDSISSKLKECCEKPLLEKSHCIAEV |
| | | ENDEMPADLPSLAADFVESKDVCKNYAEAK |
| | | DVFLGMFLYEYARRHPDYSVVLLLRLAKTY |
| | | ETTLEKCCAAADPHECYAKVFDEFKPLVEE |
| | | PQNLIKQNCELFEQLGEYKFQNALLVRYTK |
| | | KVPQVSTPTLVEVSRNLGKVGSKCCKHPEA |
| | | KRMPCAEDYLSVVLNQLCVLHEKTPVSDRV |
| | | TKCCTESLVNRRPCFSALEVDETYVPKEFN |
| | | AETFTFHADICTLSEKERQIKKQTALVELV |
| | | KHKPKATKEQLKAVMDDFAAFVEKCCKADD |
| | | KETCFAEEGKKLVAASQAALGL |

Sec/MITD

| | | |
|---|---|---|
| 8 | Sec | MRVMAPRTLILLLSGALALTETWAGS |
| | (amino | |
| | acid) | |
| 9 | MITD (amino | IVGIVAGLAVLAVVVIGAVVATVMCRRKSS |
| | acid) | GGKGGSYSQAASSDSAQGSDVSLTA |

P2P16

| | | |
|---|---|---|
| 10 | P2P16 (amino | KKQYIKANSKFIGITELKKLGGGKRGGGKK |
| | acid) | MTNSVDDALINSTKIYSYFPSVISKVNQGA |
| | | QGKKL |

GS Linker

| | | |
|---|---|---|
| 11 | GS Linker 1 | GGSGGGGSGG |
| 12 | GS Linker 2 | GSSGGGGSPGGGSS |

5′-UTR (hAg-Kozak)

| | | |
|---|---|---|
| 13 | 5′-UTR | AACUAGUAUUCUUCUGGUCCCCACAGACUC |
| | | AGAGAGAACCCGCCACC |

3′-UTR (FI element)

| | | |
|---|---|---|
| 14 | 3′-UTR | CUGGUACUGCAUGCACGCAAUGCUAGCUGC |
| | | CCCUUUCCCGUCCUGGGUACCCCGAGUCUC |
| | | CCCCGACCUCGGGUCCCAGGUAUGCUCCCA |
| | | CCUCCACCUGCCCCACUCACCACCUCUGCU |
| | | AGUUCCAGACACCUCCCAAGCACGCAGCAA |
| | | UGCAGCUCAAAACGCUUAGCCUAGCCACAC |
| | | CCCCACGGGAAACAGCAGUGAUUAACCUUU |
| | | AGCAAUAAACGAAAGUUUAACUAAGCUAUA |
| | | CUAACCCCAGGGUUGGUCAAUUUCGUGCCA |
| | | GCCACACC |

A30L70

| | | |
|---|---|---|
| 15 | A30L70 | AAAAAAAAAAAAAAAAAAAAAAAAAAAAAA |
| | | GCAUAUGACUAAAAAAAAAAAAAAAAAAAA |
| | | AAAAAAAAAAAAAAAAAAAAAAAAAAAAAA |
| | | AAAAAAAAAAAAAAAAAAAA |

Helper epitopes

| | | |
|---|---|---|
| 16 | P2 | QYIKANSKFIGITEL |
| 17 | P16 | MTNSVDDALINSTKIYSYFPSVISKVNQGA |
| | | QG |

hAIb-hIL2

| | | |
|---|---|---|
| 18 | RNA | agacgaacuaguauucuucugguccccaca |
| | | gacucagagagaacccgccaccaugaagug |
| | | gguaaccuuuauuucccuucuuuuucucuu |
| | | uagcucggcuuauuccagggguguguuucg |
| | | ucgagaugcacacaagagugagguugcuca |
| | | ucgcuuuaaagauuugggagaagaaaauuu |
| | | caaagccuugguguugauugccuuugcuca |
| | | guaucuucagcaguguccauuugaagauca |
| | | uguaaaauuagugaaugaaguaacugaauu |
| | | ugcaaaaacauguguugcugaugagucagc |
| | | ugaaaauugugacaaaucacuucauacccu |
| | | uuuuggagacaaauuaugcacaguugcaac |
| | | acuucgugaaaccuauggugaaauggcuga |
| | | cugcugugcaaaacaagaaccugagagaaa |
| | | ugaaugcuucuugcaacacaaagaugacaa |
| | | cccaaaccucccccgauuggugagaccaga |
| | | gguugaugugaugugcacugcuuuucauga |
| | | caaugaagaaacauuuuugaaaaaauacuu |
| | | auaugaaauugccagaagacauccuuacuu |
| | | uuaugccccggaacuccuuuucuuugcuaa |
| | | aagguauaaagcugcuuuuacagaauguug |
| | | ccaagcugcugauaaagcugccugccuguu |
| | | gccaaagcucgaugaacuucgggaugaagg |
| | | gaaggcuucgucugccaaacagagacucaa |
| | | gugugccagucuccaaaaauuuggagaaag |
| | | agcuuucaaagcaugggcaguagcucgccu |
| | | gagccagagauuucccaaagcugaguuugc |
| | | agaaguuuccaaguuagugacagaucuuac |
| | | caaaguccacacggaaugcugccauggaga |
| | | ucugcuugaaugugcugaugacagggcgga |
| | | ccuugccaaguauaucugugaaaaucaaga |
| | | uucgaucuccaguaaacugaaggaaugcug |
| | | ugaaaaaccacuguuggaaaaaucccacug |
| | | cauugccgaaguggaaaaugaugagaugcc |
| | | ugcugacuugccuucauuagcugcugauuu |
| | | uguugaaaguaaggauguuugcaaaaacua |
| | | ugcugaggcaaaggaugucuuccugggcau |
| | | guuuuuguaugaauaugcaagaaggcaucc |
| | | ugauuacucugucgugcugcugcugagacu |
| | | ugccaagacauaugaaaccacucuagagaa |
| | | gugcugugccgcugcagauccucaugaaug |
| | | cuaugccaaaguguucgaugaauuuaaacc |
| | | ucuuguggaggagccucagaauuuaaucaa |
| | | acaaaauugugccuaggaaaagugggcagc |
| | | aaauguuguaaacauccugaagcaaaaaga |
| | | augcccugugcagaagacuaucuauccgug |
| | | guccugaaccaguuauguguguugcaugag |
| | | agcuuuuugagcagcuuggagaguacaaau |
| | | uccagaaugcgcuauuaguucguuacacca |
| | | agaaaguaccccaagugucaacuccaacuc |
| | | uuguagaggucucaagaaaaaaacgccagu |
| | | aagugacagagucaccaaaugcugcacaga |
| | | auccuuggugaacaggcgaccaugcuuuuc |
| | | agcucuggaagucgaugaaacauacguucc |
| | | caaagaguuuaaugcugaaacauucaccuu |
| | | ccaugcagauauaugcacacuuucugagaa |
| | | ggagagacaaaucaagaaacaaacugcacu |
| | | uguugagcuggugaaacacaagcccaaggc |
| | | aacaaaagagcaacugaaagcuguuaugga |
| | | ugauuucgcagcuuuuguagagaagugcug |
| | | caaggcugacgauaaggagaccugcuuugc |
| | | cgaggaggguaaaaaacuuguugcugcaag |
| | | ucaagcugccuuaggcuuaggcggcucugg |
| | | aggaggcggcuccggaggcgcuccaacauc |
| | | uucuucaacaaagaaaacacagcuucagcu |
| | | ugaacaccuucuucuugaucuucagaugau |
| | | ucugaauggaaucaacaauuacaaaaaucc |
| | | aaaacugacaagaaugcugacauuuaaauu |
| | | uuacaugccaaagaaagcaacagaacugaa |
| | | acaccuucagugccuugaagaagaacugaa |
| | | accucuggaagaagugcugaaucuggcuca |
| | | gagcaaaaauuuucaccugagaccaagaga |
| | | ucugaucagcaacaucaaugugauugugcu |
| | | ggaacugaaaggaucugaaacaacauucau |
| | | gugugaauaugcugaugaaacagcaacaau |
| | | uguggaauuucugaacagauggauuacauu |
| | | uugccagucaaucauuucaacacugacaug |
| | | augacucgagcugguacugcaugcacgcaa |
| | | ugcuagcugccccuuucccguccuggguac |
| | | cccgagucucccccgaccucgggucccagg |
| | | uaugcucccaccuccaccugccccacucac |
| | | caccucugcuaguuccagacaccucccaag |
| | | cacgcagcaaugcagcucaaaacgcuuagc |
| | | cuagccacacccccacgggaaacagcagug |
| | | auuaaccuuuagcaauaaacgaaaguuuaa |
| | | cuaagcuauacuaaccccaggguuggucaa |
| | | uuucgugccagccacaccgagaccuggucc |
| | | agagucgcuagccgcgucgcuaaaaaaaaa |
| | | aaaaaaaaaaaaaaaaaaaaagcauaugac |
| | | uaaaaaaaaaaaaaaaaaaaaaaaaaaaaa |
| | | aaaaaaaaaaaaaaaaaaaaaaaaaaaaaa |
| | | aaaaaaaaaaa |

hAIb-hIL2_A4s8

| | | |
|---|---|---|
| 19 | RNA | AGACGAACUAGUAUUCUUCUGGUCCCCACA |
| | | GACUCAGAGAGAACCCGCCACCAUGAAGUG |
| | | GGUAACCUUUAUUUCCCUUCUUUUUCUCUU |
| | | UAGCUCGGCUUAUUCCAGGGGUGUGUUUCG |
| | | UCGAGAUGCACACAAGAGUGAGGUUGCUCA |
| | | UCGCUUUAAAGAUUUGGGAGAAGAAAAUUU |
| | | CAAAGCCUUGGUGUUGAUUGCCUUUGCUCA |
| | | GUAUCUUCAGCAGUGUCCAUUUGAAGAUCA |
| | | UGUAAAAUUAGUGAAUGAAGUAACUGAAUU |
| | | UGCAAAAACAUGUGUUGCUGAUGAGUCAGC |
| | | UGAAAAUUGUGACAAAUCACUUCAUACCCU |
| | | UUUUGGAGACAAAUUAUGCACAGUUGCAAC |
| | | ACUUCGUGAAACCUAUGGUGAAAUGGCUGA |
| | | CUGCUGUGCAAAACAAGAACCUGAGAGAAA |
| | | UGAAUGCUUCUUGCAACACAAAGAUGACAA |
| | | CCCAAACCUCCCCCGAUUGGUGAGACCAGA |
| | | GGUUGAUGUGAUGUGCACUGCUUUUCAUGA |
| | | CAAUGAAGAAACAUUUUUGAAAAAAUACUU |
| | | AUAUGAAAUUGCCAGAAGACAUCCUUACUU |
| | | UUAUGCCCCGGAACUCCUUUUCUUUGCUAA |
| | | AAGGUAUAAAGCUGCUUUUACAGAAUGUUG |
| | | CCAAGCUGCUGAUAAAGCUGCCUGCCUGUU |
| | | GCCAAAGCUCGAUGAACUUCGGGAUGAAGG |
| | | GAAGGCUUCGUCUGCCAAACAGAGACUCAA |
| | | GUGUGCCAGUCUCCAAAAAUUUGGAGAAAG |
| | | AGCUUUCAAAGCAUGGGCAGUAGCUCGCCU |
| | | GAGCCAGAGAUUUCCCAAAGCUGAGUUUGC |
| | | AGAAGUUUCCAAGUUAGUGACAGAUCUUAC |
| | | CAAAGUCCACACGGAAUGCUGCCAUGGAGA |
| | | UCUGCUUGAAUGUGCUGAUGACAGGGCGGA |
| | | CCUUGCCAAGUAUAUCUGUGAAAAUCAAGA |
| | | UUCGAUCUCCAGUAAACUGAAGGAAUGCUG |
| | | UGAAAAACCACUGUUGGAAAAAUCCCACUG |
| | | CAUUGCCGAAGUGGAAAAUGAUGAGAUGCC |
| | | UGCUGACUUGCCUUCAUUAGCUGCUGAUUU |
| | | UGUUGAAAGUAAGGAUGUUUGCAAAAACUA |
| | | UGCUGAGGCAAAGGAUGUCUUCCUGGGCAU |
| | | GUUUUUGUAUGAAUAUGCAAGAAGGCAUCC |
| | | UGAUUACUCUGUCGUGCUGCUGCUGAGACU |
| | | UGCCAAGACAUAUGAAACCACUCUAGAGAA |
| | | GUGCUGUGCCGCUGCAGAUCCUCAUGAAUG |
| | | CUAUGCCAAAGUGUUCGAUGAAUUUAAACC |
| | | UCUUGUGGAGGAGCCUCAGAAUUUAAUCAA |
| | | ACAAAAUUGUGAGCUUUUUGAGCAGCUUGG |
| | | AGAGUACAAAUUCCAGAAUGCGCUAUUAGU |
| | | UCGUUACACCAAGAAAGUACCCCAAGUGUC |
| | | AACUCCAACUCUUGUAGAGGUCUCAAGAAA |
| | | CCUAGGAAAAGUGGGCAGCAAAUGUUGUAA |
| | | ACAUCCUGAAGCAAAAAGAAUGCCCUGUGC |
| | | AGAAGACUAUCUAUCCGUGGUCCUGAACCA |
| | | GUUAUGUGUGUUGCAUGAGAAAACGCCAGU |
| | | AAGUGACAGAGUCACCAAAUGCUGCACAGA |
| | | AUCCUUGGUGAACAGGCGACCAUGCUUUUC |
| | | AGCUCUGGAAGUCGAUGAAACAUACGUUCC |
| | | CAAAGAGUUUAAUGCUGAAACAUUCACCUU |
| | | CCAUGCAGAUAUAUGCACACUUUCUGAGAA |
| | | GGAGAGACAAAUCAAGAAACAAACUGCACU |
| | | UGUUGAGCUGGUGAAACACAAGCCCAAGGC |
| | | AACAAAAGAGCAACUGAAAGCUGUUAUGGA |
| | | UGAUUUCGCAGCUUUUGUAGAGAAGUGCUG |
| | | CAAGGCUGACGAUAAGGAGACCUGCUUUGC |
| | | CGAGGAGGGUAAAAAACUUGUUGCUGCAAG |
| | | UCAAGCUGCCUUAGGCUUAGGCGGCUCUGG |
| | | AGGAGGCGGCUCCGGAGGCGCUCCAACAUC |
| | | UUCUUCAACAAAGAAAACACAGCUUCAGCU |
| | | UGAACACCUUCUUCUUGAUCUUCAGAUGAU |
| | | UCUGAAUGGAAUCAACAAUUACAAAAAUCC |
| | | AAAACUGACAAGAAUGCUGACAUUUGAAUU |
| | | UUACAUGCCAAAGAAAGCAACAGAACUGAA |
| | | ACACCUUCAGUGCCUUGAAAAAGAACUGAA |
| | | ACCUCUGGAAGAAGUGCUGAAUCUGGCUCA |
| | | CAGCAAAAAUUUUCACUUUGAACCAAGAGA |
| | | UGUGAUCAGCAACAUCAAUGUGUUUGUGCU |
| | | GGAACUGAAAGGAUCUGAAACAACAUUCAU |
| | | GUGUGAAUAUGCUGAUGAAACAGCAACAAU |
| | | UGUGGAAUUUCUGAACAGAUGGAUUACAUU |
| | | UUGCCAGUCAAUCAUUUCAACACUGACAUG |
| | | AUGACUCGAGCUGGUACUGCAUGCACGCAA |
| | | UGCUAGCUGCCCCUUUCCCGUCCUGGGUAC |
| | | CCCGAGUCUCCCCCGACCUCGGGUCCCAGG |
| | | UAUGCUCCCACCUCCACCUGCCCCACUCAC |
| | | CACCUCUGCUAGUUCCAGACACCUCCCAAG |
| | | CACGCAGCAAUGCAGCUCAAAACGCUUAGC |
| | | CUAGCCACACCCCCACGGGAAACAGCAGUG |
| | | AUUAACCUUUAGCAAUAAACGAAAGUUUAA |
| | | CUAAGCUAUACUAACCCCAGGGUUGGUCAA |
| | | UUUCGUGCCAGCCACACCGAGACCUGGUCC |
| | | AGAGUCGCUAGCCGCGUCGCUAAAAAAAAA |
| | | AAAAAAAAAAAAAAAAAAAAAGCAUAUGAC |
| | | UAAAAAAAAAAAAAAAAAAAAAAAAAAAAA |
| | | AAAAAAAAAAAAAAAAAAAAAAAAAAAAAA |
| | | AAAAAAAAAAA |

hIL7 hAIb

| | | |
|---|---|---|
| 20 | RNA | agacgaacuaguauucuucugguccccaca |
| | | gacucagagagaacccgccaccauguucca |
| | | uguuuuuuuagguauaucuuuggacuuccu |
| | | ccccugauccuuguucuguugccaguagca |
| | | ucaucugauugugauauugaagguaaagau |
| | | ggcaaacaauaugagaguguucuaaugguc |
| | | agcaucgaucaauuauuggacagcaugaaa |
| | | gaaauugguagcaauugccugaauaaugaa |
| | | uuuaacuuuuuuaaaagacauaucugugau |
| | | gcuaauaaggaagguauguuuuuauuccgu |
| | | gcugcucgcaaguugaggcaauuucuuaaa |
| | | augaauagcacuggugauuuugaucuccac |
| | | uuauuaaaaguuucagaaggcacaacaaua |
| | | cuguugaacugcacuggccagguuaaagga |
| | | agaaaaccagcugcccugggugaagcccaa |
| | | ccaacaaagaguuuggaagaaaauaaaucu |
| | | uuaaaggaacagaaaaaacugaaugacuug |
| | | uguuuccuaaagagacuauuacaagagaua |
| | | aaaacuuguuggaauaaaauuuugaugggc |
| | | acuaaagaacacggcggcucuggaggaggc |
| | | ggcuccggaggcgaugcacacaagagugag |
| | | guugcucaucgcuuuaaagauuugggagaa |
| | | gaaaauuucaaagccuugguguugauugcc |
| | | uuugcucaguaucuucagcaguguccauuu |
| | | gaagaucauguaaaauuagugaaugaagua |
| | | acugaauuugcaaaaacauguguugcugau |
| | | gagucagcugaaaauugugacaaaucacuu |
| | | cauacccuuuuuggagacaaauuaugcaca |
| | | guugcaacacuucgugaaaccuauggugaa |
| | | auggcugacugcugugcaaaacaagaaccu |
| | | gagagaaaugaaugcuucuugcaacacaaa |
| | | gaugacaacccaaaccucccccgauuggug |
| | | agaccagagguugaugugaugugcacugcu |
| | | uuucaugacaaugaagaaacauuuuugaaa |
| | | aaauacuuauaugaaauugccagaagacau |
| | | ccuuacuuuuaugccccggaacuccuuuuc |
| | | uuugcuaaaagguauaaagcugcuuuuaca |
| | | gaauguugccaagcugcugauaaagcugcc |
| | | ugccuguugccaaagcucgaugaacuucgg |
| | | gaugaagggaaggcuucgucugccaaacag |
| | | agacucaagugugccagucuccaaaaauuu |
| | | ggagaaagagcuuucaaagcaugggcagua |
| | | gcucgccugagccagagauuucccaaagcu |
| | | gaguuugugaaaaucaagauucgaucucca |
| | | guaaacugaaggaaugcugugaaaaaccac |
| | | uguuggaaaaaucccacugcauugccgaag |
| | | uggaaaaugaugagaugccugcugaccaga |
| | | aguuuccaaguuagugacagaucuuaccaa |
| | | aguccacacggaaugcugccauggagaucu |
| | | gcuugaaugugcugaugacagggggaccuu |
| | | gccaaguauaucuguugccuucauuagcug |
| | | cugauuuuguugaaaguaaggauguuugca |
| | | aaaacuaugcugaggcaaaggaugucuucc |
| | | ugggcauguuuuuguaugaauaugcaagaa |
| | | ggcauccugauuacucugucgugcugcugc |
| | | ugagacuugccaagacauaugaaaccacuc |
| | | uagagaagugcugugccgcugcagauccuc |
| | | augaaugcuaugccaaaguguucgaugaau |
| | | uuaaaccucuuguggaggagccucagaauu |
| | | uaaucaaacaaaauugugagcuuuuugagc |
| | | agcuuggagaguacaaauuccagaaugcgc |
| | | uauuaguucguuacaccaagaaaguacccc |
| | | aagugucaacuccaacucuuguagaggucu |
| | | caagaaaccuaggaaaagugggcagcaaau |
| | | guuguaaacauccugaagcaaaaagaaugc |
| | | ccugugcagaagacuaucuauccguggucc |
| | | ugaaccaguuauguguguugcaugagaaaa |
| | | cgccaguaagugacagagucaccaaaugcu |
| | | gcacagaauccuuggugaacaggcgaccau |
| | | gcuuuucagcucuggaagucgaugaaacau |
| | | acguucccaaagaguuuaaugcugaaacau |
| | | ucaccuuccaugcagauauaugcacacuuu |
| | | cugagaaggagagacaaaucaagaaacaaa |
| | | cugcacuuguugagcuggugaaacacaagc |
| | | ccaaggcaacaaaagagcaacugaaagcug |
| | | uuauggaugauuucgcagcuuuuguagaga |
| | | agugcugcaaggcugacgauaaggagaccu |
| | | gcuuugccgaggaggguaaaaaacuuguug |
| | | cugcaagucaagcugccuuaggcuuaugau |
| | | gacucgagcugguacugcaugcacgcaaug |
| | | cuagcugccccuuucccguccuggguaccc |
| | | cgagucucccccgaccucgggucccaggua |
| | | ugcucccaccuccaccugccccacucacca |
| | | ccucugcuaguuccagacaccucccaagca |
| | | cgcagcaaugcagcucaaaacgcuuagccu |
| | | agccacacccccacgggaaacagcagugau |
| | | uaaccuuuagcaauaaacgaaaguuuaacu |
| | | aagcuauacuaaccccaggguuggucaauu |
| | | ucgugccagccacaccgagaccugguccag |
| | | agucgcuagccgcgucgcuaaaaaaaaaaa |
| | | aaaaaaaaaaaaaaaaaaagcauaugacua |
| | | aaaaaaaaaaaaaaaaaaaaaaaaaaaaaa |
| | | aaaaaaaaaaaaaaaaaaaaaaaaaaaaaa |
| | | aaaaaaaaa |

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means ±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of” or “consisting essentially of”. Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

In the following, definitions will be provided which apply to all aspects of the present disclosure. The following terms have the following meanings unless otherwise indicated. Any undefined terms have their art recognized meanings.

Terms such as “reduce”, “decrease”, “inhibit” or “impair” as used herein relate to an overall reduction or the ability to cause an overall reduction, preferably of at least 5%, at least 10%, at least 20%, at least 50%, at least 75% or even more, in the level. These terms include a complete or essentially complete inhibition, i.e., a reduction to zero or essentially to zero.

Terms such as “increase”, “enhance” or “exceed” preferably relate to an increase or enhancement by at least 10%, at least 20%, at least 30%, at least 40%, at least 50%, at least 80%, at least 100%, at least 200%, at least 500%, or even more.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 150 amino acids, but the terms “peptide”, “protein” and “polypeptide” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, immunostimulants and epitopes for vaccination.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

According to the invention, the term “modification” with respect to an amino acid sequence relates to a sequence change in an amino acid sequence compared to a parental sequence such as the sequence of a wildtype peptide, polypeptide or protein resulting in a variant of the parental sequence.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and phenylalanine, tyrosine.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, -2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence. Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to immunostimulants, one particular function is one or more immunostimulatory activities displayed by the amino acid sequence from which the fragment or variant is derived. With respect to antigens or antigenic sequences, one particular function is one or more immunogenic activities (e.g., specificity of the immune reaction) displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., stimulating or inducing an immune response. In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., immunostimulatory activity or immunogenicity of the functional variant may be at least 50%, at least 60%, at least 70%, at least 80%, or at least 90% of the parent molecule or sequence. However, in other embodiments, function of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the amino acid sequences suitable for use herein may be altered such that they vary in sequence from the naturally occurring or native sequences from which they were derived, while retaining the desirable activity of the native sequences.

As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present invention means “made through genetic engineering”. Preferably, a “recombinant object” such as a recombinant nucleic acid in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

“Physiological pH” as used herein refers to a pH of about 7.5.

The term “genetic modification” or simply “modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding immunostimulant or antigen is transiently transfected into cells. RNA can be transfected into cells to transiently express its coded protein.

As used herein the term “3D-P-DMA” preferably refers to (6Z,16Z)-12-((Z)-dec-4-en-1-yl)docosa-6,16-dien-11-yl 5-(dimethylamino)pentanoate.

As used herein, the term “PEG₂₀₀₀-C-DMA” preferably refers to 3-N-[(ω-Methoxy poly(ethylene glycol)2000) carbamoyl]-1,2-dimyristyloxy-propylamine (MPEG-(2 kDa)-C-DMA or Methoxy-polyethylene glycol-2,3-bis(tetradecyloxy)propylcarbamate (2000)).

As used herein, the term “DSPC” preferably refers to 1,2-Distearoyl-sn-glycero-3-phosphocholine (1,2-dioctadecanoyl-sn-glycero-3-phosphocholine, or PC (18:0/18:0)).

As used herein the term “Cholesterol” refers to Cholest-5-en-3β-ol (3S-Hydroxy-5-cholestene5-Cholesten-3β-ol).

### Immunostimulants

The present invention comprises the use of RNA encoding an amino acid sequence comprising a cytokine protein (immunostimulant RNA). The cytokine protein may be a naturally occurring cytokine or a functional variant thereof or a functional fragment of the cytokine or functional variant. In one embodiment, the cytokine protein induces an unwanted response or reaction, wherein the unwanted response or reaction may involve NK cells and may comprise one or more selected from the group consisting of increase in NK cell number, fever, malaise, reduction of body weight, increase in activity of liver enzymes, capillary leak syndrome, hypotension and edema. In one embodiment, the liver enzymes comprise one or more selected from the group consisting of alanine-aminotransferase (ALAT), aspartate-aminotransferase (ASAT), and lactate-dehydrogenase (LDH). In one embodiment, the cytokine protein induces NK cell expansion. In one embodiment, the cytokine protein is selected from the group consisting of IL2, IL15 and IL18, a functional variant thereof, or a functional fragment thereof.

In one embodiment, the amino acid sequence comprising a cytokine protein comprises an amino acid sequence comprising IL2, a functional variant thereof, or a functional fragment of the IL2 or the functional variant. In one embodiment, the present invention comprises the use of RNA encoding an amino acid sequence comprising human IL2, a functional variant thereof, or a functional fragment of the human IL2 or the functional variant thereof.

The immunostimulant RNA administered herein may further comprise RNA encoding an amino acid sequence comprising IL7, a functional variant thereof, or a functional fragment of the IL7 or the functional variant thereof. In one embodiment, the immunostimulant RNA administered herein may further comprise RNA encoding an amino acid sequence comprising human IL7, a functional variant thereof, or a functional fragment of the human IL7 or the functional variant thereof.

The methods and agents described herein are particularly effective if the immunostimulant portion (e.g., IL2 or IL7) is attached to a pharmacokinetic modifying group (hereafter referred to as “extended-pharmacokinetic (PK)” immunostimulant). In one embodiment, said RNA is targeted to the liver for systemic availability. Liver cells can be efficiently transfected and are able to produce large amounts of protein.

An “immunostimulant” is any substance that stimulates the immune system by inducing activation or increasing activity of any of the immune system's components, in particular immune effector cells.

Cytokines are a category of small proteins (˜5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumour necrosis factors but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. Cytokines act through receptors, and are especially important in the immune system; cytokines modulate the balance between humoral and cell-based immune responses, and they regulate the maturation, growth, and responsiveness of particular cell populations. Some cytokines enhance or inhibit the action of other cytokines in complex ways.

Interleukins (ILs) are a group of cytokines (secreted proteins and signal molecules) that can be divided into four major groups based on distinguishing structural features. However, their amino acid sequence similarity is rather weak (typically 15-25% identity). The human genome encodes more than 50 interleukins and related proteins.

Interleukin-2 (IL2) is a cytokine that induces proliferation of antigen-activated T cells and stimulates natural killer (NK) cells. The biological activity of IL2 is mediated through a multi-subunit IL2 receptor complex (IL2R) of three polypeptide subunits that span the cell membrane: p55 (IL2Rα, the alpha subunit, also known as CD25 in humans), p75 (IL2Rβ, the beta subunit, also known as CD122 in humans) and p64 (IL2Rγ, the gamma subunit, also known as CD 132 in humans). T cell response to IL2 depends on a variety of factors, including: (1) the concentration of IL2; (2) the number of IL2R molecules on the cell surface; and (3) the number of IL2R occupied by IL2 (i.e., the affinity of the binding interaction between IL2 and IL2R (Smith, “Cell Growth Signal Transduction is Quantal” In Receptor Activation by Antigens, Cytokines, Hormones, and Growth Factors 766:263-271, 1995)). The IL2:IL2R complex is internalized upon ligand binding and the different components undergo differential sorting. When administered as an intravenous (i.v.) bolus, IL2 has a rapid systemic clearance (an initial clearance phase with a half-life of 12.9 minutes followed by a slower clearance phase with a half-life of 85 minutes) (Konrad et al., Cancer Res. 50:2009-2017, 1990).

Outcomes of systemic IL2 administration in cancer patients are far from ideal. While 15 to 20 percent of patients respond objectively to high-dose IL2, the great majority do not, and many suffer severe, life-threatening side effects, including nausea, confusion, hypotension, and septic shock. Attempts to reduce serum concentration by reducing dose and adjusting dosing regimen have been attempted, and while less toxic, such treatments were also less efficacious. According to the disclosure, an IL2 may be a naturally occurring IL2 such as human IL2 (hIL2). In one embodiment, human IL2 comprises the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1.

In one embodiment, IL2 or an IL2 fragment or variant binds to the IL2 receptor.

According to the disclosure, in certain embodiments, IL2, a functional variant thereof, or a functional fragment of the IL2 or the functional variant is attached to a pharmacokinetic modifying group. The resulting molecule, hereafter referred to as “extended-pharmacokinetic (PK) IL2,” has a prolonged circulation half-life relative to free IL2, a functional variant thereof, or a functional fragment of the IL2 or the functional variant. The prolonged circulation half-life of extended-PK IL2 permits in vivo serum IL2 concentrations to be maintained within a therapeutic range, potentially leading to the enhanced activation of many types of immune cells, including T cells. Because of its favorable pharmacokinetic profile, extended-PK IL2 can be dosed less frequently and for longer periods of time when compared with unmodified IL2. In certain embodiments, the pharmacokinetic modifying group of the extended-PK IL2 is human albumin (hAlb), in particular if the IL2 is human IL2.

In one embodiment, hAlb comprises the amino acid sequence of SEQ ID NO: 3 or 4, an amino acid sequence having at least 99%, 98%, 97%, 96%, 95%, 90%, 85%, or 80% identity to the amino acid sequence of SEQ ID NO: 3 or 4, or a functional fragment of the amino acid sequence of SEQ ID NO: 3 or 4, or the amino acid sequence having at least 99%, 98%, 97%, 96%, 95%, 90%, 85%, or 80% identity to the amino acid sequence of SEQ ID NO: 3 or 4.

IL7 is a hematopoietic growth factor secreted by stromal cells in the bone marrow and thymus. It is also produced by keratinocytes, dendritic cells, hepatocytes, neurons, and epithelial cells, but is not produced by normal lymphocytes. IL7 is a cytokine important for B and T cell development. IL7 cytokine and the hepatocyte growth factor form a heterodimer that functions as a pre-pro-B cell growth-stimulating factor. Knockout studies in mice suggested that IL7 plays an essential role in lymphoid cell survival.

IL7 binds to the IL7 receptor, a heterodimer consisting of IL7 receptor a and common γ chain receptor. Binding results in a cascade of signals important for T cell development within the thymus and survival within the periphery. Knockout mice which genetically lack IL7 receptor exhibit thymic atrophy, arrest of T cell development at the double positive stage, and severe lymphopenia. Administration of IL7 to mice results in an increase in recent thymic emigrants, increases in B and T cells, and increased recovery of T cells after cyclophosphamide administration or after bone marrow transplantation.

According to the disclosure, an IL7 may be a naturally occurring IL7 such as human IL7 (hIL7). In one embodiment, human IL7 comprises the amino acid sequence of SEQ ID NO: 2, an amino acid sequence having at least 99%, 98%, 97%, 96%, 95%, 90%, 85%, or 80% identity to the amino acid sequence of SEQ ID NO: 2, or a functional fragment of the amino acid sequence of SEQ ID NO: 2, or the amino acid sequence having at least 99%, 98%, 97%, 96%, 95%, 90%, 85%, or 80% identity to the amino acid sequence of SEQ ID NO: 2.

In one embodiment, IL7 or an IL7 fragment or variant binds to the IL7 receptor.

According to the disclosure, in certain embodiments, IL7, a functional variant thereof, or a functional fragment of the IL7 or the functional variant is attached to a pharmacokinetic modifying group. The resulting molecule, hereafter referred to as “extended-pharmacokinetic (PK) IL7,” has a prolonged circulation half-life relative to free IL7, a functional variant thereof, or a functional fragment of the IL7 or the functional variant. The prolonged circulation half-life of extended-PK IL7 permits in vivo serum IL7 concentrations to be maintained within a therapeutic range, potentially leading to the enhanced activation of many types of immune cells, including T cells. Because of its favorable pharmacokinetic profile, extended-PK IL7 can be dosed less frequently and for longer periods of time when compared with unmodified IL7. In certain embodiments, the pharmacokinetic modifying group of the extended-PK IL7 is human albumin (hAlb), in particular if the IL7 is human IL7.

In one embodiment, hAlb comprises the amino acid sequence of SEQ ID NO: 3 or 4, an amino acid sequence having at least 99%, 98%, 97%, 96%, 95%, 90%, 85%, or 80% identity to the amino acid sequence of SEQ ID NO: 3 or 4, or a functional fragment of the amino acid sequence of SEQ ID NO: 3 or 4, or the amino acid sequence having at least 99%, 98%, 97%, 96%, 95%, 90%, 85%, or 80% identity to the amino acid sequence of SEQ ID NO: 3 or 4.

The immunostimulant RNA, i.e., RNA encoding an amino acid sequence comprising a cytokine protein, e.g., RNA encoding an amino acid sequence comprising IL2, a functional variant thereof, or a functional fragment of the IL2 or the functional variant, described herein encodes polypeptide comprising an immunostimulant portion, e.g., a cytokine or functional variant, or functional fragment thereof. The immunostimulant portion may be an IL2-derived immunostimulant portion or IL2 immunostimulant portion or an IL7-derived immunostimulant portion or IL7 immunostimulant portion. The IL2 immunostimulant portion may be IL2, a functional variant thereof, or a functional fragment of the IL2 or the functional variant thereof. The IL7 immunostimulant portion may be IL7, a functional variant thereof, or a functional fragment of the IL7 or the functional variant thereof.

Thus, the polypeptide comprising an immunostimulant portion may be an IL2 immunostimulant polypeptide (also designated herein “amino acid sequence comprising IL2, a functional variant thereof, or a functional fragment of the IL2 or the functional variant thereof”) or an IL7 immunostimulant polypeptide (also designated herein “amino acid sequence comprising IL7, a functional variant thereof, or a functional fragment of the IL7 or the functional variant thereof”).

In one embodiment, an IL2 immunostimulant polypeptide comprises the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1. In one embodiment, an IL2 immunostimulant polypeptide comprises the amino acid sequence of SEQ ID NO: 1.

In one embodiment, an IL2 immunostimulant polypeptide comprises a variant of a naturally occurring IL2 such as human IL2. In particular, the variant of a naturally occurring IL2 comprises a mutein of human IL2 or of a functional variant of human IL2. In one embodiment, the human IL2 or functional variant thereof is substituted such that affinity for the βγ IL2 receptor complex (IL2Rβγ) is enhanced. In one embodiment, the human IL2 or functional variant thereof is further substituted such that affinity for the αβγ IL2 receptor complex (IL2Rαβγ) is reduced. In one embodiment, variants of IL2 activate effector T cells over regulatory T cells. Specifically, variants of IL2 may contain mutations enhancing IL2Rβγ binding, in particular CD122 binding (“mutβγ”) and optionally further contain mutations affecting IL2Rαβγ binding, in particular CD25 binding (“mutα”). In particular, variants of IL2 may show reduced expansion of Treg cells and increased effector T cell and NK-cell stimulation, preferably IL2Rβγ+ effector T cell and NK-cell stimulation. The IL2 variant hAlb-hIL2_A4s8 described herein shows, compared to wild-type IL2, a markedly reduced capacity to activate Treg cells and increased ability to stimulate effector immune cells, preferably IL2Rβγ+ effector immune cells like CD8+ T cells and NK cells already at lower concentrations.

In one embodiment, an IL2 immunostimulant polypeptide comprises a mutein of human IL2 or of a functional variant of human IL2, wherein the human IL2 or the functional variant thereof is substituted at at least position 80 (leucine), position 81 (arginine), position 85 (leucine) and position 92 (isoleucine) relative to wild type human IL2 and numbered in accordance with wild type human IL2, wherein the substitution enhances the affinity for the βγ IL2 receptor complex (IL2Rβγ). In one embodiment, the human IL2 or the functional variant thereof is not substituted at position 86 (isoleucine) relative to wild type human IL2 and numbered in accordance with wild type human IL2.

In one embodiment, position 80 (leucine) is substituted by phenylalanine, position 81 (arginine) is substituted by glutamic acid, position 85 (leucine) is substituted by valine and position 92 (isoleucine) is substituted by phenylalanine relative to wild type human IL2 and numbered in accordance with wild type human IL2.

In one embodiment, the human IL2 or the functional variant thereof is further substituted at position 74 (glutamine) relative to wild type human IL2 and numbered in accordance with wild type human IL2. In one embodiment, position 74 (glutamine) is substituted by histidine relative to wild type human IL2 and numbered in accordance with wild type human IL2.

In one embodiment, an IL2 immunostimulant polypeptide comprises a mutein of human IL2 or of a functional variant of human IL2, wherein the human IL2 or the functional variant thereof is substituted at at least position 80 (leucine) by phenylalanine, position 81 (arginine) by glutamic acid, position 85 (leucine) by valine and position 92 (isoleucine) by phenylalanine relative to wild type human IL2 and numbered in accordance with wild type human IL2.

In one embodiment, the human IL2 or the functional variant thereof is further substituted at position 74 (glutamine) by histidine relative to wild type human IL2 and numbered in accordance with wild type human IL2.

In one embodiment, an IL2 immunostimulant polypeptide comprises a mutein of human IL2 or of a functional variant of human IL2, wherein the human IL2 or the functional variant thereof is substituted at at least position 74 (glutamine) by histidine, position 80 (leucine) by phenylalanine, position 81 (arginine) by glutamic acid, position 85 (leucine) by valine and position 92 (isoleucine) by phenylalanine relative to wild type human IL2 and numbered in accordance with wild type human IL2.

In one embodiment, the substitution enhances the affinity for IL2Rβγ.

In one embodiment, the substituted IL2 or functional variant thereof (IL2 mutein) described above has an amino acid sequence identical to wild type IL2 at the other, non-substituted residues. In one embodiment, the IL2 mutein described above has amino acid modifications such as amino acid substitutions at one or more sites in or at the other residues of wild type human IL2. In one embodiment, such amino acid substitutions result in relatively decreased affinity for IL2Rαβγ when compared to wild type IL2 (also termed “mutα” mutations herein). In one embodiment, such amino acid substitutions are at amino acid residues that contact IL2Rα.

Thus, in one embodiment, the human IL2 or the functional variant thereof further comprises one or more amino acid substitutions which reduce the affinity for the alpha subunit of the αβγ IL2 receptor complex (IL2Rαβγ).

In one embodiment, the one or more amino acid substitutions which reduce the affinity for the alpha subunit of IL2Rαβγ reduce the affinity for IL2Rαβγ to a greater extent than for IL2Rβγ.

In one embodiment, the one or more amino acid substitutions which reduce the affinity for the alpha subunit of IL2Rαβγ comprise substitutions of the human IL2 or the functional variant thereof at at least one of positions 35 (lysine), 43 (lysine), 61 (glutamic acid) and 62 (glutamic acid) relative to wild type human IL2 and numbered in accordance with wild type human IL2.

In one embodiment, if the amino acid residue is an acidic amino acid residue in wild type human IL2 the substitution is by a basic amino acid residue and if the amino acid residue is a basic amino acid residue in wild type human IL2 the substitution is by an acidic amino acid residue.

In different embodiments, the one or more amino acid substitutions which reduce the affinity for the alpha subunit of IL2Rαβγ comprise substitutions of the human IL2 or the functional variant thereof at at least the following positions relative to wild type human IL2 and numbered in accordance with wild type human IL2:
position 35,
position 43,
position 61,
position 62,
position 35 and position 43,
position 35 and position 61,
position 35 and position 62,
position 43 and position 61,
position 43 and position 62,
position 61 and position 62,
position 35, position 43 and position 61,
position 35, position 43 and position 62,
position 35, position 61 and position 62,
position 43, position 61 and position 62, or
position 35, position 43, position 61 and position 62.

In one embodiment, position 35 is substituted with glutamic acid. In one embodiment, position 43 is substituted with glutamic acid. In one embodiment, position 61 is substituted with lysine. In one embodiment, position 62 is substituted with lysine.

In one embodiment, position 35 is substituted. In one embodiment, position 35 is substituted with glutamic acid.

In one embodiment, position 43 is substituted. In one embodiment, position 43 is substituted with glutamic acid.

In one embodiment, position 61 is substituted. In one embodiment, position 61 is substituted with lysine.

In one embodiment, position 62 is substituted. In one embodiment, position 62 is substituted with lysine.

In one embodiment, positions 43 and 61 are substituted. In one embodiment, position 43 is substituted with glutamic acid and position 61 is substituted with lysine.

In one embodiment, positions 35, 43 and 61 are substituted. In one embodiment, position 35 is substituted with glutamic acid, position 43 is substituted with glutamic acid and position 61 is substituted with lysine.

In one embodiment, positions 61 and 62 are substituted. In one embodiment, position 61 is substituted with lysine and position 62 is substituted with lysine.

In one embodiment, the one or more amino acid substitutions which reduce the affinity for the alpha subunit of IL2Rαβγ comprise substitutions of the human IL2 or the functional variant thereof at position 43 (lysine) and position 61 (glutamic acid) relative to wild type human IL2 and numbered in accordance with wild type human IL2. In one embodiment, position 43 (lysine) is substituted by glutamic acid and position 61 (glutamic acid) is substituted by lysine.

In one embodiment, the present disclosure provides for the administration of RNA encoding an amino acid sequence comprising a mutein of human IL2 or of a functional variant of human IL2, wherein the human IL2 or the functional variant thereof is substituted at at least position 43 (lysine) by glutamic acid, position 61 (glutamic acid) by lysine, position 74 (glutamine) by histidine, position 80 (leucine) by phenylalanine, position 81 (arginine) by glutamic acid, position 85 (leucine) by valine and position 92 (isoleucine) by phenylalanine relative to wild type human IL2 and numbered in accordance with wild type human IL2.

In one embodiment, the human IL2 or the functional variant thereof is not substituted at position 86 (isoleucine) relative to wild type human IL2 and numbered in accordance with wild type human IL2.

In one embodiment, the wild type human IL2 has the amino acid sequence according to SEQ ID NO: 1.

In one embodiment, the mutein of human IL2 or the functional variant thereof has a decreased ability to stimulate regulatory T cells compared to wild type human IL2.

In one embodiment, the mutein of human IL2 or the functional variant thereof has an increased ability to stimulate effector T cells compared to wild type human IL2.

The IL2 mutein described herein may be attached to a pharmacokinetic modifying group and, thus, may be an “extended-pharmacokinetic (PK) IL2”.

In one embodiment, the polypeptide described herein is an extended pharmacokinetic (PK) polypeptide. In one embodiment, the extended-PK polypeptide comprises a fusion protein. In one embodiment, the fusion protein comprises a moiety of the mutein of human IL2 or the functional variant thereof and a moiety which is heterologous to human IL2 or the functional variant thereof. In one embodiment, the fusion protein comprises a moiety of the mutein of human IL2 or the functional variant thereof and a moiety selected from the group consisting of serum albumin, an immunoglobulin fragment, transferrin, Fn3, and variants thereof. In one embodiment, the serum albumin comprises mouse serum albumin or human serum albumin. In one embodiment, the immunoglobulin fragment comprises an immunoglobulin Fc domain. As used herein, “IL2 mutein” means a variant of IL2 (including functional variants thereof), in particular a polypeptide wherein specific substitutions to the IL2 protein have been made.

In one embodiment, substitutions to the human IL2 protein have been made enhancing IL2Rβγ (“mutβγ”) binding, in particular CD122 binding. For example, the IL2 muteins may be characterized by amino acid substitutions of the native IL2 polypeptide chain such amino acid substitutions resulting, for example, in relatively increased affinity for IL2Rβγ when compared to wild type IL2, such that IL2 mediated stimulation no longer requires engagement of the IL2Rα. Such mutants are potent IL2 signaling agonists. Particularly preferred embodiments include the following: leucine (Leu) residue at position 80, arginine (Arg) residue at position 81, leucine (Leu) residue at position 85 and isoleucine (Ile) residue at position 92, relative to wild type human IL2 and numbered in accordance with wild type human IL2.

In one embodiment, further substitutions to the human IL2 protein have been made affecting IL2Rαβγ binding, in particular CD25 binding (“mutα”). For example, the IL2 muteins may also be characterized by amino acid substitutions of the native IL2 polypeptide chain such amino acid substitutions resulting, for example, in relatively decreased affinity for IL2Rαβγ, in particular the a subunit thereof, when compared to wild type IL2 (i.e., the IL2 muteins in addition to the “mutβδγ” mutations also comprise “mutα” mutations). These mutations can be at amino acid residues that contact IL2Rα. Particularly preferred embodiments include the following: lysine (Lys) residue at position 35, lysine (Lys) residue at position 43, glutamic acid (Glu) residue at position 61 and glutamic acid (Glu) residue at position 62, relative to wild type human IL2 and numbered in accordance with wild type human IL2, or any combination thereof.

IL2 muteins may have an amino acid sequence identical to wild type IL2 at the other, non-substituted residues (i.e., the IL2 muteins comprise “mutβγ” and optionally “mutα” mutations). However, the IL2 muteins may also be characterized by amino acid insertions, deletions, substitutions and modifications at one or more sites in or at the other residues of the native IL2 polypeptide chain. In accordance with this invention any such insertions, deletions, substitutions and modifications may result in an IL2 mutein that has enhanced affinity for IL2Rβγ while optionally having reduced affinity for IL2Rαβγ.

The substituted amino acid residue(s) can be, but are not necessarily, conservative substitutions.

By “numbered in accordance with wild type IL2” we mean identifying a chosen amino acid with reference to the position at which that amino acid normally occurs in the mature sequence of wild type IL2. Where insertions or deletions are made to the IL2 mutein, one of skill in the art will appreciate that an amino acid normally occurring at a certain position may be shifted in position in the mutein. However, the location of the shifted amino acid can be readily determined by inspection and correlation of the flanking amino acids with those flanking the amino acid in wild type IL2.

In one embodiment, an IL2 immunostimulant polypeptide comprises the amino acid sequence of amino acids 620 to 752 of SEQ ID NO: 6, an amino acid sequence having at least 99%, 98%, 97%, 96%, 95%, 90%, 85%, or 80% identity to the amino acid sequence of amino acids 620 to 752 of SEQ ID NO: 6, or a functional fragment of the amino acid sequence of amino acids 620 to 752 of SEQ ID NO: 6, or the amino acid sequence having at least 99%, 98%, 97%, 96%, 95%, 90%, 85%, or 80% identity to the amino acid sequence of amino acids 620 to 752 of SEQ ID NO: 6. In one embodiment, an IL2 immunostimulant polypeptide comprises the amino acid sequence of amino acids 620 to 752 of SEQ ID NO: 6.

In one embodiment, an IL7 immunostimulant polypeptide comprises the amino acid sequence of SEQ ID NO: 2, an amino acid sequence having at least 99%, 98%, 97%, 96%, 95%, 90%, 85%, or 80% identity to the amino acid sequence of SEQ ID NO: 2, or a functional fragment of the amino acid sequence of SEQ ID NO: 2, or the amino acid sequence having at least 99%, 98%, 97%, 96%, 95%, 90%, 85%, or 80% identity to the amino acid sequence of SEQ ID NO: 2. In one embodiment, an IL7 immunostimulant polypeptide comprises the amino acid sequence of SEQ ID NO: 2.

In one embodiment, an IL7 immunostimulant polypeptide comprises the amino acid sequence of amino acids 1 to 177 of SEQ ID NO: 7, an amino acid sequence having at least 99%, 98%, 97%, 96%, 95%, 90%, 85%, or 80% identity to the amino acid sequence of amino acids 1 to 177 of SEQ ID NO: 7, or a functional fragment of the amino acid sequence of amino acids 1 to 177 of SEQ ID NO: 7, or the amino acid sequence having at least 99%, 98%, 97%, 96%, 95%, 90%, 85%, or 80% identity to the amino acid sequence of amino acids 1 to 177 of SEQ ID NO: 7. In one embodiment, an IL7 immunostimulant polypeptide comprises the amino acid sequence of amino acids 1 to 177 of SEQ ID NO: 7.

In one embodiment, albumin such as human albumin (hAlb) is fused, either directly or through a linker, to an immunostimulant portion.

In one embodiment, hAlb comprises the amino acid sequence of SEQ ID NO: 3 or 4, an amino acid sequence having at least 99%, 98%, 97%, 96%, 95%, 90%, 85%, or 80% identity to the amino acid sequence of SEQ ID NO: 3 or 4, or a functional fragment of the amino acid sequence of SEQ ID NO: 3 or 4, or the amino acid sequence having at least 99%, 98%, 97%, 96%, 95%, 90%, 85%, or 80% identity to the amino acid sequence of SEQ ID NO: 3 or 4. In one embodiment, hAlb comprises the amino acid sequence of SEQ ID NO: 3 or 4.

hAlb is preferably used in order to promote prolonged circulation half-life of the immunostimulant portion. Accordingly, in particularly preferred embodiments, the immunostimulant RNA described herein comprises at least one coding region encoding an immunostimulant portion and a coding region encoding hAlb, said hAlb preferably being fused to the immunostimulant portion, e.g., to the N-terminus and/or the C-terminus of the immunostimulant portion. In one embodiment, hAlb and the immunostimulant portion are separated by a linker such as a GS linker, e.g. a GS linker having the amino acid sequence of SEQ ID NO: 11.

In one embodiment, an IL2 immunostimulant polypeptide comprises the amino acid sequence of amino acids 25 to 752 of SEQ ID NO: 5 or 6, an amino acid sequence having at least 99%, 98%, 97%, 96%, 95%, 90%, 85%, or 80% identity to the amino acid sequence of amino acids 25 to 752 of SEQ ID NO: 5 or 6, or a functional fragment of the amino acid sequence of amino acids 25 to 752 of SEQ ID NO: 5 or 6, or the amino acid sequence having at least 99%, 98%, 97%, 96%, 95%, 90%, 85%, or 80% identity to the amino acid sequence of amino acids 25 to 752 of SEQ ID NO: 5 or 6. In one embodiment, an IL2 immunostimulant polypeptide comprises the amino acid sequence of amino acids 25 to 752 of SEQ ID NO: 5 or 6.

In one embodiment, an IL7 immunostimulant polypeptide comprises the amino acid sequence of amino acids 26 to 772 of SEQ ID NO: 7, an amino acid sequence having at least 99%, 98%, 97%, 96%, 95%, 90%, 85%, or 80% identity to the amino acid sequence of amino acids 26 to 772 of SEQ ID NO: 7, or a functional fragment of the amino acid sequence of amino acids 26 to 772 of SEQ ID NO: 7, or the amino acid sequence having at least 99%, 98%, 97%, 96%, 95%, 90%, 85%, or 80% identity to the amino acid sequence of amino acids 26 to 772 of SEQ ID NO: 7. In one embodiment, an IL7 immunostimulant polypeptide comprises the amino acid sequence of amino acids 26 to 772 of SEQ ID NO: 7.

According to certain embodiments, a signal peptide is fused, either directly or through a linker, to an immunostimulant portion which is optionally fused to an extended-PK group, e.g., albumin, in particular hAlb.

Such signal peptides are sequences, which typically exhibit a length of about 15 to 30 amino acids and are preferably located at the N-terminus of the polypeptide to which it is fused, without being limited thereto. Signal peptides as defined herein preferably allow the transport of the peptide or protein it is fused to into a defined cellular compartment, preferably the cell surface, the endoplasmic reticulum (ER) or the endosomal-lysosomal compartment.

In one embodiment, the signal peptide sequence as defined herein includes, without being limited thereto, the signal peptide sequence of an interleukin. In one embodiment, the signal peptide sequence as defined herein includes, without being limited thereto, the signal peptide sequence of the interleukin from which the immunostimulant portion is derived, in particular if the immunostimulant portion is the N-terminal portion of the immunostimulant polypeptide. Accordingly, the immunostimulant portion may be the non-mature IL, i.e., the IL containing its endogenous signal peptide.

In one embodiment, the signal peptide sequence as defined herein includes, without being limited thereto, the signal peptide sequence of an extended-PK group, e.g., albumin. In one embodiment, the signal peptide sequence as defined herein includes, without being limited thereto, the signal peptide sequence of the extended-PK group, e.g., albumin, from which the extended-PK group, e.g., albumin, is derived, in particular if the extended-PK group, e.g., albumin, is the N-terminal portion of the immunostimulant polypeptide. Accordingly, the extended-PK group, e.g., albumin, may be the non-mature extended-PK group, e.g., albumin, i.e., the extended-PK group, e.g., albumin, containing its endogenous signal peptide.

In one embodiment, a signal sequence comprises the amino acid sequence of amino acids 1 to 25 of SEQ ID NO: 7, an amino acid sequence having at least 99%, 98%, 97%, 96%, 95%, 90%, 85%, or 80% identity to the amino acid sequence of amino acids 1 to 25 of SEQ ID NO: 7, or a functional fragment of the amino acid sequence of amino acids 1 to 25 of SEQ ID NO: 7, or the amino acid sequence having at least 99%, 98%, 97%, 96%, 95%, 90%, 85%, or 80% identity to the amino acid sequence of amino acids 1 to 25 of SEQ ID NO: 7. In one embodiment, a signal sequence comprises the amino acid sequence of amino acids 1 to 25 of SEQ ID NO: 7.

In one embodiment, a signal sequence comprises the amino acid sequence of amino acids 1 to 18 of SEQ ID NO: 3, an amino acid sequence having at least 99%, 98%, 97%, 96%, 95%, 90%, 85%, or 80% identity to the amino acid sequence of amino acids 1 to 18 of SEQ ID NO: 3, or a functional fragment of the amino acid sequence of amino acids 1 to 18 of SEQ ID NO: 3, or the amino acid sequence having at least 99%, 98%, 97%, 96%, 95%, 90%, 85%, or 80% identity to the amino acid sequence of amino acids 1 to 18 of SEQ ID NO: 3. In one embodiment, a signal sequence comprises the amino acid sequence of amino acids 1 to 18 of SEQ ID NO: 3.

Such signal peptides are preferably used in order to promote secretion of the encoded polypeptide to which they are fused.

Accordingly, in particularly preferred embodiments, the RNA described herein comprises at least one coding region encoding an immunostimulant protein optionally fused to hAlb and a signal peptide, said signal peptide preferably being fused to immunostimulant protein optionally fused to hAlb, more preferably to the N-terminus of the immunostimulant protein optionally fused to hAlb.

In one embodiment an IL2 immunostimulant polypeptide comprises the amino acid sequence of SEQ ID NO: 5 or 6, an amino acid sequence having at least 99%, 98%, 97%, 96%, 95%, 90%, 85%, or 80% identity to the amino acid sequence of SEQ ID NO: 5 or 6, or a functional fragment of the amino acid sequence of SEQ ID NO: 5 or 6, or the amino acid sequence having at least 99%, 98%, 97%, 96%, 95%, 90%, 85%, or 80% identity to the amino acid sequence of SEQ ID NO: 5 or 6. In one embodiment, an IL2 immunostimulant polypeptide comprises the amino acid sequence of SEQ ID NO: 5 or 6.

In one embodiment, RNA encoding an IL2 immunostimulant polypeptide (i) comprises the nucleotide sequence of nucleotides 53 to 2308 of SEQ ID NO: 18, a nucleotide sequence having at least 99%, 98%, 97%, 96%, 95%, 90%, 85%, or 80% identity to the nucleotide sequence of nucleotides 53 to 2308 of SEQ ID NO: 18, or a fragment of the nucleotide sequence of nucleotides 53 to 2308 of SEQ ID NO: 18, or the nucleotide sequence having at least 99%, 98%, 97%, 96%, 95%, 90%, 85%, or 80% identity to the nucleotide sequence of nucleotides 53 to 2308 of SEQ ID NO: 18; and/or (ii) encodes an amino acid sequence comprising the amino acid sequence of SEQ ID NO: 5, an amino acid sequence having at least 99%, 98%, 97%, 96%, 95%, 90%, 85%, or 80% identity to the amino acid sequence of SEQ ID NO: 5, or a functional fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5. In one embodiment, RNA encoding an IL2 immunostimulant polypeptide (i) comprises the nucleotide sequence of nucleotides 53 to 2308 of SEQ ID NO: 18; and/or (ii) encodes an amino acid sequence comprising the amino acid sequence of SEQ ID NO: 5.

In one embodiment, RNA encoding an IL2 immunostimulant polypeptide (i) comprises the nucleotide sequence of SEQ ID NO: 18, a nucleotide sequence having at least 99%, 98%, 97%, 96%, 95%, 90%, 85%, or 80% identity to the nucleotide sequence of SEQ ID NO: 18, or a fragment of the nucleotide sequence of SEQ ID NO: 18, or the nucleotide sequence having at least 99%, 98%, 97%, 96%, 95%, 90%, 85%, or 80% identity to the nucleotide sequence of SEQ ID NO: 18; and/or (ii) encodes an amino acid sequence comprising the amino acid sequence of SEQ ID NO: 5, an amino acid sequence having at least 99%, 98%, 97%, 96%, 95%, 90%, 85%, or 80% identity to the amino acid sequence of SEQ ID NO: 5, or a functional fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5. In one embodiment, RNA encoding an IL2 immunostimulant polypeptide (i) comprises the nucleotide sequence of SEQ ID NO: 18; and/or (ii) encodes an amino acid sequence comprising the amino acid sequence of SEQ ID NO: 5.

In one embodiment, RNA encoding an IL2 immunostimulant polypeptide (i) comprises the nucleotide sequence of nucleotides 53 to 2308 of SEQ ID NO: 19, a nucleotide sequence having at least 99%, 98%, 97%, 96%, 95%, 90%, 85%, or 80% identity to the nucleotide sequence of nucleotides 53 to 2308 of SEQ ID NO: 19, or a fragment of the nucleotide sequence of nucleotides 53 to 2308 of SEQ ID NO: 19, or the nucleotide sequence having at least 99%, 98%, 97%, 96%, 95%, 90%, 85%, or 80% identity to the nucleotide sequence of nucleotides 53 to 2308 of SEQ ID NO: 19; and/or (ii) encodes an amino acid sequence comprising the amino acid sequence of SEQ ID NO: 6, an amino acid sequence having at least 99%, 98%, 97%, 96%, 95%, 90%, 85%, or 80% identity to the amino acid sequence of SEQ ID NO: 6, or a functional fragment of the amino acid sequence of SEQ ID NO: 6, or the amino acid sequence having at least 99%, 98%, 97%, 96%, 95%, 90%, 85%, or 80% identity to the amino acid sequence of SEQ ID NO: 6. In one embodiment, RNA encoding an IL2 immunostimulant polypeptide (i) comprises the nucleotide sequence of nucleotides 53 to 2308 of SEQ ID NO: 19; and/or (ii) encodes an amino acid sequence comprising the amino acid sequence of SEQ ID NO: 6.

In one embodiment, RNA encoding an IL2 immunostimulant polypeptide (i) comprises the nucleotide sequence of SEQ ID NO: 19, a nucleotide sequence having at least 99%, 98%, 97%, 96%, 95%, 90%, 85%, or 80% identity to the nucleotide sequence of SEQ ID NO: 19, or a fragment of the nucleotide sequence of SEQ ID NO: 19, or the nucleotide sequence having at least 99%, 98%, 97%, 96%, 95%, 90%, 85%, or 80% identity to the nucleotide sequence of SEQ ID NO: 19; and/or (ii) encodes an amino acid sequence comprising the amino acid sequence of SEQ ID NO: 6, an amino acid sequence having at least 99%, 98%, 97%, 96%, 95%, 90%, 85%, or 80% identity to the amino acid sequence of SEQ ID NO: 6, or a functional fragment of the amino acid sequence of SEQ ID NO: 6, or the amino acid sequence having at least 99%, 98%, 97%, 96%, 95%, 90%, 85%, or 80% identity to the amino acid sequence of SEQ ID NO: 6. In one embodiment, RNA encoding an IL2 immunostimulant polypeptide (i) comprises the nucleotide sequence of SEQ ID NO: 19; and/or (ii) encodes an amino acid sequence comprising the amino acid sequence of SEQ ID NO: 6.

In one embodiment, an IL7 immunostimulant polypeptide comprises the amino acid sequence of SEQ ID NO: 7, an amino acid sequence having at least 99%, 98%, 97%, 96%, 95%, 90%, 85%, or 80% identity to the amino acid sequence of SEQ ID NO: 7, or a functional fragment of the amino acid sequence of SEQ ID NO: 7, or the amino acid sequence having at least 99%, 98%, 97%, 96%, 95%, 90%, 85%, or 80% identity to the amino acid sequence of SEQ ID NO: 7. In one embodiment, an IL7 immunostimulant polypeptide comprises the amino acid sequence of SEQ ID NO: 7.

In one embodiment, RNA encoding an IL7 immunostimulant polypeptide (i) comprises the nucleotide sequence of nucleotides 53 to 2368 of SEQ ID NO: 20, a nucleotide sequence having at least 99%, 98%, 97%, 96%, 95%, 90%, 85%, or 80% identity to the nucleotide sequence of nucleotides 53 to 2368 of SEQ ID NO: 20, or a fragment of the nucleotide sequence of nucleotides 53 to 2368 of SEQ ID NO: 20, or the nucleotide sequence having at least 99%, 98%, 97%, 96%, 95%, 90%, 85%, or 80% identity to the nucleotide sequence of nucleotides 53 to 2368 of SEQ ID NO: 20; and/or (ii) encodes an amino acid sequence comprising the amino acid sequence of SEQ ID NO: 7, an amino acid sequence having at least 99%, 98%, 97%, 96%, 95%, 90%, 85%, or 80% identity to the amino acid sequence of SEQ ID NO: 7, or a functional fragment of the amino acid sequence of SEQ ID NO: 7, or the amino acid sequence having at least 99%, 98%, 97%, 96%, 95%, 90%, 85%, or 80% identity to the amino acid sequence of SEQ ID NO: 7. In one embodiment, RNA encoding an IL7 immunostimulant polypeptide (i) comprises the nucleotide sequence of nucleotides 53 to 2368 of SEQ ID NO: 20; and/or (ii) encodes an amino acid sequence comprising the amino acid sequence of SEQ ID NO: 7.

In one embodiment, RNA encoding an IL7 immunostimulant polypeptide (i) comprises the nucleotide sequence of SEQ ID NO: 20, a nucleotide sequence having at least 99%, 98%, 97%, 96%, 95%, 90%, 85%, or 80% identity to the nucleotide sequence of SEQ ID NO: 20, or a fragment of the nucleotide sequence of SEQ ID NO: 20, or the nucleotide sequence having at least 99%, 98%, 97%, 96%, 95%, 90%, 85%, or 80% identity to the nucleotide sequence of SEQ ID NO: 20; and/or (ii) encodes an amino acid sequence comprising the amino acid sequence of SEQ ID NO: 7, an amino acid sequence having at least 99%, 98%, 97%, 96%, 95%, 90%, 85%, or 80% identity to the amino acid sequence of SEQ ID NO: 7, or a functional fragment of the amino acid sequence of SEQ ID NO: 7, or the amino acid sequence having at least 99%, 98%, 97%, 96%, 95%, 90%, 85%, or 80% identity to the amino acid sequence of SEQ ID NO: 7. In one embodiment, RNA encoding an IL7 immunostimulant polypeptide (i) comprises the nucleotide sequence of SEQ ID NO: 20; and/or (ii) encodes an amino acid sequence comprising the amino acid sequence of SEQ ID NO: 7.

In the following, embodiments of the immunostimulant RNAs are described, wherein certain terms used when describing elements thereof have the following meanings:

hAg-Kozak: 5′-UTR sequence of the human alpha-globin mRNA with an optimized ‘Kozak sequence’ to increase translational efficiency.

SP: Signal peptide.

hAlb: Sequences encoding human albumin.

IL2/IL7: Sequences encoding the respective human IL or variant or fragment.

Linker (GS): Sequences coding for linker peptides predominantly consisting of the amino acids glycine (G) and serine (S), as commonly used for fusion proteins.

FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.

A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency.

In one embodiment, IL2 immunostimulant RNA described herein comprises the structure: hAgKozak-SP-hAlb-Linker-IL2 mature-FI element-Ligation3-A30LA70

In one embodiment, IL2 immunostimulant described herein comprises the structure: SP-hAlb-Linker-IL2 mature

In one embodiment, IL7 immunostimulant RNA described herein comprises the structure: hAgKozak-IL7 with SP-Linker-hAlb mature-FI element-Ligation3-A30LA70

In one embodiment, IL7 immunostimulant described herein comprises the structure: IL7 with SP-Linker-hAlb mature

In one embodiment, hAg-Kozak comprises the nucleotide sequence of SEQ ID NO: 13. In one embodiment, IL2 comprises the amino acid sequence of SEQ ID NO: 1. In one embodiment, IL2 comprises the amino acid sequence of amino acids 620 to 752 of SEQ ID NO: 6. In one embodiment, IL7 comprises the amino acid sequence of SEQ ID NO: 2. In one embodiment, hAlb comprises the amino acid sequence of SEQ ID NO: 3 or 4. In one embodiment, Linker comprises the amino acid sequence of SEQ ID NO: 11. In one embodiment, FI comprises the nucleotide sequence of SEQ ID NO: 14. In one embodiment, A30L70 comprises the nucleotide sequence of SEQ ID NO: 15. In one embodiment, the immunostimulant RNAs described herein contain 1-methyl-pseudouridine instead of uridine. The preferred 5′ cap structure is m₂^{7,3′-O}Gppp(m₁^{λ-O})ApG.

As discussed above, the immunostimulants described herein such as IL2 immunostimulant or IL7 immunostimulant are generally present as a fusion protein with an extended-PK group.

The term “fusion protein” as used herein refers to a polypeptide or protein comprising two or more subunits. Preferably, the fusion protein is a translational fusion between the two or more subunits. The translational fusion may be generated by genetically engineering the coding nucleotide sequence for one subunit in a reading frame with the coding nucleotide sequence of a further subunit. Subunits may be interspersed by a linker.

As used herein, the terms “linked,” “fused”, or “fusion” are used interchangeably. These terms refer to the joining together of two or more elements or components or domains.

Immunostimulant polypeptides described herein can be prepared as fusion or chimeric polypeptides that include an immunostimulant portion and a heterologous polypeptide (i.e., a polypeptide that is not an immunostimulant). The immunostimulant may be fused to an extended-PK group, which increases circulation half-life. Non-limiting examples of extended-PK groups are described infra. It should be understood that other PK groups that increase the circulation half-life of immunostimulants such as cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16(7):903-15 which is herein incorporated by reference in its entirety. As used herein, an “extended-PK” immunostimulant refers to an immunostimulant moiety in combination with an extended-PK group. In one embodiment, the extended-PK immunostimulant is a fusion protein in which an immunostimulant moiety is linked or fused to an extended-PK group.

In certain embodiments, the serum half-life of an extended-PK immunostimulant is increased relative to the immunostimulant alone (i.e., the immunostimulant not fused to an extended-PK group). In certain embodiments, the serum half-life of the extended-PK immunostimulant is at least 20, 40, 60, 80, 100, 120, 150, 180, 200, 400, 600, 800, or 1000% longer relative to the serum half-life of the immunostimulant alone. In certain embodiments, the serum half-life of the extended-PK immunostimulant is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the immunostimulant alone. In certain embodiments, the serum half-life of the extended-PK immunostimulant is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

As used herein, “half-life” refers to the time taken for the serum or plasma concentration of a compound such as a peptide or protein to reduce by 50%, in vivo, for example due to degradation and/or clearance or sequestration by natural mechanisms. An extended-PK immunostimulant suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular an immunostimulant. The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In one embodiment, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”. In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver, that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal.

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used.

In one embodiment, the therapeutic protein(s) is (are) joined to the albumin through (a) peptide linker(s). A linker peptide between the fused portions may provide greater physical separation between the moieties and thus maximize the accessibility of the therapeutic protein portion, for instance, for binding to its cognate receptor. The linker peptide may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species. Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

In certain aspects, the extended-PK immunostimulant, suitable for use according to the disclosure, can employ one or more peptide linkers. As used herein, the term “peptide linker” refers to a peptide or polypeptide sequence which connects two or more domains (e.g., the extended-PK moiety and an immunostimulant moiety) in a linear amino acid sequence of a polypeptide chain. For example, peptide linkers may be used to connect an immunostimulant moiety to a HSA domain.

Linkers suitable for fusing the extended-PK group to e.g. an immunostimulant are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

### Antigenic Epitopes

The present invention may comprises the use of RNA for vaccination, i.e., the use of RNA encoding one or more antigenic epitopes

In one embodiment, the one or more antigenic epitopes are derived from one or more tumor antigens. In one embodiment, the vaccine RNA comprises RNA of unimolecular species encoding one or more antigenic epitopes or RNA of different molecular species each RNA encoding at least one antigenic epitope. Thus, each RNA encodes an amino acid sequence comprising one or more epitopes. In one embodiment, the RNA molecules may encode a monoepitopic or polyepitopic polypeptide. If the polypeptide comprises more than one antigenic epitope said epitopes may be identical and/or different. Vaccine RNA may be translated in cells of the subject, in particular antigen presenting cells, to produce the encoded amino acid sequence. In one embodiment, following appropriate processing of the amino acid sequence by cells, the epitopes are presented by MHC and displayed to the subject's immune system for stimulation of appropriate T cells.

In one embodiment, the vaccine RNA, when administered to a patent, provides a collection of epitopes such as MHC presented epitopes, such as 2 or more, 5 or more, 10 or more, 15 or more, 20 or more, 25 or more, 30 or more and preferably up to 60, up to 55, up to 50, up to 45, up to 40, up to 35 or up to 30 epitopes, which epitopes may be derived from tumor antigens. Presentation of these epitopes by cells of a subject, in particular antigen presenting cells, may result in T cells targeting the epitopes when bound to MHC and may result in targeting the subject's tumor, preferably the primary tumor as well as tumor metastases, when expressing antigens from which the MHC presented epitopes are derived and presenting the same epitopes on the surface of the tumor cells.

For providing vaccine RNA, the invention may comprise the arbitrary inclusion of a sufficient number of epitopes which may be derived from one or more tumor antigens into the vaccine RNA (as coding sequence). The epitopes may be included as such or may be flanked by amino acid sequences also flanking said epitopes in the naturally occurring protein. In one embodiment, the epitopes are represented by the complete antigen (as coding sequence) from which they are derived. Such flanking sequences each may comprise 3 or more, 5 or more, 10 or more, 15 or more, 20 or more and preferably up to 50, up to 45, up to 40, up to 35 or up to 30 amino acids and may flank the epitope sequence N-terminally and/or C-terminally.

Vaccine RNA may encode epitopes in the form of a polyepitopic or multiepitopic polypeptide. In certain embodiments of the present disclosure, the polypeptide comprises at least two epitopes, at least three epitopes, at least four epitopes, at least five epitopes, at least six epitopes, at least seven epitopes, at least eight epitopes, at least nine epitopes, or at least ten epitopes being derived from the same and/or different tumor antigens. The epitopes may be present in the polypeptide in the form of a vaccine sequence, i.e. present in their natural sequence context, e.g. flanked by amino acid sequences also flanking the epitopes in the naturally occurring protein. Such flanking sequences each may comprise 3 or more, 5 or more, 10 or more, 15 or more, 20 or more and preferably up to 50, up to 45, up to 40, up to 35 or up to 30 amino acids and may flank the epitope sequence N-terminally and/or C-terminally. Thus, a vaccine sequence may comprise 20 or more, 25 or more, 30 or more, 35 or more, 40 or more and preferably up to 50, up to 45, up to 40, up to 35 or up to 30 amino acids. In one embodiment, the epitopes and/or vaccine sequences are lined up in the polypeptide head-to-tail.

In one embodiment, the epitopes and/or vaccine sequences are spaced by linkers, in particular neutral linkers. The term “linker” according to the invention relates to a peptide added between two peptide domains such as epitopes or vaccine sequences to connect said peptide domains. There is no particular limitation regarding the linker sequence. However, it is preferred that the linker sequence reduces steric hindrance between the two peptide domains, is well translated, and supports or allows processing of the epitopes. Furthermore, the linker should have no or only little immunogenic sequence elements. Linkers preferably should not create non-endogenous epitopes like those generated from the junction suture between adjacent epitopes, which might generate unwanted immune reactions. Therefore, the polyepitopic vaccine should preferably contain linker sequences which are able to reduce the number of unwanted MHC binding junction epitopes. Hoyt et al. (EMBO J. 25(8), 1720-9, 2006) and Zhang et al. (J. Biol. Chem., 279(10), 8635-41, 2004) have shown that glycine-rich sequences impair proteasomal processing and thus the use of glycine rich linker sequences act to minimize the number of linker-contained peptides that can be processed by the proteasome. Furthermore, glycine was observed to inhibit a strong binding in MHC binding groove positions (Abastado et al., J. Immunol. 151(7), 3569-75, 1993). Schlessinger et al. (Proteins, 61(1), 115-26, 2005) had found that the amino acids glycine and serine included in an amino acid sequence result in a more flexible protein that is more efficiently translated and processed by the proteasome, enabling better access to the encoded epitopes. The linker each may comprise 3 or more, 6 or more, 9 or more, 10 or more, 15 or more, 20 or more and preferably up to 50, up to 45, up to 40, up to 35 or up to 30 amino acids. Preferably the linker is enriched in glycine and/or serine amino acids. Preferably, at least 50%, at least 60%, at least 70%, at least 80%, at least 90%, or at least 95% of the amino acids of the linker are glycine and/or serine. In one preferred embodiment, a linker is substantially composed of the amino acids glycine and serine.

In one embodiment, the one or more antigenic epitopes provided by vaccine RNA are encoded by different RNA molecules encoding a collection of polypeptides comprising said epitopes on different polypeptides, wherein said polypeptides each comprise one or more epitopes, which can also be overlapping. In the case of vaccine RNA comprising different RNA molecules encoding more than one polyepitopic and/or multiepitopic polypeptide the epitopes provided by the different polypeptides may be different or partially overlapping.

Once present in cells of a subject such as antigen presenting cells the polypeptide according to the invention is processed to produce antigenic epitopes. Administration of vaccine RNA may provide MHC class II-presented epitopes that are capable of eliciting a CD4+ helper T cell response against cells expressing antigens from which the MHC presented epitopes are derived. Alternatively or additionally, administration of vaccine RNA may provide MHC class I-presented epitopes that are capable of eliciting a CD8+ T cell response against cells expressing antigens from which the MHC presented epitopes are derived. Preferably, vaccine RNA is useful for polyepitopic stimulation of cytotoxic and/or helper T cell responses.

RNA encoding one or more antigenic epitopes described herein used for vaccination preferably results in stimulation, priming and/or expansion of T cells in the subject being administered the RNA. Said stimulated, primed and/or expanded T cells are preferably directed against target antigen, in particular a target antigen expressed by cancer cells, tissues and/or organs, i.e., a tumor antigen.

According to the disclosure, a polypeptide comprising one or more antigenic epitopes may comprise a tumor antigen, or a fragment thereof (e.g., an epitope or vaccine sequence), or may comprise a variant of a tumor antigen or fragment thereof. In one embodiment, such variant is immunologically equivalent to the tumor antigen or fragment. In the context of the present disclosure, the term “variant of a tumor antigen or fragment thereof” means a sequence which results in stimulation, priming and/or expansion of T cells which stimulated, primed and/or expanded T cells target the tumor antigen, in particular when presented by diseased cells, tissues and/or organs. Thus, the polypeptide comprising one or more antigenic epitopes may correspond to or may comprise a tumor antigen, may correspond to or may comprise a fragment of a tumor antigen or may correspond to or may comprise an amino acid sequence which is homologous to a tumor antigen or a fragment thereof. If the polypeptide comprising one or more epitopes comprises a fragment of a tumor antigen or an amino acid sequence which is homologous to a fragment of a tumor antigen said fragment or amino acid sequence may comprise an epitope such as a T cell epitope of the tumor antigen or a sequence which is homologous to an epitope such as a T cell epitope of the tumor antigen. Thus, according to the disclosure, a polypeptide comprising one or more epitopes may comprise an immunogenic fragment of a tumor antigen or an amino acid sequence being homologous to an immunogenic fragment of a tumor antigen. An “immunogenic fragment of an antigen” according to the disclosure preferably relates to a fragment of an antigen which is capable of stimulating, priming and/or expanding T cells when presented in the context of MHC molecules. It is preferred that the polypeptide comprising one or more epitopes (similar to the tumor antigen) can be presented by a cell such as an antigen-presenting cell so as to provide the relevant epitope for binding by T cells.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject, such as T cells binding to the reference amino acid sequence or cells expressing the reference amino acid sequence, induces an immune reaction having a specificity of reacting with the reference amino acid sequence. Thus, a molecule which is immunologically equivalent to an antigen exhibits the same or essentially the same properties and/or exerts the same or essentially the same effects regarding stimulation, priming and/or expansion of T cells as the antigen to which the T cells are targeted.

In one embodiment, vaccine RNA described herein comprises one or more antigenic epitope-encoding RNAs. In one embodiment, vaccine RNA described herein comprises two or more antigenic epitope-encoding RNAs. In different embodiments, two or more includes 3 or more, 4 or more, 5 or more, 6 or more, 7 or more, 8 or more, 9 or more, or 10 or more. The two or more RNAs may be present in a mixture or may be present separate from each other and consequently may be administered separate from each other, e.g. at different time points and/or by different routes, to a subject.

The antigenic epitopes described herein may be derived from one or more tumor antigens. In one embodiment, the vaccine RNA described herein encodes one or more tumor antigens or variants or fragments thereof. In one embodiment, a tumor antigen does not differ in its amino acid sequence between cancer tissue and healthy tissue. Alternatively or additionally, a tumor antigen may be a neo-antigen, which is specific to an individual's tumor. In such embodiment, one or more antigenic epitopes may comprise neo-epitopes. A neo-antigen or neo-epitope may result from one or more cancer-specific mutations in the genome of cancer cells resulting in amino acid changes.

According to the invention, the term “neo-antigen” relates to a peptide or protein including one or more amino acid modifications compared to the parental peptide or protein. For example, the neo-antigen may be a tumor-associated neo-antigen, wherein the term “tumor-associated neo-antigen” includes a peptide or protein including amino acid modifications due to tumor-specific mutations, i.e., mutations in the nucleic acid of a cancer cell. Such mutations may be identified by known sequencing techniques.

According to the invention, the term “tumor-specific mutation” or “cancer-specific mutation” relates to a somatic mutation that is present in the nucleic acid of a tumor or cancer cell but absent in the nucleic acid of a corresponding normal, i.e. non-tumor or non-cancer, cell. The terms “tumor-specific mutation” and “tumor mutation” and the terms “cancer-specific mutation” and “cancer mutation” are used interchangeably herein.

In one embodiment, cancer-specific amino acid modifications are identified by identifying cancer-specific somatic mutations, e.g. by sequencing genomic DNA and/or RNA of cancer tissue or one or more cancer cells.

In one embodiment, the mutations are cancer-specific somatic mutations in a tumor specimen of a cancer patient which may be determined by identifying sequence differences between the genome, exome and/or transcriptome of a tumor specimen and the genome, exome and/or transcriptome of a non-tumorigenous specimen.

According to the invention a tumor specimen relates to any sample such as a bodily sample derived from a patient containing or being expected of containing tumor or cancer cells. The bodily sample may be any tissue sample such as blood, a tissue sample obtained from the primary tumor or from tumor metastases or any other sample containing tumor or cancer cells.

A non-tumorigenous specimen relates to any sample such as a bodily sample derived from a patient or another individual which preferably is of the same species as the patient, preferably a healthy individual not containing or not being expected of containing tumor or cancer cells. The bodily sample may be any tissue sample such as blood or a sample from a non-tumorigenous tissue.

Determining cancer-specific mutations may involve the determination of the cancer mutation signature of a patient. The term “cancer mutation signature” may refer to all cancer mutations present in one or more cancer cells of a patient or it may refer to only a portion of the cancer mutations present in one or more cancer cells of a patient. Accordingly, determining cancer-specific mutations may involve the identification of all cancer specific mutations present in one or more cancer cells of a patient or it may involve the identification of only a portion of the cancer specific mutations present in one or more cancer cells of a patient.

Preferably, the mutations identified are non-synonymous mutations, preferably non-synonymous mutations of proteins expressed in a tumor or cancer cell.

In one embodiment, the step of identifying cancer-specific somatic mutations or identifying sequence differences comprises single cell sequencing of one or more, preferably 2, 3, 4, 5, 6, 7, 8, 9, 10, 11, 12, 13, 14, 15, 16, 17, 18, 19, 20 or even more cancer cells. Thus, the invention may comprise identifying a cancer mutation signature of said one or more cancer cells. In one embodiment, the cancer cells are circulating tumor cells. The cancer cells such as the circulating tumor cells may be isolated prior to single cell sequencing.

In one embodiment, the step of identifying cancer-specific somatic mutations or identifying sequence differences involves using next generation sequencing (NGS).

In one embodiment, the step of identifying cancer-specific somatic mutations or identifying sequence differences comprises sequencing genomic DNA and/or RNA of the tumor specimen. To reveal cancer specific somatic mutations or sequence differences the sequence information obtained from the tumor specimen is preferably compared with a reference such as sequence information obtained from sequencing nucleic acid such as DNA or RNA of normal non-cancerous cells such as germline cells which may either be obtained from the patient or a different individual. In one embodiment, normal genomic germline DNA is obtained from peripheral blood mononuclear cells (PBMCs)

The term “genome” relates to the total amount of genetic information in the chromosomes of an organism or a cell.

The term “exome” refers to part of the genome of an organism formed by exons, which are coding portions of expressed genes. The exome provides the genetic blueprint used in the synthesis of proteins and other functional gene products. It is the most functionally relevant part of the genome and, therefore, it is most likely to contribute to the phenotype of an organism. The exome of the human genome is estimated to comprise 1.5% of the total genome (Ng, P C et al., PLoS Gen., 4(8): 1-15, 2008).

The term “transcriptome” relates to the set of all RNA molecules, including mRNA, rRNA, tRNA, and other non-coding RNA produced in one cell or a population of cells. In context of the present invention the transcriptome means the set of all RNA molecules produced in one cell, a population of cells, preferably a population of cancer cells, or all cells of a given individual at a certain time point.

The term “mutation” refers to a change of or difference in the nucleic acid sequence (nucleotide substitution, addition or deletion) compared to a reference. A “somatic mutation” can occur in any of the cells of the body except the germ cells (sperm and egg) and therefore are not passed on to children. These alterations can (but do not always) cause cancer or other diseases. Preferably, a mutation is a non-synonymous mutation. The term “non-synonymous mutation” refers to a mutation, preferably a nucleotide substitution, which does result in an amino acid change such as an amino acid substitution in the translation product.

As used herein, the term “vaccine” refers to a composition that induces an immune response upon inoculation into a subject. In some embodiments, the induced immune response provides therapeutic and/or protective immunity.

In one embodiment, vaccine RNA is expressed in cells of the subject to provide, optionally following procession of the expressed amino acid sequence, antigenic epitopes. In one embodiment, the antigenic epitopes are presented in the context of MHC. In one embodiment, the RNA encoding the antigenic epitopes is transiently expressed in cells of the subject. In one embodiment, after administration of the RNA encoding the antigenic epitopes, expression of the RNA encoding the antigenic epitopesin spleen occurs. In one embodiment, after administration of the RNA encoding the antigenic epitopes, expression of the RNA encoding the antigenic epitopesin antigen presenting cells, preferably professional antigen presenting cells occurs. In one embodiment, the antigen presenting cells are selected from the group consisting of dendritic cells, macrophages and B cells. In one embodiment, after administration of the RNA encoding the antigenic epitopes, no or essentially no expression of the RNA encoding the antigenic epitopes in lung and/or liver occurs. In one embodiment, after administration of the RNA encoding the antigenic epitopes, expression of the RNA encoding the antigenic epitopesin spleen is at least 5-fold the amount of expression in lung.

The antigenic epitopes described herein may be derived from a target antigen, i.e. an antigen against which an immune response is to be elicited. For example, antigenic epitopes may be fragments of a target antigen. The target antigen may be a tumor antigen.

Antigenic epitopes which may be provided to a subject according to the invention by administering vaccine RNA encoding antigenic epitopes or amino acid sequences comprising the antigenic epitopes preferably result in the induction of an immune response, e.g., a cellular immune response, and preferably results in stimulation, priming and/or expansion of T cells, in the subject being provided the antigenic epitopes. Said immune response is preferably directed against a target antigen when expressed by diseased cells, tissues and/or organs, i.e., a disease-associated antigen, in particular a tumor antigen.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which immune effector cells are stimulated by an antigen, proliferate, and the specific immune effector cell recognizing said antigen is amplified. Preferably, clonal expansion leads to differentiation of the immune effector cells.

The term “antigen” relates to an agent comprising an epitope against which an immune response can be generated. The term “antigen” includes, in particular, proteins and peptides. In one embodiment, an antigen is presented by cells of the immune system such as antigen presenting cells like dendritic cells or macrophages. An antigen or a procession product thereof such as a T cell epitope is in one embodiment bound by a T- or B-cell receptor, or by an immunoglobulin molecule such as an antibody. Accordingly, an antigen or a procession product thereof may react specifically with antibodies or T lymphocytes (T cells). In one embodiment, an antigen is a disease-associated antigen, such as a tumor antigen, and an epitope is derived from such antigen.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen or an epitope thereof may therefore be used for therapeutic purposes. Disease-associated antigens may be associated with cancer, typically tumors.

The antigen target may be upregulated during a disease, e.g. cancer. In diseased tissues, antigens can differ from healthy tissue and offer unique possibilities for early detection, specific diagnosis and therapy, especially targeted therapy.

In some embodiments the antigen is a tumor antigen.

In the context of the present invention, the term “tumor antigen” or “tumor-associated antigen” relates to proteins that are expressed or aberrantly expressed in one or more tumor or cancer tissues and preferably are under normal conditions specifically expressed in a limited number of tissues and/or organs or in specific developmental stages, for example, the tumor antigen may be under normal conditions specifically expressed in stomach tissue, preferably in the gastric mucosa, in reproductive organs, e.g., in testis, in trophoblastic tissue, e.g., in placenta, or in germ line cells. In this context, “a limited number” preferably means not more than 3, more preferably not more than 2. The tumor antigens in the context of the present invention include, for example, differentiation antigens, preferably cell type specific differentiation antigens, i.e., proteins that are under normal conditions specifically expressed in a certain cell type at a certain differentiation stage, cancer/testis antigens, i.e., proteins that are under normal conditions specifically expressed in testis and sometimes in placenta, and germ line specific antigens. In the context of the present invention, the tumor antigen is preferably associated with the cell surface of a cancer cell and is preferably not or only rarely expressed in normal tissues. Preferably, the tumor antigen or the aberrant expression of the tumor antigen identifies cancer cells. In the context of the present invention, the tumor antigen that is expressed by a cancer cell in a subject, e.g., a patient suffering from a cancer disease, is preferably a self-protein in said subject. In preferred embodiments, the tumor antigen in the context of the present invention is expressed under normal conditions specifically in a tissue or organ that is non-essential, i.e., tissues or organs which when damaged by the immune system do not lead to death of the subject, or in organs or structures of the body which are not or only hardly accessible by the immune system. Preferably, the amino acid sequence of the tumor antigen is identical between the tumor antigen which is expressed in normal tissues and the tumor antigen which is expressed in cancer tissues.

Examples for tumor antigens include p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY-BR-1, p190 minor BCR-abL, Pm1/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT. Particularly preferred tumor antigens include CLAUDIN-18.2 (CLDN18.2) and CLAUDIN-6 (CLDN6).

The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes and preferably relates to T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

As used herein the term “neo-epitope” refers to an epitope that is not present in a reference such as a normal non-cancerous or germline cell but is found in cancer cells. This includes, in particular, situations wherein in a normal non-cancerous or germline cell a corresponding epitope is found, however, due to one or more mutations in a cancer cell the sequence of the epitope is changed so as to result in the neo-epitope.

According to certain embodiments, a signal peptide is fused, either directly or through a linker, e.g., a linker having the amino acid sequence according to SEQ ID NO: 11, to an antigenic sequence encoded by the vaccine RNA described herein (including multi-epitope polypeptides as described above).

Such signal peptides are sequences, which typically exhibit a length of about 15 to 30 amino acids and are preferably located at the N-terminus of the antigenic sequence, without being limited thereto. Signal peptides as defined herein preferably allow the transport of the antigenic sequence as encoded by the RNA into a defined cellular compartment, preferably the cell surface, the endoplasmic reticulum (ER) or the endosomal-lysosomal compartment. In one embodiment, the signal peptide sequence as defined herein includes, without being limited thereto, the signal peptide sequence derived from the sequence encoding the human MHC class I complex (HLA-B51, haplotype A2, B27/B51, Cw2/Cw3), and preferably corresponds to the 78 bp fragment coding for the secretory signal peptide, which guides translocation of the nascent polypeptide chain into the endoplasmatic reticulum, and includes, in particular a sequence comprising the amino acid sequence of SEQ ID NO: 8 or a functional variant thereof.

In one embodiment, a signal sequence comprises the amino acid sequence of SEQ ID NO: 8, an amino acid sequence having at least 99%, 98%, 97%, 96%, 95%, 90%, 85%, or 80% identity to the amino acid sequence of SEQ ID NO: 8, or a functional fragment of the amino acid sequence of SEQ ID NO: 8, or the amino acid sequence having at least 99%, 98%, 97%, 96%, 95%, 90%, 85%, or 80% identity to the amino acid sequence of SEQ ID NO: 8. In one embodiment, a signal sequence comprises the amino acid sequence of SEQ ID NO: 8.

Such signal peptides are preferably used in order to promote secretion of the encoded antigenic sequence. More preferably, a signal peptide as defined herein is fused to an encoded antigenic sequence as defined herein.

Accordingly, in particularly preferred embodiments, the RNA described herein comprises at least one coding region encoding an antigenic sequence and a signal peptide, said signal peptide preferably being fused to the antigenic sequence, more preferably to the N-terminus of the antigenic sequence as described herein.

According to certain embodiments, an amino acid sequence enhancing antigen processing and/or presentation is fused, either directly or through a linker, to an antigenic sequence.

Such amino acid sequences enhancing antigen processing and/or presentation are preferably located at the C-terminus of the antigenic sequence (and optionally at the C-terminus of an amino acid sequence which breaks immunological tolerance), without being limited thereto. Amino acid sequences enhancing antigen processing and/or presentation as defined herein preferably improve antigen processing and presentation. In one embodiment, the amino acid sequence enhancing antigen processing and/or presentation as defined herein includes, without being limited thereto, sequences derived from the human MHC class I complex (HLA-B51, haplotype A2, B27/B51, Cw2/Cw3), in particular a sequence comprising the amino acid sequence of SEQ ID NO: 9 or a functional variant thereof.

In one embodiment, an amino acid sequence enhancing antigen processing and/or presentation comprises the amino acid sequence of SEQ ID NO: 9, an amino acid sequence having at least 99%, 98%, 97%, 96%, 95%, 90%, 85%, or 80% identity to the amino acid sequence of SEQ ID NO: 9, or a functional fragment of the amino acid sequence of SEQ ID NO: 9, or the amino acid sequence having at least 99%, 98%, 97%, 96%, 95%, 90%, 85%, or 80% identity to the amino acid sequence of SEQ ID NO: 9. In one embodiment, an amino acid sequence enhancing antigen processing and/or presentation comprises the amino acid sequence of SEQ ID NO: 9.

Such amino acid sequences enhancing antigen processing and/or presentation are preferably used in order to promote antigen processing and/or presentation of the encoded antigenic sequence. More preferably, an amino acid sequence enhancing antigen processing and/or presentation as defined herein is fused to an encoded antigenic sequence as defined herein.

Accordingly, in particularly preferred embodiments, the RNA described herein comprises at least one coding region encoding an antigenic sequence and an amino acid sequence enhancing antigen processing and/or presentation, said amino acid sequence enhancing antigen processing and/or presentation preferably being fused to the antigenic sequence, more preferably to the C-terminus of the antigenic sequence as described herein.

Amino acid sequences derived from tetanus toxoid of Clostridium tetani may be employed to overcome self-tolerance mechanisms in order to efficiently mount an immune response to self-antigens by providing T cell help during priming.

It is known that tetanus toxoid heavy chain includes epitopes that can bind promiscuously to MHC class II alleles and induce CD4⁺ memory T cells in almost all tetanus vaccinated individuals. In addition, the combination of tetanus toxoid (TT) helper epitopes with tumor-associated antigens is known to improve the immune stimulation compared to application of tumor-associated antigen alone by providing CD4⁺-mediated T-cell help during priming. To reduce the risk of stimulating CD8⁺ T cells with the tetanus sequences which might compete with the intended induction of tumor antigen-specific T cell response, not the whole fragment C of tetanus toxoid is used as it is known to contain CD8⁺ T cell epitopes. Two peptide sequences containing promiscuously binding helper epitopes were selected alternatively to ensure binding to as many MHC class II alleles as possible. Based on the data of the ex vivo studies the well-known epitopes p2 (QYIKANSKFIGITEL; TT₈₃₀₋₈₄₄) and p16 (MTNSVDDALINSTKIYSYFPSVISKVNQGAQG; TT₅₇₈₋₆₀₉) were selected. The p2 epitope was already used for peptide vaccination in clinical trials to boost anti-melanoma activity.

Present non-clinical data (unpublished) showed that RNA vaccines encoding both a tumor antigen plus promiscuously binding tetanus toxoid sequences lead to enhanced CD8⁺ T-cell responses directed against the tumor antigen and improved break of tolerance. Immunomonitoring data from patients vaccinated with vaccines including those sequences fused in frame with the tumor antigen-specific sequences reveal that the tetanus sequences chosen are able to induce tetanus-specific T cell responses in almost all patients.

According to certain embodiments, an amino acid sequence which breaks immunological tolerance is fused, either directly or through a linker, e.g., a linker having the amino acid sequence according to SEQ ID NO: 11, to an antigenic sequence.

Such amino acid sequences which break immunological tolerance are preferably located at the C-terminus of the antigenic sequence (and optionally at the N-terminus of the amino acid sequence enhancing antigen processing and/or presentation, wherein the amino acid sequence which breaks immunological tolerance and the amino acid sequence enhancing antigen processing and/or presentation may be fused either directly or through a linker, e.g., a linker having the amino acid sequence according to SEQ ID NO: 12), without being limited thereto. Amino acid sequences which break immunological tolerance as defined herein preferably improve T cell responses. In one embodiment, the amino acid sequence which breaks immunological tolerance as defined herein includes, without being limited thereto, sequences derived from tetanus toxoid-derived helper sequences p2 and p16 (P2P16), in particular a sequence comprising the amino acid sequence of SEQ ID NO: 10 or a functional variant thereof.

In one embodiment, an amino acid sequence which breaks immunological tolerance comprises the amino acid sequence of SEQ ID NO: 10, an amino acid sequence having at least 99%, 98%, 97%, 96%, 95%, 90%, 85%, or 80% identity to the amino acid sequence of SEQ ID NO: 10, or a functional fragment of the amino acid sequence of SEQ ID NO: 10, or the amino acid sequence having at least 99%, 98%, 97%, 96%, 95%, 90%, 85%, or 80% identity to the amino acid sequence of SEQ ID NO: 10. In one embodiment, an amino acid sequence which breaks immunological tolerance comprises the amino acid sequence of SEQ ID NO: 10.

Instead of using antigen RNAs fused with tetanus toxoid helper epitope, the antigen-coding RNAs may be co-administered with a separate RNA coding for TT helper epitope during vaccination. Here, the TT helper epitope-coding RNA can be added to each of the antigen-coding RNAs before preparation. In this way, mixed lipoplex nanoparticles may be formed comprising both, antigen and helper epitope coding RNA in order to deliver both compounds to a given APC.

Accordingly, the present invention may provide for the use of particles such as lipoplex particles comprising:
(i) RNA encoding a vaccine antigen, and
(ii) RNA encoding an amino acid sequence which breaks immunological tolerance.

In one embodiment, the amino acid sequence which breaks immunological tolerance comprises helper epitopes, preferably tetanus toxoid-derived helper epitopes.

In one embodiment, the RNA encoding a vaccine antigen is co-formulated as particles such as lipoplex particles with the RNA encoding an amino acid sequence which breaks immunological tolerance at a ratio of about 4:1 to about 16:1, about 6:1 to about 14:1, about 8:1 to about 12:1, or about 10:1.

In the following, embodiments of the vaccine RNAs are described, wherein certain terms used when describing elements thereof have the following meanings:

hAg-Kozak: 5′-UTR sequence of the human alpha-globin mRNA with an optimized ‘Kozak sequence’ to increase translational efficiency.

sec/MITD: Fusion-protein tags derived from the sequence encoding the human MHC class I complex (HLA-B51, haplotype A2, B27/B51, Cw2/Cw3), which have been shown to improve antigen processing and presentation. Sec corresponds to the 78 bp fragment coding for the secretory signal peptide, which guides translocation of the nascent polypeptide chain into the endoplasmatic reticulum. MITD corresponds to the transmembrane and cytoplasmic domain of the MHC class I molecule, also called MHC class I trafficking domain.

Antigen: Sequences encoding the respective antigenic sequence.

Glycine-serine linker (GS): Sequences coding for short linker peptides predominantly consisting of the amino acids glycine (G) and serine (S), as commonly used for fusion proteins.

P2P16: Sequence coding for tetanus toxoid-derived helper epitopes to break immunological tolerance.

FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.

A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency.

In one embodiment, vaccine RNA described herein has the structure:
hAg-Kozak-sec-GS(1)-Antigen-GS(2)-P2P16-GS(3)-MITD-FI-A30L70

In one embodiment, vaccine antigen described herein has the structure:
sec-GS(1)-Antigen-GS(2)-P2P16-GS(3)-MITD

In one embodiment, hAg-Kozak comprises the nucleotide sequence of SEQ ID NO: 13. In one embodiment, sec comprises the amino acid sequence of SEQ ID NO: 8. In one embodiment, P2P16 comprises the the amino acid sequence of SEQ ID NO: 10. In one embodiment, MITD comprises the the amino acid sequence of SEQ ID NO: 9. In one embodiment, GS(1) comprises the amino acid sequence of SEQ ID NO: 11. In one embodiment, GS(2) comprises the amino acid sequence of SEQ ID NO: 11. In one embodiment, GS(3) comprises the amino acid sequence of SEQ ID NO: 12. In one embodiment, FI comprises the nucleotide sequence of SEQ ID NO: 14. In one embodiment, A30L70 comprises the nucleotide sequence of SEQ ID NO: 15. The preferred 5′ cap structure is beta-S-ARCA(D1).

### Schedule of Administration of Immunostimulant RNA

The present invention comprises the use of RNA encoding an amino acid sequence comprising a cytokine protein (immunostimulant RNA) at two different doses, i.e., at a first dose and at a second dose. It has been found that administration of a first dose (a pre-dose) of immunostimulant RNA in an amount lower than the amount of a second dose (e.g., a target dose) of the immunostimulant RNA reduces or prevents the level of unwanted response or reaction in a subject. The first dose may be suitable to generate a familiarization/habituation effect and may be lower than a therapeutically effective dose of the immunostimulant RNA. The second dose may be a therapeutically effective amount of the immunostimulant RNA.

In some embodiments, the schedules for administering immunostimulant RNA described herein do not cause an unacceptable level of unwanted response or reaction. In some embodiments, the schedules for administering immunostimulant RNA described herein cause a lower level of unwanted response or reaction, e.g., when compared to only the administration of a second dose described herein without administration of a first dose described herein. In embodiments, the lower level of unwanted response or reaction may involve a decrease in the unwanted response or reaction, e.g., less than 1%, 5%, 10%, 25%, 30%, 35% or 40%, e.g., as measured by one or more assays or symptoms. In some embodiments, the schedules for administering immunostimulant RNA described herein do not cause a detectable level of unwanted response or reaction.

In one embodiment, the amount of said RNA administered in said first dose is no more than 80%, 75%, 50%, 40%, 30%, 25%, 20%, 15%, 10% or 5% of the amount of said RNA administered in said second dose.

In one embodiment, the amount of immunostimulant RNA administered in said first dose is no more than 200 μg, 190 μg, 180 μg, 170 μg, 160 μg, 150 μg, 140 μg, 130 μg, 120 μg, 110 μg, 100 μg, 90 μg, 80 μg, 70 μg, 60 μg, 50 μg, 40 μg, 30 μg, 20 μg, 10 μg, 5 μg, 4 μg, 3 μg, 2 μg, 1 μg, 0.5 μg, 0.4 μg, 0.3 μg, 0.2 μg, or 0.1 μg per kg body weight, and the second dose is greater than said first dose.

In one embodiment, the amount of immunostimulant RNA administered in said second dose is greater than 20 μg, 30 μg, 40 μg, 50 μg, 60 μg, 70 μg, 80 μg, 90 μg, 100 μg, 150 μg, 200 μg, 250 μg, 300 μg, 350 μg, or 400 μg per kg body weight, and the second dose is greater than said first dose.

Immunostimulant RNA of said first dose amount and/or of said second dose amount may be administered more than once, e.g., 2-fold, 3-fold, 4-fold, 5-fold or even more. In one embodiment, the second dose or one of the second doses is not administered prior to completion of administration of the first dose or the first doses. In one embodiment, a second dose/the first of the second doses is administered following administration of the first dose/the final of the first doses.

In one embodiment, more than 1, 2, 3, 4, 5, 6, 7, 14, or 21 days separate the administration of the first dose/completion of the administration of the first dose(s) and the administration of the second dose/initiation of the administration of the second dose(s).

In one embodiment, no more than 56, 49, 42, 35, or 28 days separate the administration of the first dose/completion of the administration of the first dose(s) and the administration of the second dose/initiation of the administration of the second dose(s).

The unwanted response or reaction may involve NK cells and may comprise one or more selected from the group consisting of increase in NK cell number, fever, malaise, reduction of body weight, increase in activity of liver enzymes, capillary leak syndrome, hypotension and edema. In one embodiment, the liver enzymes comprise one or more selected from the group consisting of alanine-aminotransferase (ALAT), aspartate-aminotransferase (ASAT), and lactate-dehydrogenase (LDH). In one embodiment, the unwanted response or reaction involves an induction of NK cell expansion.

According to the disclosure, vaccination, e.g., administration of vaccine RNA, may result in stimulation of T cells by antigenic epitopes. The disclosure further provides for the maintenance and/or further stimulation of the Tcells by administering immunostimulant RNA.

In one embodiment, T cells are antigen-stimulated by tumor-expressed antigen/tumor-presented antigenic epitopes. In this embodiment, no vaccination, i.e., administration of vaccine, may take place. In one embodiment, one or more immune checkpoint inhibitors and/or radiotherapy is also administered.

### Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA.

The nucleic acids described herein may be recombinant and/or isolated molecules.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA. In certain embodiments of the present disclosure, the RNA is “replicon RNA” or simply a “replicon”, in particular “self-replicating RNA” or “self-amplifying RNA”. In one particularly preferred embodiment, the replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In one embodiment, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

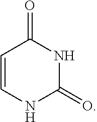

The term “uridine,” as used herein, describes one of the nucleosides that can occur in RNA. The structure of uridine is:

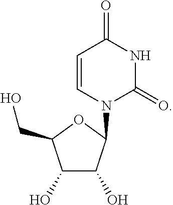 [SMILES: O=c1ccn([C@@H]2O[C@H](CO)[C@@H](O)[C@H]2O)c(=O)[nH]1]

UTP (uridine 5′-triphosphate) has the following structure:

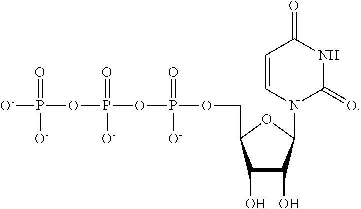

Pseudo-UTP (pseudouridine 5′-triphosphate) has the following structure:

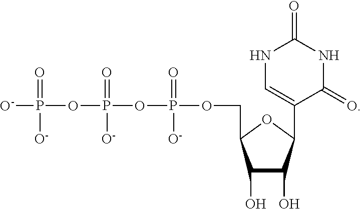

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

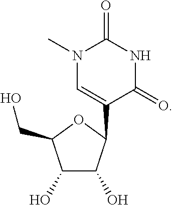 [SMILES: Cn1cc([C@@H]2O[C@H](CO)[C@@H](O)[C@H]2O)c(=O)[nH]c1=O]

N1-methyl-pseudo-UTP has the following structure:

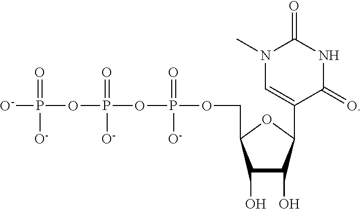

Another exemplary modified nucleoside is 5-methyl-uridine (m5U), which has the structure:

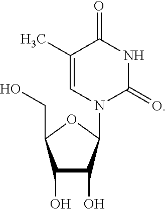

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine.

In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1L). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In one embodiment, the RNA comprises other modified nucleosides or comprises further modified nucleosides, e.g., modified cytidine. For example, in one embodiment, in the RNA 5-methylcytidine is substituted partially or completely, preferably completely, for cytidine. In one embodiment, the RNA comprises 5-methylcytidine and one or more selected from pseudouridine (ψ), N1-methyl-pseudouridine (m1ψ), and 5-methyl-uridine (m5U). In one embodiment, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′- to 5′-triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes. In some embodiments, the building block cap for RNA is m₂^{7,3′-O}Gppp(m₁^{2′-O})ApG (also sometimes referred to as m₂^{7,3′O}G(5′)ppp(5′)m^{2′-O}ApG), which has the following structure:

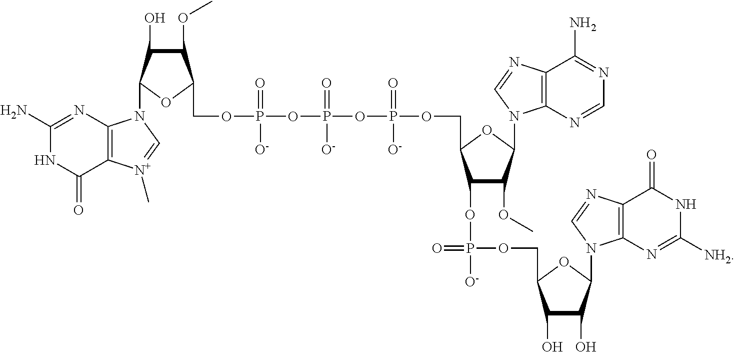

Below is an exemplary Cap1 RNA, which comprises RNA and m₂^{7,3′O}G(5′)ppp(5′)m^{2′-O}ApG:

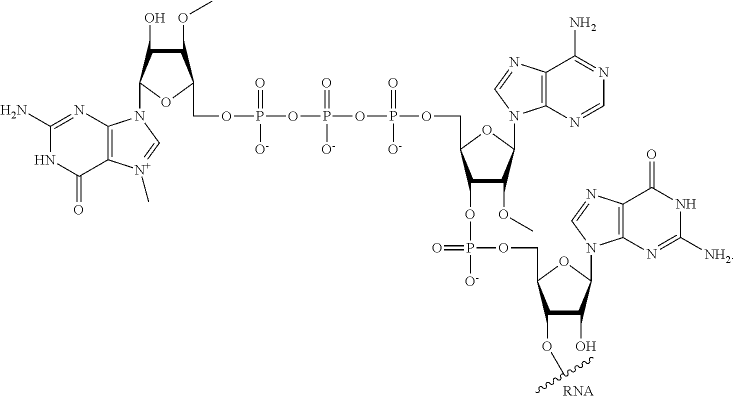

Below is another exemplary Cap1 RNA (no cap analog):

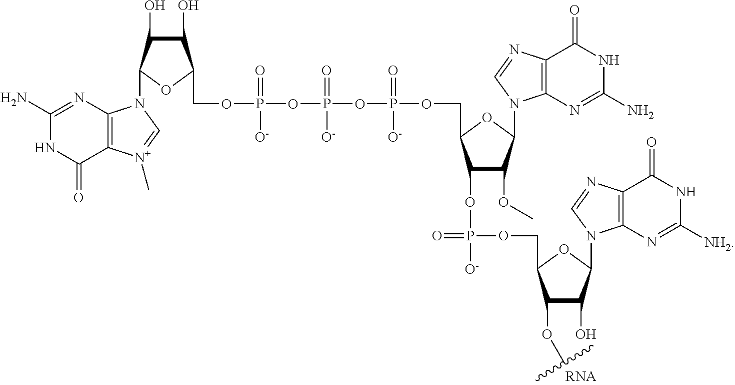

In some embodiments, the RNA is modified with “Cap0” structures using, in one embodiment, the cap analog anti-reverse cap (ARCA Cap (m₂^{7,3′O}G(5′)ppp(5′)G)) with the structure:

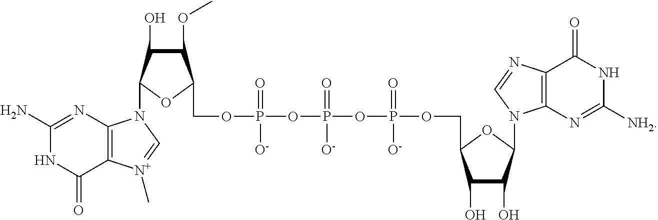

Below is an exemplary Cap0 RNA comprising RNA and m₂^{7,3′O}G(5′)ppp(5′)G:

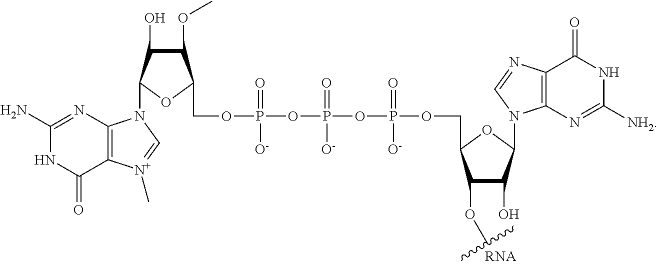

In some embodiments, the “Cap0” structures are generated using the cap analog Beta-S-ARCA (m₂^{7,2′O}G(5′)ppSp(5′)G) with the structure:

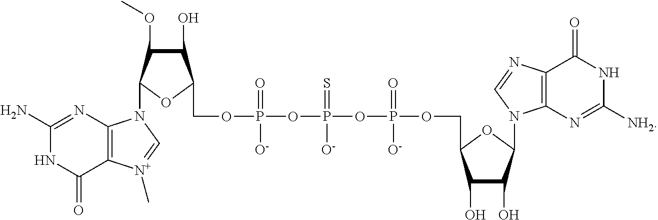

Below is an exemplary Cap0 RNA comprising Beta-S-ARCA (m₂^{7,2′O}G(5′)ppSp(5′)G) and RNA:

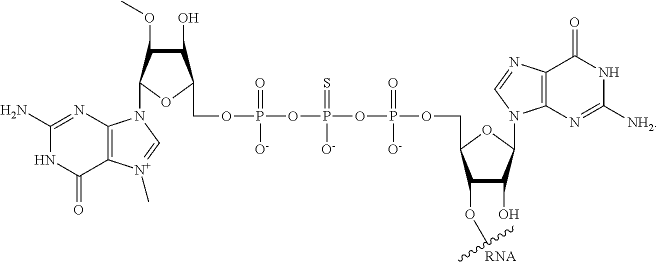

The “D1” diastereomer of beta-S-ARCA or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time (cf., WO 2011/015347, herein incorporated by reference).

A particularly preferred cap is beta-S-ARCA(D1) (m₂^{7,2′-O}GppSpG) or m₂^{7,3′-O}Gppp(m₁^{2′-O})ApG. In one embodiment, in the case of RNA encoding an immostimulant, a preferred cap is m₂^{7,3′-O}Gppp(m₁^{2′-O})ApG. In one embodiment, in the case of RNA encoding a vaccine antigen, a preferred cap is beta-S-ARCA(D1) (m₂^{7,2′-O}GppSpG).

In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g. directly adjacent to the poly(A) sequence.

In some embodiments, RNA comprises a 5′-UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In some embodiments, RNA comprises a 3′-UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

A particularly preferred 5′-UTR comprises the nucleotide sequence of SEQ ID NO: 13. A particularly preferred 3′-UTR comprises the nucleotide sequence of SEQ ID NO: 14.

In some embodiments, the RNA according to the present disclosure comprises a 3′-poly(A) sequence.

As used herein, the term “poly(A) sequence” or “poly-A tail” refers to an uninterrupted or interrupted sequence of adenylate residues which is typically located at the 3′-end of an RNA molecule. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017).

The poly(A) sequence may be of any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly(A) sequence contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, RNA comprises a poly(A) sequence comprising the nucleotide sequence of SEQ ID NO: 15, or a nucleotide sequence having at least 99%, 98%, 97%, 96%, 95%, 90%, 85%, or 80% identity to the nucleotide sequence of SEQ ID NO: 15.

A particularly preferred poly(A) sequence comprises comprises the nucleotide sequence of SEQ ID NO: 15.

According to the disclosure, RNA is preferably administered as single-stranded, 5′-capped mRNA that is translated into the respective protein upon entering cells of a subject being administered the RNA. Preferably, the RNA contains structural elements optimized for maximal efficacy of the RNA with respect to stability and translational efficiency (5′-cap, 5′-UTR, 3′-UTR, poly(A) sequence).

In one embodiment, after administration of the RNA described herein, e.g., formulated as RNA lipid particles, at least a portion of the RNA is delivered to cells of the subject treated. In one embodiment, at least a portion of the RNA is delivered to the cytosol of the cells. In one embodiment, the RNA is translated by the cells to produce the peptide or protein it enodes. In one embodiment of all aspects of the invention, the RNA is transiently expressed in cells of the subject. In one embodiment of all aspects of the invention, the RNA is in vitro transcribed RNA. In one embodiment of all aspects of the invention, in the case of RNA encoding an immunostimulant, the cells are liver cells. In one embodiment, expression of the immostimulant is into the extracellular space, i.e., the immostimulant is secreted. In one embodiment of all aspects of the invention, in the case of vaccine RNA, the cells are spleen cells. In one embodiment of all aspects of the invention, in the case of vaccine RNA, the cells are antigen presenting cells such as professional antigen presenting cells in the spleen. In one embodiment, the cells are dendritic cells or macrophages. In one embodiment, the vaccine sequence is expressed and presented in the context of MHC. RNA particles such as RNA lipid particles described herein may be used for delivering RNA to such cells. For example, lipid nanoparticles (LNP) as described herein may be used for delivering RNA encoding an immunostimulant to liver. For example, lipoplex particles (LPX) as described herein may be used for delivering vaccine RNA to spleen.

In the context of the present disclosure, the term “transcription” relates to a process, wherein the genetic code in a DNA sequence is transcribed into RNA. Subsequently, the RNA may be translated into peptide or protein.

According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”. According to the present invention, the RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

The term “expression” as used herein is defined as the transcription and/or translation of a particular nucleotide sequence.

With respect to RNA, the term “expression” or “translation” relates to the process in the ribosomes of a cell by which a strand of mRNA directs the assembly of a sequence of amino acids to make a peptide or protein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In one embodiment, the RNA to be administered according to the invention is non-immunogenic.

The term “non-immunogenic RNA” as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In one preferred embodiment, non-immunogenic RNA, which is also termed modified RNA (modRNA) herein, is rendered non-immunogenic by incorporating modified nucleosides suppressing RNA-mediated activation of innate immune receptors into the RNA and removing double-stranded RNA (dsRNA).

For rendering the non-immunogenic RNA non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In one embodiment, the modified nucleosides comprises a replacement of one or more uridines with a nucleoside comprising a modified nucleobase. In one embodiment, the modified nucleobase is a modified uracil. In one embodiment, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s2U), α-thio-uridine, 2′-O-methyl-uridine (Urn), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine. In one particularly preferred embodiment, the nucleoside comprising a modified nucleobase is pseudouridine (ψ), N1-methyl-pseudouridine (m1ψ) or 5-methyl-uridine (m5U), in particular N1-methyl-pseudouridine.

In one embodiment, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In one embodiment, an RNA preparation is contacted with a cellulose material and the ssRNA is separated from the cellulose material under conditions which allow binding of dsRNA to the cellulose material and do not allow binding of ssRNA to the cellulose material.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In one embodiment, the removal of dsRNA from non-immunogenic RNA comprises a removal of dsRNA such that less than 10%, less than 5%, less than 4%, less than 3%, less than 2%, less than 1%, less than 0.5%, less than 0.3%, or less than 0.1% of the RNA in the non-immunogenic RNA composition is dsRNA. In one embodiment, the non-immunogenic RNA is free or essentially free of dsRNA. In some embodiments, the non-immunogenic RNA composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

In one embodiment, the non-immunogenic RNA is translated in a cell more efficiently than standard RNA with the same sequence. In one embodiment, translation is enhanced by a factor of 2-fold relative to its unmodified counterpart. In one embodiment, translation is enhanced by a 3-fold factor. In one embodiment, translation is enhanced by a 4-fold factor. In one embodiment, translation is enhanced by a 5-fold factor. In one embodiment, translation is enhanced by a 6-fold factor. In one embodiment, translation is enhanced by a 7-fold factor. In one embodiment, translation is enhanced by an 8-fold factor. In one embodiment, translation is enhanced by a 9-fold factor. In one embodiment, translation is enhanced by a 10-fold factor. In one embodiment, translation is enhanced by a 15-fold factor. In one embodiment, translation is enhanced by a 20-fold factor. In one embodiment, translation is enhanced by a 50-fold factor. In one embodiment, translation is enhanced by a 100-fold factor. In one embodiment, translation is enhanced by a 200-fold factor. In one embodiment, translation is enhanced by a 500-fold factor. In one embodiment, translation is enhanced by a 1000-fold factor. In one embodiment, translation is enhanced by a 2000-fold factor. In one embodiment, the factor is 10-1000-fold. In one embodiment, the factor is 10-100-fold. In one embodiment, the factor is 10-200-fold. In one embodiment, the factor is 10-300-fold. In one embodiment, the factor is 10-500-fold. In one embodiment, the factor is 20-1000-fold. In one embodiment, the factor is 30-1000-fold. In one embodiment, the factor is 50-1000-fold. In one embodiment, the factor is 100-1000-fold. In one embodiment, the factor is 200-1000-fold. In one embodiment, translation is enhanced by any other significant amount or range of amounts.

In one embodiment, the non-immunogenic RNA exhibits significantly less innate immunogenicity than standard RNA with the same sequence. In one embodiment, the non-immunogenic RNA exhibits an innate immune response that is 2-fold less than its unmodified counterpart. In one embodiment, innate immunogenicity is reduced by a 3-fold factor. In one embodiment, innate immunogenicity is reduced by a 4-fold factor. In one embodiment, innate immunogenicity is reduced by a 5-fold factor. In one embodiment, innate immunogenicity is reduced by a 6-fold factor. In one embodiment, innate immunogenicity is reduced by a 7-fold factor. In one embodiment, innate immunogenicity is reduced by a 8-fold factor. In one embodiment, innate immunogenicity is reduced by a 9-fold factor. In one embodiment, innate immunogenicity is reduced by a 10-fold factor. In one embodiment, innate immunogenicity is reduced by a 15-fold factor. In one embodiment, innate immunogenicity is reduced by a 20-fold factor. In one embodiment, innate immunogenicity is reduced by a 50-fold factor. In one embodiment, innate immunogenicity is reduced by a 100-fold factor. In one embodiment, innate immunogenicity is reduced by a 200-fold factor. In one embodiment, innate immunogenicity is reduced by a 500-fold factor. In one embodiment, innate immunogenicity is reduced by a 1000-fold factor. In one embodiment, innate immunogenicity is reduced by a 2000-fold factor.

The term “exhibits significantly less innate immunogenicity” refers to a detectable decrease in innate immunogenicity. In one embodiment, the term refers to a decrease such that an effective amount of the non-immunogenic RNA can be administered without triggering a detectable innate immune response. In one embodiment, the term refers to a decrease such that the non-immunogenic RNA can be repeatedly administered without eliciting an innate immune response sufficient to detectably reduce production of the protein encoded by the non-immunogenic RNA. In one embodiment, the decrease is such that the non-immunogenic RNA can be repeatedly administered without eliciting an innate immune response sufficient to eliminate detectable production of the protein encoded by the non-immunogenic RNA. “Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system. As used herein “endogenous” refers to any material from or produced inside an organism, cell, tissue or system.

As used herein, the term “exogenous” refers to any material introduced from or produced outside an organism, cell, tissue or system.

### Codon-Optimization/Increase in G/C Content

In some embodiment, an amino acid sequence described herein is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In one embodiment, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present invention, coding regions are preferably codon-optimized for optimal expression in a subject to be treated using the RNA molecules described herein. Codon-optimization is based on the finding that the translation efficiency is also determined by a different frequency in the occurrence of tRNAs in cells. Thus, the sequence of RNA may be modified such that codons for which frequently occurring tRNAs are available are inserted in place of “rare codons”.

In some embodiments of the invention, the guanosine/cytosine (G/C) content of the coding region of the RNA described herein is increased compared to the G/C content of the corresponding coding sequence of the wild type RNA, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favourable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the RNA described herein is increased by at least 10%, at least 20%, at least 30%, at least 40%, at least 50%, at least 55%, or even more compared to the G/C content of the coding region of the wild type RNA.

### Nucleic Acid Containing Particles

Nucleic acids such as RNA described herein may be administered formulated as particles.

In the context of the present disclosure, the term “particle” relates to a structured entity formed by molecules or molecule complexes. In one embodiment, the term “particle” relates to a micro- or nano-sized structure, such as a micro- or nano-sized compact structure dispersed in a medium. In one embodiment, a particle is a nucleic acid containing particle such as a particle comprising DNA, RNA or a mixture thereof.

Electrostatic interactions between positively charged molecules such as polymers and lipids and negatively charged nucleic acid are involved in particle formation. This results in complexation and spontaneous formation of nucleic acid particles. In one embodiment, a nucleic acid particle is a nanoparticle.

As used in the present disclosure, “nanoparticle” refers to a particle having an average diameter suitable for parenteral administration.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and/or the cationic polymer combine together with the nucleic acid to form aggregates, and this aggregation results in colloidally stable particles.

In one embodiment, particles described herein further comprise at least one lipid or lipid-like material other than a cationic or cationically ionizable lipid or lipid-like material, at least one polymer other than a cationic polymer, or a mixture thereof

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features,

Nucleic acid particles described herein may have an average diameter that in one embodiment ranges from about 30 nm to about 1000 nm, from about 50 nm to about 800 nm, from about 70 nm to about 600 nm, from about 90 nm to about 400 nm, or from about 100 nm to about 300 nm.

Nucleic acid particles described herein may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

With respect to RNA lipid particles, the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in one embodiment, formed as follows: an ethanol solution comprising lipids, such as cationic lipids and additional lipids, is injected into an aqueous solution under stirring. In one embodiment, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: ionizable cationic lipids, neutral lipids such as phospholipids, a steroid such as cholesterol, and a polymer conjugated lipid such as polyethylene glycol (PEG)-lipids. Each component is responsible for payload protection, and enables effective intracellular delivery. LNPs may be prepared by mixing lipids dissolved in ethanol rapidly with nucleic acid in an aqueous buffer.

The term “average diameter” refers to the mean hydrodynamic diameter of particles as measured by dynamic laser light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells.

Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

In particulate formulation, it is possible that each RNA species (e.g. RNA encoding IL2 immunostimulant and RNA encoding IL7 immunostimulant) is separately formulated as an individual particulate formulation. In that case, each individual particulate formulation will comprise one RNA species. The individual particulate formulations may be present as separate entities, e.g. in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations). In one embodiment, a composition such as a pharmaceutical composition comprises more than one individual particle formulation. Respective pharmaceutical compositions are referred to as mixed particulate formulations. Mixed particulate formulations according to the invention are obtainable by forming, separately, individual particulate formulations, as described above, followed by a step of mixing of the individual particulate formulations. By the step of mixing, a formulation comprising a mixed population of RNA-containing particles is obtainable (for illustration: e.g. a first population of particles may contain RNA encoding IL2 immunostimulant, and a second formulation of particles may contain RNA encoding IL7 immunostimulant). Individual particulate populations may be together in one container, comprising a mixed population of individual particulate formulations. Alternatively, it is possible that all RNA species of the pharmaceutical composition (e.g. RNA encoding IL2 immunostimulant and RNA encoding IL7 immunostimulant) are formulated together as a combined particulate formulation. Such formulations are obtainable by providing a combined formulation (typically combined solution) of all RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

### Cationic Polymer

Given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(S3-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties such as those described herein.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine, in particular protamine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75·10² to 10⁷ Da, preferably 1000 to 10⁵ Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipid and Lipid-Like Material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides. Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids or Lipid-Like Materials

The nucleic acid particles described herein may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances.

In one embodiment, the cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), I,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide ((3AE-DMRIE), N-(4-carboxybenzyl)-N,N-dimethyl-2,3-bis(oleoyloxy)propan-1-aminium (DOBAQ), 2-({8-[(3S3)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

### Additional Lipids or Lipid-Like Materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In certain embodiments, the nucleic acid particles include both a cationic lipid and an additional lipid.

In one embodiment, particles described herein include a polymer conjugated lipid such as a pegylated lipid. The term “pegylated lipid” refers to a molecule comprising both a lipid portion and a polyethylene glycol portion. Pegylated lipids are known in the art.

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

### Lipoplex Particles

In certain embodiments of the present disclosure, the RNA described herein may be present in RNA lipoplex particles.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

### Lipid Nanoparticles (LNPs)

In one embodiment, nucleic acid such as RNA described herein is administered in the form of lipid nanoparticles (LNPs). The LNP may comprise any lipid capable of forming a particle to which the one or more nucleic acid molecules are attached, or in which the one or more nucleic acid molecules are encapsulated.

In one embodiment, the LNP comprises one or more cationic lipids, and one or more stabilizing lipids. Stabilizing lipids include neutral lipids and pegylated lipids.

In one embodiment, the LNP comprises a cationic lipid, a neutral lipid, a steroid, a polymer conjugated lipid; and the RNA, encapsulated within or associated with the lipid nanoparticle.

In one embodiment, the LNP comprises from 40 to 60 mol percent, or from 50 to 60 mol percent of the cationic lipid.

In one embodiment, the neutral lipid is present in a concentration ranging from 5 to 15 mol percent, from 7 to 13 mol percent, or from 9 to 12 mol percent.

In one embodiment, the steroid is present in a concentration ranging from 30 to 50 mol percent, or from 30 to 40 mol percent.

In one embodiment, the LNP comprises from 1 to 10 mol percent, from 1 to 5 mol percent, or from 1 to 2.5 mol percent of the polymer conjugated lipid.

In one embodiment, the LNP comprises from 40 to 60 mol percent a cationic lipid; from 5 to 15 mol percent of a neutral lipid; from 30 to 50 mol percent of a steroid; from 1 to 10 mol percent of a polymer conjugated lipid; and the RNA, encapsulated within or associated with the lipid nanoparticle.

In one embodiment, the mol percent is determined based on total mol of lipid present in the lipid nanoparticle.

In one embodiment, the neutral lipid is selected from the group consisting of DSPC, DPPC, DMPC, DOPC, POPC, DOPE, DOPG, DPPG, POPE, DPPE, DMPE, DSPE, and SM. In one embodiment, the neutral lipid is selected from the group consisting of DSPC, DPPC, DMPC, DOPC, POPC, DOPE and SM. In one embodiment, the neutral lipid is DSPC.

In one embodiment, the steroid is cholesterol.

In one embodiment, the polymer conjugated lipid is a pegylated lipid. In one embodiment, the pegylated lipid has the following structure:

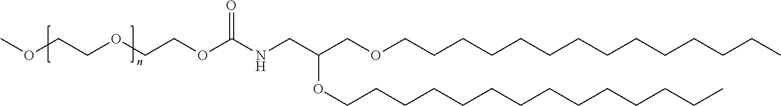

wherein n has a mean value ranging from 30 to 60, such as about 50. In one embodiment, the pegylated lipid is PEG₂₀₀₀-C-DMA.

In one embodiment, the cationic lipid component of the LNPs has the following structure:

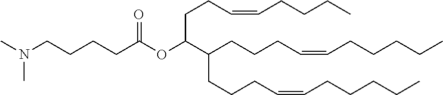 [SMILES: CCCC/C=C\CCC(OC(=O)CCCCN(C)C)C(CCC/C=C\CCCCC)CCC/C=C\CCCCC]

In one embodiment, the cationic lipid is 3D-P-DMA.

In some embodiments, the LNP comprises 3D-P-DMA, RNA, a neutral lipid, a steroid and a pegylated lipid. In some embodiments, the neutral lipid is DSPC. In some embodiments, the steroid is cholesterol. In some embodiments, the pegylated lipid is PEG₂₀₀₀-C-DMA.

In some embodiments, the 3D-P-DMA is present in the LNP in an amount from about 40 to about 60 mole percent. In one embodiment, the neutral lipid is present in the LNP in an amount from about 5 to about 15 mole percent. In one embodiment, the steroid is present in the LNP in an amount from about 30 to about 50 mole percent. In one embodiment, the pegylated lipid such as PEG₂₀₀₀-C-DMA is present in the LNP in an amount from about 1 to about 10 mole percent.

### RNA Targeting

Some aspects of the disclosure involve the targeted delivery of the RNA disclosed herein (e.g., vaccine RNA or immunostimulant RNA).

In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is vaccine RNA.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naïve lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)].

The spleen targeting RNA lipoplex particles described herein at physiological pH preferably have a net negative charge such as a charge ratio of positive charges to negative charges from about 1.9:2 to about 1:2, or about 1.6:2 to about 1:2, or about 1.6:2 to about 1.1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

Immunostimulants such as IL2 and/or IL7 may be provided to a subject by administering to the subject RNA encoding an immunostimulant in a formulation for preferential delivery of RNA to liver or liver tissue. The delivery of RNA to such target organ or tissue is preferred, in particular, if it is desired to express large amounts of the immunostimulant and/or if systemic presence of the immunostimulant, in particular in significant amounts, is desired or required. RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses. Furthermore, lipid nanoparticles (LNPs) as described herein may be used to transport RNA into the liver.

### Immune Checkpoint Inhibitor

As described herein, in one embodiment, the RNA described herein such as vaccine RNA and/or immunostimulant RNA is administered together, i.e., co-administered, with a checkpoint inhibitor to a subject, e.g., a patient. In certain embodiments, the checkpoint inhibitor and the RNA are administered as a single composition to the subject. In certain embodiments, the checkpoint inhibitor and the RNA are administered concurrently (as separate compositions at the same time) to the subject. In certain embodiments, the checkpoint inhibitor and the RNA are administered separately to the subject. In certain embodiments, the checkpoint inhibitor is administered before the RNA to the subject. In certain embodiments, the checkpoint inhibitor is administered after the RNA to the subject. In certain embodiments, the checkpoint inhibitor and the RNA are administered to the subject on the same day. In certain embodiments, the checkpoint inhibitor and the RNA are administered to the subject on different days.

As used herein, “immune checkpoint” refers to regulators of the immune system, and, in particular, co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1 and/or PD-L2. In certain embodiments, the inhibitory signal is the interaction between CTLA-4 and CD80 or CD86 to displace CD28 binding. In certain embodiments the inhibitory signal is the interaction between LAG-3 and MHC class II molecules. In certain embodiments, the inhibitory signal is the interaction between TIM-3 and one or more of its ligands, such as galectin 9, PtdSer, HMGB1 and CEACAM1. In certain embodiments, the inhibitory signal is the interaction between one or several KIRs and their ligands. In certain embodiments, the inhibitory signal is the interaction between TIGIT and one or more of its ligands, PVR, PVRL2 and PVRL3. In certain embodiments, the inhibitory signal is the interaction between CD94/NKG2A and HLA-E. In certain embodiments, the inhibitory signal is the interaction between VISTA and its binding partner(s). In certain embodiments, the inhibitory signal is the interaction between one or more Siglecs and their ligands. In certain embodiments, the inhibitory signal is the interaction between GARP and one or more of it ligands. In certain embodiments, the inhibitory signal is the interaction between CD47 and SIRPα. In certain embodiments, the inhibitory signal is the interaction between PVRIG and PVRL2. In certain embodiments, the inhibitory signal is the interaction between CSF1R and CSF1. In certain embodiments, the inhibitory signal is the interaction between BTLA and HVEM. In certain embodiments, the inhibitory signal is part of the adenosinergic pathway, e.g., the interaction between A2AR and/or A2BR and adenosine, produced by CD39 and CD73. In certain embodiments, the inhibitory signal is the interaction between B7-H3 and its receptor and/or B7-H4 and its receptor. In certain embodiments, the inhibitory signal is mediated by IDO, CD20, NOX or TDO.

The “Programmed Death-1 (PD-1)” receptor refers to an immuno-inhibitory receptor belonging to the CD28 family. PD-1 is expressed predominantly on previously activated T cells in vivo, and binds to two ligands, PD-L1 (also known as B7-H1 or CD274) and PD-L2 (also known as B7-DC or CD273). The term “PD-1” as used herein includes human PD-1 (hPD-1), variants, isoforms, and species homologs of hPD-1, and analogs having at least one common epitope with hPD-1. “Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The term “PD-L2” as used herein includes human PD-L2 (hPD-L2), variants, isoforms, and species homologs of hPD-L2, and analogs having at least one common epitope with hPD-L2. The ligands of PD-1 (PD-L1 and PD-L2) are expressed on the surface of antigen-presenting cells, such as dendritic cells or macrophages, and other immune cells. Binding of PD-1 to PD-L1 or PD-L2 results in downregulation of T cell activation. Cancer cells expressing PD-L1 and/or PD-L2 are able to switch off T cells expressing PD-1 what results in suppression of the anticancer immune response. The interaction between PD-1 and its ligands results in a decrease in tumor infiltrating lymphocytes, a decrease in T cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

“Cytotoxic T Lymphocyte Associated Antigen-4 (CTLA-4)” (also known as CD152) is a T cell surface molecule and is a member of the immunoglobulin superfamily. This protein downregulates the immune system by binding to CD80 (B7-1) and CD86 (B7-2). The term “CTLA-4” as used herein includes human CTLA-4 (hCTLA-4), variants, isoforms, and species homologs of hCTLA-4, and analogs having at least one common epitope with hCTLA-4. CTLA-4 is a homolog of the stimulatory checkpoint protein CD28 with much higher binding affinity for CD80 and CD86. CTLA4 is expressed on the surface of activated T cells and its ligands are expressed on the surface of professional antigen-presenting cells. Binding of CTLA-4 to its ligands prevents the co-stimulatory signal of CD28 and produces an inhibitory signal. Thus, CTLA-4 downregulates T cell activation.

“T cell Immunoreceptor with Ig and ITIM domains” (TIGIT, also known as WUCAM or Vstm3) is an immune receptor on T cells and Natural Killer (NK) cells and binds to PVR (CD155) on DCs, macrophages etc., and PVRL2 (CD112; nectin-2) and PVRL3 (CD113; nectin-3) and regulates T cell-mediated immunity. The term “TIGIT” as used herein includes human TIGIT (hTIGIT), variants, isoforms, and species homologs of hTIGIT, and analogs having at least one common epitope with hTIGIT. The term “PVR” as used herein includes human PVR (hPVR), variants, isoforms, and species homologs of hPVR, and analogs having at least one common epitope with hPVR. The term “PVRL2” as used herein includes human PVRL2 (hPVRL2), variants, isoforms, and species homologs of hPVRL2, and analogs having at least one common epitope with hPVRL2. The term “PVRL3” as used herein includes human PVRL3 (hPVRL3), variants, isoforms, and species homologs of hPVRL3, and analogs having at least one common epitope with hPVRL3.

The “B7 family” refers to inhibitory ligands with undefined receptors. The B7 family encompasses B7-H3 and B7-H4, both upregulated on tumor cells and tumor infiltrating cells. The terms “B7-H3” and “B7-H4” as used herein include human B7-H3 (hB7-H3) and human B7-H4 (hB7-H4), variants, isoforms, and species homologs thereof, and analogs having at least one common epitope with B7-H3 and B7-H4, respectively.

“B and T Lymphocyte Attenuator” (BTLA, also known as CD272) is a TNFR family member expressed in Th1 but not Th2 cells. BTLA expression is induced during activation of T cells and is in particular expressed on surfaces of CD8+ T cells. The term “BTLA” as used herein includes human BTLA (hBTLA), variants, isoforms, and species homologs of hBTLA, and analogs having at least one common epitope with hBTLA. BTLA expression is gradually downregulated during differentiation of human CD8+ T cells to effector cell phenotype. Tumor-specific human CD8+ T cells express high levels of BTLA. BTLA binds to “Herpesvirus entry mediator” (HVEM, also known as TNFRSF14 or CD270) and is involved in T cell inhibition. The term “HVEM” as used herein includes human HVEM (hHVEM), variants, isoforms, and species homologs of hHVEM, and analogs having at least one common epitope with hHVEM. BTLA-HVEM complexes negatively regulate T cell immune responses.

“Killer-cell Immunoglobulin-like Receptors” (KIRs) are receptors for MHC Class I molecules on NK T cells and NK cells that are involved in differentiation between healthy and diseased cells. KIRs bind to human leukocyte antigen (HLA) A, B and C, what suppresses normal immune cell activation. The term “KIRs” as used herein includes human KIRs (hKIRs), variants, isoforms, and species homologs of hKIRs, and analogs having at least one common epitope with a hKIR. The term “HLA” as used herein includes variants, isoforms, and species homologs of HLA, and analogs having at least one common epitope with a HLA. KIR as used herein in particular refers to KIR2DL1, KIR2DL2, and/or KIR2DL3.

“Lymphocyte Activation Gene-3 (LAG-3)” (also known as CD223) is an inhibitory receptor associated with inhibition of lymphocyte activity by binding to MHC class II molecules. This receptor enhances the function of Treg cells and inhibits CD8+ effector T cell function leading to immune response suppression. LAG-3 is expressed on activated T cells, NK cells, B cells and DCs. The term “LAG-3” as used herein includes human LAG-3 (hLAG-3), variants, isoforms, and species homologs of hLAG-3, and analogs having at least one common epitope.

“T Cell Membrane Protein-3 (TIM-3)” (also known as HAVcr-2) is an inhibitory receptor involved in the inhibition of lymphocyte activity by inhibition of Th1 cell responses. Its ligand is galectin 9 (GAL9), which is upregulated in various types of cancers. Other TIM-3 ligands include phosphatidyl serine (PtdSer), High Mobility Group Protein 1 (HMGB1) and Carcinoembryonic Antigen Related Cell Adhesion Molecule 1 (CEACAM1). The term “TIM-3” as used herein includes human TIM3 (hTIM-3), variants, isoforms, and species homologs of hTIM-3, and analogs having at least one common epitope. The term “GAL9” as used herein includes human GAL9 (hGAL9), variants, isoforms, and species homologs of hGAL9, and analogs having at least one common epitope. The term “PdtSer” as used herein includes variants and analogs having at least one common epitope. The term “HMGB1” as used herein includes human HMGB1 (hHMGB1), variants, isoforms, and species homologs of hHMGB1, and analogs having at least one common epitope. The term “CEACAM1” as used herein includes human CEACAM1 (hCEACAM1), variants, isoforms, and species homologs of hCEACAM1, and analogs having at least one common epitope.

“CD94/NKG2A” is an inhibitory receptor predominantly expressed on the surface of natural killer cells and of CD8+ T cells. The term “CD94/NKG2A” as used herein includes human CD94/NKG2A (hCD94/NKG2A), variants, isoforms, and species homologs of hCD94/NKG2A, and analogs having at least one common epitope. The CD94/NKG2A receptor is a heterodimer comprising CD94 and NKG2A. It suppresses NK cell activation and CD8+ T cell function, probably by binding to ligands such as HLA-E. CD94/NKG2A restricts cytokine release and cytotoxic response of natural killer cells (NK cells), Natural Killer T cells (NK-T cells) and T cells (α/β and γ/δ). NKG2A is frequently expressed in tumor infiltrating cells and HLA-E is overexpressed in several cancers.

“Indoleamine 2,3-dioxygenase” (IDO) is a tryptophan catabolic enzyme with immune-inhibitory properties. The term “IDO” as used herein includes human IDO (hIDO), variants, isoforms, and species homologs of hIDO, and analogs having at least one common epitope. IDO is the rate limiting enzyme in tryptophan degradation catalyzing its conversion to kynurenine. Therefore, IDO is involved in depletion of essential amino acids. It is known to be involved in suppression of T and NK cells, generation and activation of Tregs and myeloid-derived suppressor cells, and promotion of tumor angiogenesis. IDO is overexpressed in many cancers and was shown to promote immune system escape of tumor cells and to facilitate chronic tumor progression when induced by local inflammation.

In the “adenosinergic pathway” or “adenosine signaling pathway” as used herein ATP is converted to adenosine by the ectonucleotidases CD39 and CD73 resulting in inhibitory signaling through adenosine binding by one or more of the inhibitory adenosine receptors “Adenosine A2A Receptor” (A2AR, also known as ADORA2A) and “Adenosine A2B Receptor” (A2BR, also known as ADORA2B). Adenosine is a nucleoside with immunosuppressive properties and is present in high concentrations in the tumor microenvironment restricting immune cell infiltration, cytotoxicity and cytokine production. Thus, adenosine signaling is a strategy of cancer cells to avoid host immune system clearance. Adenosine signaling through A2AR and A2BR is an important checkpoint in cancer therapy that is activated by high adenosine concentrations typically present in the tumor microenvironment. CD39, CD73, A2AR and A2BR are expressed by most immune cells, including T cells, invariant natural killer cells, B cells, platelets, mast cells and eosinophils. Adenosine signaling through A2AR and A2BR counteracts T cell receptor mediated activation of immune cells and results in increased numbers of Tregs and decreased activation of DCs and effector T cells. The term “CD39” as used herein includes human CD39 (hCD39), variants, isoforms, and species homologs of hCD39, and analogs having at least one common epitope. The term “CD73” as used herein includes human CD73 (hCD73), variants, isoforms, and species homologs of hCD73, and analogs having at least one common epitope. The term “A2AR” as used herein includes human A2AR (hA2AR), variants, isoforms, and species homologs of hA2AR, and analogs having at least one common epitope. The term “A2BR” as used herein includes human A2BR (hA2BR), variants, isoforms, and species homologs of hA2BR, and analogs having at least one common epitope.

“V-domain Ig suppressor of T cell activation” (VISTA, also known as C10orf54) bears homology to PD-L1 but displays a unique expression pattern restricted to the hematopoietic compartment. The term “VISTA” as used herein includes human VISTA (hVISTA), variants, isoforms, and species homologs of hVISTA, and analogs having at least one common epitope. VISTA induces T cell suppression and is expressed by leukocytes within tumors.

The “Sialic acid binding immunoglobulin type lectin” (Siglec) family members recognize sialic acids and are involved in distinction between “self” and “non-self”. The term “Siglecs” as used herein includes human Siglecs (hSiglecs), variants, isoforms, and species homologs of hSiglecs, and analogs having at least one common epitope with one or more hSiglecs. The human genome contains 14 Siglecs of which several are involved in immunosuppression, including, without limitation, Siglec-2, Siglec-3, Siglec-7 and Siglec-9. Siglec receptors bind glycans containing sialic acid, but differ in their recognition of the linkage regiochemistry and spatial distribution of sialic residues. The members of the family also have distinct expression patterns. A broad range of malignancies overexpress one or more Siglecs.

“CD20” is an antigen expressed on the surface of B and T cells. High expression of CD20 can be found in cancers, such as B cell lymphomas, hairy cell leukemia, B cell chronic lymphocytic leukemia, and melanoma cancer stem cells. The term “CD20” as used herein includes human CD20 (hCD20), variants, isoforms, and species homologs of hCD20, and analogs having at least one common epitope.

“Glycoprotein A repetitions predominant” (GARP) plays a role in immune tolerance and the ability of tumors to escape the patient's immune system. The term “GARP” as used herein includes human GARP (hGARP), variants, isoforms, and species homologs of hGARP, and analogs having at least one common epitope. GARP is expressed on lymphocytes including Treg cells in peripheral blood and tumor infiltrating T cells at tumor sites. It probably binds to latent “transforming growth factor β” (TGF-β). Disruption of GARP signaling in Tregs results in decreased tolerance and inhibits migration of Tregs to the gut and increased proliferation of cytotoxic T cells.

“CD47” is a transmembrane protein that binds to the ligand “signal-regulatory protein alpha” (SIRPα). The term “CD47” as used herein includes human CD47 (hCD47), variants, isoforms, and species homologs of hCD47, and analogs having at least one common epitope with hCD47. The term “SIRPα” as used herein includes human SIRPα (hSIRPα), variants, isoforms, and species homologs of hSIRPα, and analogs having at least one common epitope with hSIRPα. CD47 signaling is involved in a range of cellular processes including apoptosis, proliferation, adhesion and migration. CD47 is overexpressed in many cancers and functions as “don't eat me” signal to macrophages. Blocking CD47 signaling through inhibitory anti-CD47 or anti-SIRPα antibodies enables macrophage phagocytosis of cancer cells and fosters the activation of cancer-specific T lymphocytes.

“Poliovirus receptor related immunoglobulin domain containing” (PVRIG, also known as CD112R) binds to “Poliovirus receptor-related 2” (PVRL2). PVRIG and PVRL2 are overexpressed in a number of cancers. PVRIG expression also induces TIGIT and PD-1 expression and PVRL2 and PVR (a TIGIT ligand) are co-overexpressed in several cancers. Blockade of the PVRIG signaling pathway results in increased T cell function and CD8+ T cell responses and, therefore, reduced immune suppression and elevated interferon responses. The term “PVRIG” as used herein includes human PVRIG (hPVRIG), variants, isoforms, and species homologs of hPVRIG, and analogs having at least one common epitope with hPVRIG. “PVRL2” as used herein includes hPVRL2, as defined above.

The “colony-stimulating factor 1” pathway is another checkpoint that can be targeted according to the disclosure. CSF1R is a myeloid growth factor receptor that binds CSF1. Blockade of the CSF1R signaling can functionally reprogram macrophage responses, thereby enhancing antigen presentation and anti-tumor T cell responses. The term “CSF1R” as used herein includes human CSF1R (hCSF1R), variants, isoforms, and species homologs of hCSF1R, and analogs having at least one common epitope with hCSF1R. The term “CSF1” as used herein includes human CSF1 (hCSF1), variants, isoforms, and species homologs of hCSF1, and analogs having at least one common epitope with hCSF1.

“Nicotinamide adenine dinucleotide phosphate NADPH oxidase” refers to an enzyme of the NOX family of enzymes of myeloid cells that generate immunosuppressive reactive oxygen species (ROS). Five NOX enzymes (NOX1 to NOX5) have been found to be involved in cancer development and immunosuppression. Elevated ROS levels have been detected in almost all cancers and promote many aspects of tumor development and progression. NOX produced ROS dampens NK and T cell functions and inhibition of NOX in myeloid cells improves anti-tumor functions of adjacent NK cells and T cells. The term “NOX” as used herein includes human NOX (hNOX), variants, isoforms, and species homologs of hNOX, and analogs having at least one common epitope with hNOX.

Another immune checkpoint that can be targeted according to the disclosure is the signal mediated by “tryptophan-2,3-dioxygenase” (TDO). TDO represents an alternative route to IDO in tryptophan degradation and is involved in immune suppression. Since tumor cells may catabolize tryptophan via TDO instead of IDO, TDO may represent an additional target for checkpoint blockade. Indeed, several cancer cell lines have been found to upregulate TDO and TDO may complement IDO inhibition. The term “TDO” as used herein includes human TDO (hTDO), variants, isoforms, and species homologs of hTDO, and analogs having at least one common epitope with hTDO.

Many of the immune checkpoints are regulated by interactions between specific receptor and ligand pairs, such as those described above. Thus, immune checkpoint proteins mediate immune checkpoint signaling. For example, checkpoint proteins directly or indirectly regulate T cell activation, T cell proliferation and/or T cell function. Cancer cells often exploit these checkpoint pathways to protect themselves from being attacked by the immune system. Hence, the function of checkpoint proteins, which is modulated according to the present disclosure is typically the regulation of T cell activation, T cell proliferation and/or T cell function. Immune checkpoint proteins thus regulate and maintain self-tolerance and the duration and amplitude of physiological immune responses. Many of the immune checkpoint proteins belong to the B7:CD28 family or to the tumor necrosis factor receptor (TNFR) super family and, by binding to specific ligands, activate signaling molecules that are recruited to the cytoplasmic domain (Suzuki et al., 2016, Jap J Clin Onc, 46:191-203).

As used herein, the term “immune checkpoint modulator” or “checkpoint modulator” refers to a molecule or to a compound that modulates the function of one or more checkpoint proteins. Immune checkpoint modulators are typically able to modulate self-tolerance and/or the amplitude and/or the duration of the immune response. Preferably, the immune checkpoint modulator used according to the present disclosure modulates the function of one or more human checkpoint proteins and is, thus, a “human checkpoint modulator”. In a preferred embodiment, the human checkpoint modulator as used herein is an immune checkpoint inhibitor.

As used herein, “immune checkpoint inhibitor” or “checkpoint inhibitor” refers to a molecule that totally or partially reduces, inhibits, interferes with or negatively modulates one or more checkpoint proteins or that totally or partially reduces, inhibits, interferes with or negatively modulates expression of one or more checkpoint proteins. In certain embodiments, the immune checkpoint inhibitor binds to one or more checkpoint proteins. In certain embodiments, the immune checkpoint inhibitor binds to one or more molecules regulating checkpoint proteins. In certain embodiments, the immune checkpoint inhibitor binds to precursors of one or more checkpoint proteins e.g., on DNA- or RNA-level. Any agent that functions as a checkpoint inhibitor according to the present disclosure can be used.

The term “partially” as used herein means at least 5%, 10%, 15%, 20%, 25%, 30%, 35%, 40%, 45%, 50%, 55%, 60%, 65%, 70%, 75%, 80%, 85%, 90%, 95%, 96%, 97%, 98% or 99% in the level, e.g., in the level of inhibition of a checkpoint protein.

In certain embodiments, the immune checkpoint inhibitor suitable for use in the methods disclosed herein, is an antagonist of inhibitory signals, e.g., an antibody which targets, for example, PD-1, PD-L1, CTLA-4, LAG-3, B7-H3, B7-H4, or TIM-3. These ligands and receptors are reviewed in Pardoll, D., Nature. 12: 252-264, 2012. Further immune checkpoint proteins that can be targeted according the disclosure are described herein.

In certain embodiments, the immune checkpoint inhibitor prevents inhibitory signals associated with the immune checkpoint. In certain embodiments, the immune checkpoint inhibitor is an antibody, or fragment thereof that disrupts inhibitory signaling associated with the immune checkpoint. In certain embodiments, the immune checkpoint inhibitor is a small molecule inhibitor that disrupts inhibitory signaling. In certain embodiments, the immune checkpoint inhibitor is a peptide-based inhibitor that disrupts inhibitory signaling. In certain embodiments, the immune checkpoint inhibitor is an inhibitory nucleic acid molecule that disrupts inhibitory signaling.

In certain embodiments, the immune checkpoint inhibitor is an antibody, fragment thereof, or antibody mimic, that prevents the interaction between checkpoint blocker proteins, e.g., an antibody, or fragment thereof that prevents the interaction between PD-1 and PD-L1 or PD-L2. In certain embodiments, the immune checkpoint inhibitor is an antibody, fragment thereof, or antibody mimic, that prevents the interaction between CTLA-4 and CD80 or CD86. In certain embodiments, the immune checkpoint inhibitor is an antibody, fragment thereof, or antibody mimic, that prevents the interaction between LAG-3 and its ligands, or TIM-3 and its ligands. In certain embodiments, the immune checkpoint inhibitor prevents inhibitory signaling through CD39 and/or CD73 and/or the interaction of A2AR and/or A2BR with adenosine. In certain embodiments, the immune checkpoint inhibitor prevents interaction of B7-H3 with its receptor and/or of B7-H4 with its receptor. In certain embodiments, the immune checkpoint inhibitor prevents the interaction of BTLA with its ligand HVEM. In certain embodiments, the immune checkpoint inhibitor prevents the interaction of one or more KIRs with their respective ligands. In certain embodiments, the immune checkpoint inhibitor prevents the interaction of LAG-3 with one or more of its ligands. In certain embodiments, the immune checkpoint inhibitor prevents the interaction of TIM-3 with one or more of its ligands Galectin-9, PtdSer, HMGB1 and CEACAM1. In certain embodiments, the immune checkpoint inhibitor prevents the interaction of TIGIT with one or more of its ligands PVR, PVRL2 and PVRL3. In certain embodiments, the immune checkpoint inhibitor prevents the interaction of CD94/NKG2A with HLA-E. In certain embodiments, the immune checkpoint inhibitor prevents the interaction of VISTA with one or more of its binding partners. In certain embodiments, the immune checkpoint inhibitor prevents the interaction of one or more Siglecs and their respective ligands. In certain embodiments, the immune checkpoint inhibitor prevents CD20 signaling. In certain embodiments, the immune checkpoint inhibitor prevents the interaction of GARP with one or more of its ligands. In certain embodiments, the immune checkpoint inhibitor prevents the interaction of CD47 with SIRPα. In certain embodiments, the immune checkpoint inhibitor prevents the interaction of PVRIG with PVRL2. In certain embodiments, the immune checkpoint inhibitor prevents the interaction of CSF1R with CSF1. In certain embodiments, the immune checkpoint inhibitor prevents NOX signaling. In certain embodiments, the immune checkpoint inhibitor prevents IDO and/or TDO signaling.

Inhibiting or blocking of inhibitory immune checkpoint signaling, as described herein, results in preventing or reversing immune-suppression and establishment or enhancement of T cell immunity against cancer cells. In one embodiment, inhibition of immune checkpoint signaling, as described herein, reduces or inhibits dysfunction of the immune system. In one embodiment, inhibition of immune checkpoint signaling, as described herein, renders dysfunctional immune cells less dysfunctional. In one embodiment, inhibition of immune checkpoint signaling, as described herein, renders a dysfunctional T cell less dysfunctional.

The term “dysfunction”, as used herein, refers to a state of reduced immune responsiveness to antigenic stimulation. The term includes the common elements of both exhaustion and/or anergy in which antigen recognition may occur, but the ensuing immune response is ineffective to control infection or tumor growth. Dysfunction also includes a state in which antigen recognition is retarded due to dysfunctional immune cells.

The term “dysfunctional”, as used herein, refers to an immune cell that is in a state of reduced immune responsiveness to antigen stimulation. Dysfunctional includes unresponsive to antigen recognition and impaired capacity to translate antigen recognition into downstream T cell effector functions, such as proliferation, cytokine production (e.g., IL-2) and/or target cell killing.

The term “anergy”, as used herein, refers to the state of unresponsiveness to antigen stimulation resulting from incomplete or insufficient signals delivered through the T cell receptor (TCR). T cell anergy can also result upon stimulation with antigen in the absence of co-stimulation, resulting in the cell becoming refractory to subsequent activation by the antigen even in the context of co-stimulation. The unresponsive state can often be overridden by the presence of IL-2. Anergic T cells do not undergo clonal expansion and/or acquire effector functions.

The term “exhaustion”, as used herein, refers to immune cell exhaustion, such as T cell exhaustion as a state of T cell dysfunction that arises from sustained TCR signaling that occurs during many chronic infections and cancer. It is distinguished from anergy in that it arises not through incomplete or deficient signaling, but from sustained signaling. Exhaustion is defined by poor effector function, sustained expression of inhibitory receptors and a transcriptional state distinct from that of functional effector or memory T cells. Exhaustion prevents optimal control of diseases (e.g., infection and tumors). Exhaustion can result from both extrinsic negative regulatory pathways (e.g., immunoregulatory cytokines) as well as cell intrinsic negative regulatory pathways (inhibitory immune checkpoint pathways, such as described herein).

“Enhancing T cell function” means to induce, cause or stimulate a T cell to have a sustained or amplified biological function, or renew or reactivate exhausted or inactive T cells. Examples of enhancing T cell function include increased secretion of γ-interferon from CD8+ T cells, increased proliferation, increased antigen responsiveness (e.g., tumor clearance) relative to such levels before the intervention. In one embodiment, the level of enhancement is as least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 200%, or more. Manners of measuring this enhancement are known to one of ordinary skill in the art.

The immune checkpoint inhibitor may be an inhibitory nucleic acid molecule. The term “inhibitory nucleic acid” or “inhibitory nucleic acid molecule” as used herein refers to a nucleic acid molecule, e.g., DNA or RNA, that totally or partially reduces, inhibits, interferes with or negatively modulates one or more checkpoint proteins. Inhibitory nucleic acid molecules include, without limitation, oligonucleotides, siRNA, shRNA, antisense DNA or RNA molecules, and aptamers (e.g., DNA or RNA aptamers).

The term “oligonucleotide” as used herein refers to a nucleic acid molecule that is able to decrease protein expression, in particular expression of a checkpoint protein, such as the checkpoint proteins described herein. Oligonucleotides are short DNA or RNA molecules, typically comprising from 2 to 50 nucleotides. Oligonucleotides maybe single-stranded or double-stranded. A checkpoint inhibitor oligonucleotide may be an antisense-oligonucleotide. Antisense-oligonucleotides are single-stranded DNA or RNA molecules that are complementary to a given sequence, in particular to a sequence of the nucleic acid sequence (or a fragment thereof) of a checkpoint protein. Antisense RNA is typically used to prevent protein translation of mRNA, e.g., of mRNA encoding a checkpoint protein, by binding to said mRNA. Antisense DNA is typically used to target a specific, complementary (coding or non-coding) RNA. If binding takes place, such a DNA/RNA hybrid can be degraded by the enzyme RNase H. Moreover, morpholino antisense oligonucleotides can be used for gene knockdowns in vertebrates. For example, Kryczek et al., 2006 (J Exp Med, 203:871-81) designed B7-H4-specific morpholinos that specifically blocked B7-H4 expression in macrophages, resulting in increased T cell proliferation and reduced tumor volumes in mice with tumor associated antigen (TAA)-specific T cells.

The terms “siRNA” or “small interfering RNA” or “small inhibitory RNA” are used interchangeably herein and refer to a double-stranded RNA molecule with a typical length of 20-25 base pairs that interferes with expression of a specific gene, such as a gene coding for a checkpoint protein, with a complementary nucleotide sequence. In one embodiment, siRNA interferes with mRNA therefore blocking translation, e.g., translation of an immune checkpoint protein. Transfection of exogenous siRNA may be used for gene knockdown, however, the effect maybe only transient, especially in rapidly dividing cells. Stable transfection may be achieved, e.g., by RNA modification or by using an expression vector. Useful modifications and vectors for stable transfection of cells with siRNA are known in the art. siRNA sequences may also be modified to introduce a short loop between the two strands resulting in a “small hairpin RNA” or “shRNA”. shRNA can be processed into a functional siRNA by Dicer. shRNA has a relatively low rate of degradation and turnover. Accordingly, the immune checkpoint inhibitor may be a shRNA.

The term “aptamer” as used herein refers to a single-stranded nucleic acid molecule, such as DNA or RNA, typically in a length of 25-70 nucleotides that is capable of binding to a target molecule, such as a polypeptide. In one embodiment, the aptamer binds to an immune checkpoint protein such as the immune checkpoint proteins described herein. For example, an aptamer according to the disclosure can specifically bind to an immune checkpoint protein or polypeptide, or to a molecule in a signaling pathway that modulates the expression of an immune checkpoint protein or polypeptide. The generation and therapeutic use of aptamers is well known in the art (see, e.g., U.S. Pat. No. 5,475,096).

The terms “small molecule inhibitor” or “small molecule” are used interchangeably herein and refer to a low molecular weight organic compound, usually up to 1000 daltons, that totally or partially reduces, inhibits, interferes with, or negatively modulates one or more checkpoint proteins as described above. Such small molecular inhibitors are usually synthesized by organic chemistry, but may also be isolated from natural sources, such as plants, fungi, and microbes. The small molecular weight allows a small molecule inhibitor to rapidly diffuse across cell membranes. For example, various A2AR antagonists known in the art are organic compounds having a molecular weight below 500 daltons.

The immune checkpoint inhibitor may be an antibody, an antigen-binding fragment thereof, an antibody mimic or a fusion protein comprising an antibody portion with an antigen-binding fragment of the required specificity. Antibodies or antigen-binding fragments thereof are as described herein. Antibodies or antigen-binding fragments thereof that are immune checkpoint inhibitors include in particular antibodies or antigen-binding fragments thereof that bind to immune checkpoint proteins, such as immune checkpoint receptors or immune checkpoint receptor ligands. Antibodies or antigen-binding fragments may also be conjugated to further moieties, as described herein. In particular, antibodies or antigen-binding fragments thereof are chimerized, humanized or human antibodies. Preferably, immune checkpoint inhibitor antibodies or antigen-binding fragments thereof are antagonists of immune checkpoint receptors or of immune checkpoint receptor ligands.

In a preferred embodiment, an antibody that is an immune checkpoint inhibitor, is an isolated antibody.

The antibody that is an immune checkpoint inhibitor or the antigen-binding fragment thereof according to the present disclosure may also be an antibody that cross-competes for antigen binding with any known immune checkpoint inhibitor antibody. In certain embodiments, an immune checkpoint inhibitor antibody cross-competes with one or more of the immune checkpoint inhibitor antibodies described herein. The ability of antibodies to cross-compete for binding to an antigen indicates that these antibodies may bind to the same epitope region of the antigen or when binding to another epitope sterically hinder the binding of known immune checkpoint inhibitor antibodies to that particular epitope region. These cross-competing antibodies may have functional properties very similar to those they are cross-competing with as they are expected to block binding of the immune checkpoint to its ligand either by binding to the same epitope or by sterically hindering the binding of the ligand. Cross-competing antibodies can be readily identified based on their ability to cross-compete with one or more of known antibodies in standard binding assays such as Surface Plasmon Resoncance analysis, ELISA assays or flow cytometry (see, e.g., WO 2013/173223).

In certain embodiments, antibodies or antigen binding fragments thereof that cross-compete for binding to a given antigen with, or bind to the same epitope region of a given antigen as, one or more known antibodies are monoclonal antibodies. For administration to human patients, these cross-competing antibodies can be chimeric antibodies, or humanized or human antibodies. Such chimeric, humanized or human monoclonal antibodies can be prepared and isolated by methods well known in the art.

The checkpoint inhibitor may also be in the form of the soluble form of the molecules (or variants thereof) themselves, e.g., a soluble PD-L1 or PD-L1 fusion.

In the context of the disclosure, more than one checkpoint inhibitor can be used, wherein the more than one checkpoint inhibitors are targeting distinct checkpoint pathways or the same checkpoint pathway. Preferably, the more than one checkpoint inhibitors are distinct checkpoint inhibitors. Preferably, if more than one distinct checkpoint inhibitor is used, in particular at least 2, 3, 4, 5, 6, 7, 8, 9 or 10 distinct checkpoint inhibitors are used, preferably 2, 3, 4 or 5 distinct checkpoint inhibitors are used, more preferably 2, 3 or 4 distinct checkpoint inhibitors are used, even more preferably 2 or 3 distinct checkpoint inhibitors are used and most preferably 2 distinct checkpoint inhibitors are used. Preferred examples of combinations of distinct checkpoint inhibitors include combination of an inhibitor of PD-1 signaling and an inhibitor of CTLA-4 signaling, an inhibitor of PD-1 signaling and an inhibitor of TIGIT signaling, an inhibitor of PD-1 signaling and an inhibitor of B7-H3 and/or B7-H4 signaling, an inhibitor of PD-1 signaling and an inhibitor of BTLA signaling, an inhibitor of PD-1 signaling and an inhibitor of KIR signaling, an inhibitor of PD-1 signaling and an inhibitor of LAG-3 signaling, an inhibitor of PD-1 signaling and an inhibitor of TIM-3 signaling, an inhibitor of PD-1 signaling and an inhibitor of CD94/NKG2A signaling, an inhibitor of PD-1 signaling and an inhibitor of IDO signaling, an inhibitor of PD-1 signaling and an inhibitor of adenosine signaling, an inhibitor of PD-1 signaling and an inhibitor of VISTA signaling, an inhibitor of PD-1 signaling and an inhibitor of Siglec signaling, an inhibitor of PD-1 signaling and an inhibitor of CD20 signaling, an inhibitor of PD-1 signaling and an inhibitor of GARP signaling, an inhibitor of PD-1 signaling and an inhibitor of CD47 signaling, an inhibitor of PD-1 signaling and an inhibitor of PVRIG signaling, an inhibitor of PD-1 signaling and an inhibitor of CSF1R signaling, an inhibitor of PD-1 signaling and an inhibitor of NOX signaling, and an inhibitor of PD-1 signaling and an inhibitor of TDO signaling.

In certain embodiments, the inhibitory immunoregulator (immune checkpoint blocker) is a component of the PD-1/PD-L1 or PD-1/PD-L2 signaling pathway. Accordingly, certain embodiments of the disclosure provide for administering to a subject a checkpoint inhibitor of the PD-1 signaling pathway. In certain embodiments, the checkpoint inhibitor of the PD-1 signaling pathway is a PD-1 inhibitor. In certain embodiments, the checkpoint inhibitor of the PD-1 signaling pathway is a PD-1 ligand inhibitor, such as a PD-L1 inhibitor or a PD-L2 inhibitor. In a preferred embodiment, the checkpoint inhibitor of the PD-1 signaling pathway is an antibody or an antigen-binding portion thereof that disrupts the interaction between the PD-1 receptor and one or more of its ligands, PD-L1 and/or PD-L2. Antibodies which bind to PD-1 and disrupt the interaction between PD-1 and one or more of its ligands are known in the art. In certain embodiments, the antibody or antigen-binding portion thereof binds specifically to PD-1. In certain embodiments, the antibody or antigen-binding portion thereof binds specifically to PD-L1 and inhibits its interaction with PD-1, thereby increasing immune activity. In certain embodiments, the antibody or antigen-binding portion thereof binds specifically to PD-L2 and inhibits its interaction with PD-1, thereby increasing immune activity.

In certain embodiments, the inhibitory immunoregulator is a component of the CTLA-4 signaling pathway. Accordingly, certain embodiments of the disclosure provide for administering to a subject a checkpoint inhibitor of the CTLA-4 signaling pathway. In certain embodiments, the checkpoint inhibitor of the CTLA-4 signaling pathway is a CTLA-4 inhibitor. In certain embodiments, the checkpoint inhibitor of the CTLA-4 signaling pathway is a CTLA-4 ligand inhibitor.

In certain embodiments, the inhibitory immunoregulator is a component of the TIGIT signaling pathway. Accordingly, certain embodiments of the disclosure provide for administering to a subject a checkpoint inhibitor of the TIGIT signaling pathway. In certain embodiments, the checkpoint inhibitor of the TIGIT signaling pathway is a TIGIT inhibitor. In certain embodiments, the checkpoint inhibitor of the TIGIT signaling pathway is a TIGIT ligand inhibitor.

In certain embodiments, the inhibitory immunoregulator is a component of the B7 family signaling pathway. In certain embodiments, the B7 family members are B7-H3 and B7-H4. Certain embodiments of the disclosure provide for administering to a subject a checkpoint inhibitor of B7-H3 and/or B7-4. Accordingly, certain embodiments of the disclosure provide for administering to a subject an antibody or an antigen-binding portion thereof that targets B7-H3 or B7-H4. The B7 family does not have any defined receptors but these ligands are upregulated on tumor cells or tumor-infiltrating cells. Preclinical mouse models have shown that blockade of these ligands can enhance anti-tumor immunity.

In certain embodiments, the inhibitory immunoregulator is a component of the BTLA signaling pathway. Accordingly, certain embodiments of the disclosure provide for administering to a subject a checkpoint inhibitor of the BTLA signaling pathway. In certain embodiments, the checkpoint inhibitor of the BTLA signaling pathway is a BTLA inhibitor. In certain embodiments, the checkpoint inhibitor of the BTLA signaling pathway is a HVEM inhibitor.

In certain embodiments, the inhibitory immunoregulator is a component of one or more KIR signaling pathways. Accordingly, certain embodiments of the disclosure provide for administering to a subject a checkpoint inhibitor of one or more KIR signaling pathways. In certain embodiments, the checkpoint inhibitor of one or more KIR signaling pathways is a KIR inhibitor. In certain embodiments, the checkpoint inhibitor one or more KIR signaling pathways is a KIR ligand inhibitor. For example, the KIR inhibitor according to the present disclosure may be an anti-KIR antibody that binds to KIR2DL1, KIR2DL2, and/or KIR2DL3.

In certain embodiments, the inhibitory immunoregulator is a component of the LAG-3 signaling pathway. Accordingly, certain embodiments of the disclosure provide for administering to a subject a checkpoint inhibitor of LAG-3 signaling. In certain embodiments, the checkpoint inhibitor of the LAG-3 signaling pathway is a LAG-3 inhibitor. In certain embodiments, the checkpoint inhibitor of the LAG-3 signaling pathway is a LAG-3 ligand inhibitor.

In certain embodiments, the inhibitory immunoregulator is a component of the TIM-3 signaling pathway. Accordingly, certain embodiments of the disclosure provide for administering to a subject a checkpoint inhibitor of the TIM-3 signaling pathway. In certain embodiments, the checkpoint inhibitor of the TIM-3 signaling pathway is a TIM-3 inhibitor. In certain embodiments, the checkpoint inhibitor of the TIM-3 signaling pathway is a TIM-3 ligand inhibitor.

In certain embodiments, the inhibitory immunoregulator is a component of the CD94/NKG2A signaling pathway. Accordingly, certain embodiments of the disclosure provide for administering to a subject a checkpoint inhibitor of the CD94/NKG2A signaling pathway. In certain embodiments, the checkpoint inhibitor of the CD94/NKG2A signaling pathway is a CD94/NKG2A inhibitor. In certain embodiments, the checkpoint inhibitor of the CD94/NKG2A signaling pathway is a CD94/NKG2A ligand inhibitor.

In certain embodiments, the inhibitory immunoregulator is a component of the IDO signaling pathway. Accordingly, certain embodiments of the disclosure provide for administering to a subject a checkpoint inhibitor of the IDO signaling pathway, e.g., an IDO inhibitor.

In certain embodiments, the inhibitory immunoregulator is a component of the adenosine signaling pathway. Accordingly, certain embodiments of the disclosure provide for administering to a subject a checkpoint inhibitor of the adenosine signaling pathway. In certain embodiments, the checkpoint inhibitor of the adenosine signaling pathway is a CD39 inhibitor. In certain embodiments, the checkpoint inhibitor of the adenosine signaling pathway is a CD73 inhibitor. In certain embodiments, the checkpoint inhibitor of the adenosine signaling pathway is an A2AR inhibitor. In certain embodiments, the checkpoint inhibitor of the adenosine signaling pathway is an A2BR inhibitor.

In certain embodiments, the inhibitory immunoregulator is a component of the VISTA signaling pathway. Accordingly, certain embodiments of the disclosure provide for administering to a subject a checkpoint inhibitor of the VISTA signaling pathway. In certain embodiments, the checkpoint inhibitor of the VISTA signaling pathway is a VISTA inhibitor.

In certain embodiments, the inhibitory immunoregulator is a component of one or more Siglec signaling pathways. Accordingly, certain embodiments of the disclosure provide for administering to a subject a checkpoint inhibitor of one or more Siglec signaling pathways. In certain embodiments, the checkpoint inhibitor of one or more Siglec signaling pathways is a Siglec inhibitor. In certain embodiments, the checkpoint inhibitor of one or more Siglec signaling pathways is a Siglec ligand inhibitor.

In certain embodiments, the inhibitory immunoregulator is a component of the CD20 signaling pathway. Accordingly, certain embodiments of the disclosure provide for administering to a subject a checkpoint inhibitor of the CD20 signaling pathway. In certain embodiments, the checkpoint inhibitor of the CD20 signaling pathway is a CD20 inhibitor.

In certain embodiments, the inhibitory immunoregulator is a component of the GARP signaling pathway. Accordingly, certain embodiments of the disclosure provide for administering to a subject a checkpoint inhibitor of the GARP signaling pathway. In certain embodiments, the checkpoint inhibitor of the GARP signaling pathway is a GARP inhibitor.

In certain embodiments, the inhibitory immunoregulator is a component of the CD47 signaling pathway. Accordingly, certain embodiments of the disclosure provide for administering to a subject a checkpoint inhibitor of the CD47 signaling pathway. In certain embodiments, the checkpoint inhibitor of the CD47 signaling pathway is a CD47 inhibitor. In certain embodiments, the checkpoint inhibitor of the CD47 signaling pathway is a SIRPα inhibitor.

In certain embodiments, the inhibitory immunoregulator is a component of the PVRIG signaling pathway. Accordingly, certain embodiments of the disclosure provide for administering to a subject a checkpoint inhibitor of the PVRIG signaling pathway. In certain embodiments, the checkpoint inhibitor of the PVRIG signaling pathway is a PVRIG inhibitor. In certain embodiments, the checkpoint inhibitor of the PVRIG signaling pathway is a PVRIG ligand inhibitor.

In certain embodiments, the inhibitory immunoregulator is a component of the CSF1R signaling pathway. Accordingly, certain embodiments of the disclosure provide for administering to a subject a checkpoint inhibitor of the CSF1R signaling pathway. In certain embodiments, the checkpoint inhibitor of the CSF1R signaling pathway is a CSF1R inhibitor. In certain embodiments, the checkpoint inhibitor of the CSF1R signaling pathway is a CSF1 inhibitor.

In certain embodiments, the inhibitory immunoregulator is a component of the NOX signaling pathway. Accordingly, certain embodiments of the disclosure provide for administering to a subject a checkpoint inhibitor of the NOX signaling pathway, e.g., a NOX inhibitor.

In certain embodiments, the inhibitory immunoregulator is a component of the TDO signaling pathway. Accordingly, certain embodiments of the disclosure provide for administering to a subject a checkpoint inhibitor of the TDO signaling pathway, e.g., a TDO inhibitor.

Exemplary PD-1 inhibitors include, without limitation, anti-PD-1 antibodies such as BGB-A317 (BeiGene; see U.S. Pat. No. 8,735,553, WO 2015/35606 and US 2015/0079109), cemiplimab (Regeneron; see WO 2015/112800) and lambrolizumab (e.g., disclosed as hPD109A and its humanized derivatives h409A1, h409A16 and h409A17 in WO2008/156712), AB137132 (Abcam), EH12.2H7 and RMP1-14 (#BE0146; Bioxcell Lifesciences Pvt. LTD.), MIH4 (Affymetrix eBioscience), nivolumab (OPDIVO, BMS-936558; Bristol Myers Squibb; see WO 2006/121168), pembrolizumab (KEYTRUDA; MK-3475; Merck; see WO 2008/156712), pidilizumab (CT-011; CureTech; see Hardy et al., 1994, Cancer Res., 54(22):5793-6 and WO 2009/101611), PDR001 (Novartis; see WO 2015/112900), MEDI0680 (AMP-514; AstraZeneca; see WO 2012/145493), TSR-042 (see WO 2014/179664), REGN-2810 (H4H7798N; cf. US 2015/0203579), JS001 (TAIZHOU JUNSHI PHARMA; see Si-Yang Liu et al., 2007, J. Hematol. Oncol. 70: 136), AMP-224 (GSK-2661380; cf. Li et al., 2016, Int J Mol Sci 17(7):1151 and WO 2010/027827 and WO 2011/066342), PF-06801591 (Pfizer), BGB-A317 (BeiGene; see WO 2015/35606 and US 2015/0079109), BI 754091, SHR-1210 (see WO2015/085847), and antibodies 17D8, 2D3, 4H1, 4A11, 7D3, and 5F4 as described in WO 2006/121168, INCSHR1210 (Jiangsu Hengrui Medicine; also known as SHR-1210; see WO 2015/085847), TSR-042 (Tesaro Biopharmaceutical; also known as ANB011; see WO2014/179664), GLS-010 (Wuxi/Harbin Gloria Pharmaceuticals; also known as WBP3055; see Si-Yang et al., 2017, J. Hematol. Oncol. 70: 136), STI-1110 (Sorrento Therapeutics; see WO 2014/194302), AGEN2034 (Agenus; see WO 2017/040790), MGA012 (Macrogenics; see WO 2017/19846), 1B1308 (Innovent; see WO 2017/024465, WO 2017/025016, WO 2017/132825, and WO 2017/133540), anti-PD-1 antibodies as described, e.g., in U.S. Pat. Nos. 7,488,802, 8,008,449, 8,168,757, WO 03/042402, WO 2010/089411 (further disclosing anti-PD-L1 antibodies), WO 2010/036959, WO 2011/159877 (further disclosing antibodies against TIM-3), WO 2011/082400, WO 2011/161699, WO 2009/014708, WO 03/099196, WO 2009/114335, WO 2012/145493 (further disclosing antibodies against PD-L1), WO 2015/035606, WO 2014/055648 (further disclosing anti-KIR antibodies), US 2018/0185482 (further disclosing anti-PD-L1 and anti-TIGIT antibodies), U.S. Pat. Nos. 8,008,449, 8,779,105, 6,808,710, 8,168,757, US 2016/0272708, and U.S. Pat. No. 8,354,509, small molecule antagonists to the PD-1 signaling pathway as disclosed, e.g., in Shaabani et al., 2018, Expert Op Ther Pat., 28(9):665-678 and Sasikumar and Ramachandra, 2018, BioDrugs, 32(5):481-497, siRNAs directed to PD-1 as disclosed, e.g., in WO 2019/000146 and WO 2018/103501, soluble PD-1 proteins as disclosed in WO 2018/222711 and oncolytic viruses comprising a soluble form of PD-1 as described, e.g., in WO 2018/022831.

In a certain embodiment, the PD-1 inhibitor is nivolumab (OPDIVO; BMS-936558), pembrolizumab (KEYTRUDA; MK-3475), pidilizumab (CT-011), PDR001, MEDI0680 (AMP-514), TSR-042, REGN2810, JS001, AMP-224 (GSK-2661380), PF-06801591, BGB-A317, BI 754091, or SHR-1210.

Exemplary PD-1 ligand inhibitors are PD-L1 inhibitors and PD-L2 inhibitors and include, without limitation, anti-PD-L1 antibodies such as MEDI4736 (durvalumab; AstraZeneca; see WO 2011/066389), MSB-0010718C (see US 2014/0341917), YW243.55.S70 (see SEQ ID NO: 20 of WO 2010/077634 and U.S. Pat. No. 8,217,149), MIH1 (Affymetrix eBioscience; cf. EP 3 230 319), MDX-1105 (Roche/Genentech; see WO2013019906 and U.S. Pat. No. 8,217,149) STI-1014 (Sorrento; see WO2013/181634), CK-301 (Checkpoint Therapeutics), KN035 (3D Med/Alphamab; see Zhang et al., 2017, Cell Discov. 3:17004), atezolizumab (TECENTRIQ; RG7446; MPDL3280A; R05541267; see U.S. Pat. No. 9,724,413), BMS-936559 (Bristol Myers Squibb; see U.S. Pat. No. 7,943,743, WO 2013/173223), avelumab (bavencio; cf. US 2014/0341917), LY3300054 (Eli Lilly Co.), CX-072 (Proclaim-CX-072; also called CytomX; see WO2016/149201), FAZ053, KN035 (see WO2017020801 and WO2017020802), MDX-1105 (see US 2015/0320859), anti-PD-L1 antibodies disclosed in U.S. Pat. No. 7,943,743, including 3G10, 12A4 (also referred to as BMS-936559), 10A5, 5F8, 10H10, 1B12, 7H1, 11E6, 12B7, and 13G4, anti-PD-L1 antibodies as described in WO 2010/077634, U.S. Pat. No. 8,217,149, WO 2010/036959, WO 2010/077634, WO 2011/066342, U.S. Pat. Nos. 8,217,149, 7,943,743, WO 2010/089411, U.S. Pat. Nos. 7,635,757, 8,217,149, US 2009/0317368, WO 2011/066389, WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO 2018/222711, WO2015/112805, WO2015/061668, WO2014/159562, WO2014/165082, WO2014/100079.

Exemplary CTLA-4 inhibitors include, without limitation, the monoclonal antibodies ipilimumab (Yervoy; Bristol Myers Squibb) and tremelimumab (Pfizer/MedImmune), trevilizumab, AGEN-1884 (Agenus) and ATOR-1015, the anti-CTLA4 antibodies disclosed in WO 2001/014424, US 2005/0201994, EP 1212422, U.S. Pat. Nos. 5,811,097, 5,855,887, 6,051,227, 6,682,736, 6,984,720, WO 01/14424, WO 00/37504, US 2002/0039581, US 2002/086014, WO 98/42752, U.S. Pat. Nos. 6,207,156, 5,977,318, 7,109,003, and 7,132,281, the dominant negative proteins abatacept (Orencia; see EP 2 855 533), which comprises the Fe region of IgG 1 fused to the CTLA-4 ECD, and belatacept (Nulojix; see WO 2014/207748), a second generation higher-affinity CTLA-4-Ig variant with two amino acid substitutions in the CTLA-4 ECD relative to abatacept, soluble CTLA-4 polypeptides, e.g., RG2077 and CTLA4-IgG4m (see U.S. Pat. No. 6,750,334), anti-CTLA-4 aptamers and siRNAs directed to CTLA-4, e.g., as disclosed in US 2015/203848. Exemplary CTLA-4 ligand inhibitors are described in Pile et al., 2015 (Encyclopedia of Inflammatory Diseases, M. Parnham (ed.), doi: 10.1007/978-3-0348-0620-6_20).

Exemplary checkpoint inhibitors of the TIGIT signaling pathway include, without limitation, anti-TIGIT antibodies, such as BMS-986207, COM902 (CGEN-15137; Compugen), AB154 (Arcus Biosciences) or etigilimab (OMP-313M32; OncoMed Pharmaceuticals), or the antibodies disclosed in WO2017/059095, in particular “MAB10”, US 2018/0185482, WO 2015/009856, and US 2019/0077864.

Exemplary checkpoint inhibitors of B7-H3 include, without limitation, the Fc-optimized monoclonal antibody enoblituzumab (MGA271; Macrogenics; see US 2012/0294796) and the anti-B7-H3 antibodies MGD009 (Macrogenics) and pidilizumab (see U.S. Pat. No. 7,332,582).

Exemplary B7-H4 inhibitors include, without limitation, antibodies as described in Dangaj et al., 2013 (Cancer Research 73:4820-9) and in Smith et al., 2014 (Gynecol Oncol, 134:181-189), WO 2013/025779 (e.g., 2D1 encoded by SEQ ID NOs: 3 and 4, 2H9 encoded by SEQ ID NO: 37 and 39, and 2E11 encoded by SEQ ID NOs: 41 and 43) and in WO 2013/067492 (e.g., an antibody with an amino acid sequence selected from SEQ ID NOs: 1-8), morpholino antisense oligonucleotides, e.g., as described by Kryczek et al., 2006 (J Exp Med, 203:871-81), or soluble recombinant forms of B7-H4, such as disclosed in US 2012/0177645.

Exemplary BTLA inhibitors include, without limitation, the anti-BTLA antibodies described in Crawford and Wherry, 2009 (J Leukocyte Biol 86:5-8), WO 2011/014438 (e.g., 4C7 or an antibody comprising heavy and light chains according to SEQ ID NOs: 8 and 15 and/or SEQ ID NOs: 11 and 18), WO 2014/183885 (e.g., the antibody deposited under the number CNCM I-4752) and US 2018/155428.

Checkpoint inhibitors of KIR signaling include, without limitation, the monoclonal antibodies lirilumab (1-7F9; IPH2102; see U.S. Pat. No. 8,709,411), IPH4102 (Innate Pharma; see Marie-Cardine et al., 2014, Cancer 74(21): 6060-70), anti-KIR antibodies as disclosed, e.g., in US 2018/208652, US 2018/117147, US 2015/344576, WO 2005/003168, WO 2005/009465, WO 2006/072625, WO 2006/072626, WO 2007/042573, WO 2008/084106 (e.g., an antibody comprising heavy and light chains according to SEQ ID NOs: 2 and 3), WO 2010/065939, WO 2012/071411, WO 2012/160448 and WO 2014/055648.

LAG-3 inhibitors include, without limitation, the anti-LAG-3 antibodies BMS-986016 (Bristol-Myers Squibb; see WO 2014/008218 and WO 2015/116539), 25F7 (see US2011/0150892), IMP731 (see WO 2008/132601), H5L7BW (cf. WO2014140180), MK-4280 (28G-10; Merck; see WO 2016/028672), REGN3767 (Regneron/Sanofi), BAP050 (see WO 2017/019894), IMP-701 (LAG-525; Novartis) Sym022 (Symphogen), TSR-033 (Tesaro), MGD013 (a bispecific DART antibody targeting LAG-3 and PD-1 developed by MacroGenics), B1754111 (Boehringer Ingelheim), FS118 (a bispecific antibody targeting LAG-3 and PD-1 developed by F-star), GSK2831781 (GSK) and antibodies as disclosed in WO 2009/044273, WO 2008/132601, WO 2015/042246, EP 2 320 940, US 2019/169294, US 2019/169292, WO 2016/028672, WO 2016/126858, WO 2016/200782, WO 2015/200119, WO 2017/220569, WO 2017/087589, WO 2017/219995, WO 2017/019846, WO 2017/106129, WO 2017/062888, WO 2018/071500, WO 2017/087901, US 2017/0260271, WO 2017/198741, WO2017/220555, WO2017/015560, WO2017/025498, WO2017/149143, WO 2018/069500, WO2018/083087, WO2018/034227 WO2014/140180, the LAG-3 antagonistic protein AVA-017 (Avacta), the soluble LAG-3 fusion protein IMP321 (eftilagimod alpha; Immutep; see EP 2 205 257 and Brignone et al., 2007, J. Immunol., 179: 4202-4211), and soluble LAG-3 proteins disclosed in WO 2018/222711.

TIM-3 inhibitors include, without limitation, antibodies targeting TIM-3 such as F38-2E2 (BioLegend), cobolimab (TSR-022; Tesaro), LY3321367 (Eli Lilly), MBG453 (Novartis) and antibodies as disclosed in, e.g., WO 2013/006490, WO 2018/085469 (e.g., antibodies comprising heavy and light chain sequences encoded by nucleic acid sequences according to SEQ ID NOs: 3 and 4), WO 2018/106588, WO 2018/106529 (e.g., an antibody comprising heavy and light chain sequences according to SEQ ID NOs: 8-11).

TIM-3 ligand inhibitors include, without limitation, CEACAM1 inhibitors such as the anti-CEACAM1 antibody CM10 (cCAM Biotherapeutics; see WO 2013/054331), antibodies disclosed in WO 2015/075725 (e.g., CM-24, 26H7, 5F4, TEC-11, 12-140-4, 4/3/17, COL-4, F36-54, 34B1, YG-C28F2, D14HD11, M8.7.7, D11-AD11, HEA81, B I. I, CLB-gran-10, F34-187, T84.1, B6.2, B 1.13, YG-C94G7, 12-140-5, scFv DIATHIS1, TET-2; cCAM Biotherapeutics), antibodies described by Watt et al., 2001 (Blood, 98: 1469-1479) and in WO 2010/12557 and PtdSer inhibitors such as bavituximab (Peregrine).

CD94/NKG2A inhibitors include, without limitation, monalizumab (IPH2201; Innate Pharma) and the antibodies and method for their production as disclosed in U.S. Pat. No. 9,422,368 (e.g., humanized Z199; see EP 2 628 753), EP 3 193 929 and WO2016/032334 (e.g., humanized Z270; see EP 2 628 753).

IDO inhibitors include, without limitation, exiguamine A, epacadostat (INCB024360; InCyte; see U.S. Pat. No. 9,624,185), indoximod (Newlink Genetics; CAS #: 110117-83-4), NLG919 (Newlink Genetics/Genentech; CAS #: 1402836-58-1), GDC-0919 (Newlink Genetics/Genentech; CAS #: 1402836-58-1), F001287 (Flexus Biosciences/BMS; CAS #: 2221034-29-1), KHK2455 (Cheong et al., 2018, Expert Opin Ther Pat. 28(4):317-330), PF-06840003 (see WO 2016/181348), navoximod (RG6078, GDC-0919, NLG919; CAS #: 1402837-78-8), linrodostat (BMS-986205; Bristol-Myers Suibb; CAS #: 1923833-60-6), small molecules such as 1-methyl-tryptophan, pyrrolidine-2,5-dione derivatives (see WO 2015/173764) and the IDO inhibitors disclosed by Sheridan, 2015, Nat Biotechnol 33:321-322.

CD39 inhibitors include, without limitation, A001485 (Arcus Biosciences), PSB 069 (CAS #: 78510-31-3) and the anti-CD39 monoclonal antibody IPH5201 (Innate Pharma; see Perrot et al., 2019, Cell Reports 8:2411-2425.E9).

CD73 inhibitors include, without limitation, anti-CD73 antibodies such as CPI-006 (Corvus Pharmaceuticals), MEDI9447 (MedImmune; see WO2016075099), IPH5301 (Innate Pharma; see Perrot et al., 2019, Cell Reports 8:2411-2425.E9), the anti-CD73 antibodies described in WO2018/110555, the small molecule inhibitors PBS 12379 (Tocris Bioscience; CAS #: 1802226-78-3), A000830, A001190 and A001421 (Arcus Biosciences; see Becker et al., 2018, Cancer Research 78(13 Supplement):3691-3691, doi: 10.1158/1538-7445.AM2018-3691), CB-708 (Calithera Biosciences) and purine cytotoxic nucleoside analogue-based diphosphonates as described by Allard et al., 2018 (Immunol Rev., 276(1):121-144).

A2AR inhibitors include, without limitation, small molecule inhibitors such as istradefylline (KW-6002; CAS #: 155270-99-8), PBF-509 (Palobiopharma), ciforadenant (CPI-444: Corvus Pharma/Genentech; CAS #: 1202402-40-1), ST1535 ([2butyl-9-methyl-8-(2H-1,2,3-triazol 2-yl)-9H-purin-6-xylamine]; CAS #: 496955-42-1), ST4206 (see Stasi et al., 2015, Europ J Pharm 761:353-361; CAS #: 1246018-36-9), tozadenant (SYN115; CAS #: 870070-55-6), V81444 (see WO 2002/055082), preladenant (SCH420814; Merck; CAS #: 377727-87-2), vipadenant (BIIB014; CAS #: 442908-10-3), ST1535 (CAS #: 496955-42-1), SCH412348 (CAS #: 377727-26-9), SCH442416 (Axon 2283; Axon Medchem; CAS #: 316173-57-6), ZM241385 (4-(2-(7-amino-2-(2-furyl)-(1,2,4)triazolo(2,3-a)-(1,3,5)triazin-5-yl-amino)ethyl)phenol; Cas #: 139180-30-6), AZD4635 (AstraZeneca), AB928 (a dual A2AR/A2BR small molecule inhibitor; Arcus Biosciences) and SCH58261 (see Popoli et al., 2000, Neuropsychopharm 22:522-529; CAS #: 160098-96-4).

A2BR inhibitors include, without limitation, AB928 (a dual A2AR/A2BR small molecule inhibitor; Arcus Biosciences), MRS 1706 (CAS #: 264622-53-9), GS6201 (CAS #: 752222-83-6) and PBS 1115 (CAS #: 152529-79-8).

VISTA inhibitors include, without limitation, anti-VISTA antibodies such as JNJ-61610588 (onvatilimab; Janssen Biotech) and the small molecule inhibitor CA-170 (anti-PD-L1/L2 and anti-VISTA small molecule; CAS #: 1673534-76-3).

Siglec inhibitors include, without limitation, the anti-Sigle-7 antibodies disclosed in US 2019/023786 and WO 2018/027203 (e.g., an antibody comprising a variable heavy chain region according to SEQ ID NO: 1 and a variable light chain region according to SEQ ID NO: 15), the anti-Siglec-2 antibody inotuzumab ozogamicin (Besponsa; see U.S. Pat. No. 8,153,768 and U.S. Pat. No. 9,642,918), the anti-Siglec-3 antibody gemtuzumab ozogamicin (Mylotarg; see U.S. Pat. No. 9,359,442) or the anti-Siglec-9 antibodies disclosed in US 2019/062427, US 2019/023786, WO 2019/011855, WO 2019/011852 (e.g., an antibody comprising the CDRs according to SEQ ID NOs: 171-176, or 3 and 4, or 5 and 6, or 7 and 8, or 9 and 10, or 11 and 12, or 13 and 14, or 15 and 16, or 17 and 18, or 19 and 20, or 21 and 22, or 23 and 24, or 25 and 26), US 2017/306014 and EP 3 146 979.

CD20 inhibitors include, without limitation, anti-CD20 antibodies such as rituximab (RITUXAN; IDEC-102; IDEC-C2B8; see U.S. Pat. No. 5,843,439), ABP 798 (rituximab biosimilar), ofatumumab (2F2; see WO2004/035607), obinutuzumab, ocrelizumab (2h7; see WO 2004/056312), ibritumomab tiuxetan (Zevalin), tositumomab, ublituximab (LFB-R603; LFB Biotechnologies) and the antibodies disclosed in US 2018/0036306 (e.g., an antibody comprising light and heavy chains according to SEQ ID NOs: 1-3 and 4-6, or 7 and 8, or 9 and 10).

GARP inhibitors include, without limitation, anti-GARP antibodies such as ARGX-115 (arGEN-X) and the antibodies and methods for their production as disclosed in US 2019/127483, US 2019/016811, US 2018/327511, US 2016/251438, EP 3 253 796.

CD47 inhibitors include, without limitation, anti-CD47 antibodies such as HuF9-G4 (Stanford University/Forty Seven), CC-90002/INBRX-103 (Celgene/Inhibrx), SRF231 (Surface Oncology), IBI188 (Innovent Biologics), AO-176 (Arch Oncology), bispecific antibodies targeting CD47 including TG-1801 (NI-1701; bispecific monoclonal antibody targeting CD47 and CD19; Novimmune/TG Therapeutics) and NI-1801 (bispecific monoclonal antibody targeting CD47 and mesothelin; Novimmune), and CD47 fusion proteins such as ALX148 (ALX Oncology; see Kauder et al., 2019, PLoS One, doi: 10.1371/journal.pone.0201832).

SIRPα inhibitors include, without limitation, anti-SIRPα antibodies such as OSE-172 (Boehringer Ingelheim/OSE), FSI-189 (Forty Seven), anti-SIRPα fusion proteins such as TTI-621 and TTI-662 (Trillium Therapeutics; see WO 2014/094122).

PVRIG inhibitors include, without limitation, anti-PVRIG antibodies such as COM701 (CGEN-15029) and antibodies and method for their manufacture as disclosed in, e.g., WO 2018/033798 (e.g., CHA.7.518.1H4(S241P), CHA.7.538.1.2.H4(S241P), CPA.9.086H4(S241P), CPA.9.083H4(S241P), CHA.9.547.7.H4(S241P), CHA.9.547.13.H4(S241P) and antibodies comprising a variable heavy domain according to SEQ ID NO: 5 and a variable light domain according to SEQ ID NO: 10 of WO 2018/033798 or antibodies comprising a heavy chain according to SEQ ID NO:9 and a light chain according to SEQ ID NO: 14; WO 2018/033798 further discloses anti-TIGIT antibodies and combination therapies with anti-TIGIT and anti-PVRIG antibodies), WO2016134333, WO2018017864 (e.g., an antibody comprising a heavy chain according to SEQ ID NOs: 5-7 having at least 90% sequence identity to SEQ ID NO: 11 and/or a light chain according to SEQ ID NOs: 8-10 having at least 90% sequence identity to SEQ ID NO: 12, or an antibody encoded by SEQ ID NOs: 13 and/or 14 or SEQ ID NOs: 24 and/or 29, or another antibody disclosed in WO 2018/017864) and anti-PVRIG antibodies and fusion peptides as disclosed in WO 2016/134335.

CSF1R inhibitors include, without limitation, anti-CSF1R antibodies cabiralizumab (FPA008; FivePrime; see WO 2011/140249, WO 2013/169264 and WO 2014/036357), IMC-CS4 (EiiLilly), emactuzumab (R05509554; Roche), RG7155 (WO 2011/70024, WO 2011/107553, WO 2011/131407, WO 2013/87699, WO 2013/119716, WO 2013/132044) and the small molecule inhibitors BLZ945 (CAS #: 953769-46-5) and pexidartinib (PLX3397; Selleckchem; CAS #: 1029044-16-3).

CSF1 inhibitors include, without limitation, anti-CSF1 antibodies disclosed in EP 1 223 980 and Weir et al., 1996 (J Bone Mineral Res 11: 1474-1481), WO 2014/132072, and antisense DNA and RNA as disclosed in WO 2001/030381.

Exemplary NOX inhibitors include, without limitation, NOX1 inhibitors such as the small molecule ML171 (Gianni et al., 2010, ACS Chem Biol 5(10):981-93, NOS31 (Yamamoto et al., 2018, Biol Pharm Bull. 41(3):419-426), NOX2 inhibitors such as the small molecules ceplene (histamine dihydrochloride; CAS #: 56-92-8), BJ-1301 (Gautam et al., 2017, Mol Cancer Ther 16(10):2144-2156; CAS #: 1287234-48-3) and inhibitors described by Lu et al., 2017, Biochem Pharmacol 143:25-38, NOX4 inhibitors such as the small molecule inhibitors VAS2870 (Altenhöfer et al., 2012, Cell Mol Life Sciences 69(14):2327-2343), diphenylene iodonium (CAS #: 244-54-2) and GKT137831 (CAS #: 1218942-37-0; see Tang et al., 2018, 19(10):578-585). TDO inhibitors include, without limitation, 4-(indol-3-yl)-pyrazole derivatives (see U.S. Pat. No. 9,126,984 and US 2016/0263087), 3-indol substituted derivatives (see WO 2015/140717, WO 2017/025868, WO 2016/147144), 3-(indol-3-yl)-pyridine derivatives (see US 2015/0225367 and WO 2015/121812), dual IDO/TDO antagonist, such as small molecule dual IDO/TDO inhibitors disclosed in WO 2015/150097, WO 2015/082499, WO 2016/026772, WO 2016/071283, WO 2016/071293, WO 2017/007700, and the small molecule inhibitor CB548 (Kim, C, et al., 2018, Annals Oncol 29 (suppl_8): viii400-viii441).

According to the disclosure, the immune checkpoint inhibitor is an inhibitor of an inhibitory checkpoint protein but preferably not an inhibitor of a stimulatory checkpoint protein. As described herein, a number of CTLA-4, PD-1, TIGIT, B7-H3, B7-H4, BTLA, KIR, LAG-3, TIM-3, CD94/NKG2A, IDO, A2AR, A2BR, VISTA, Siglec, CD20, CD39, CD73, GARP, CD47, PVRIG, CSF1R, NOX and TDO inhibitors and inhibitors of respective ligands are known and several of them are already in clinical trials or even approved. Based on these known immune checkpoint inhibitors, alternative immune checkpoint inhibitors may be developed. In particular, known inhibitors of the preferred immune checkpoint proteins may be used as such or analogues thereof may be used, in particular chimerized, humanized or human forms of antibodies and antibodies cross-competing with any of the antibodies described herein.

It will be understood by one of ordinary skill in the art that other immune checkpoint targets can also be targeted by antagonists or antibodies, provided that the targeting results in the stimulation of an immune response such as an anti-tumor immune response as reflected in an increase in T cell proliferation, enhanced T cell activation, and/or increased cytokine production (e.g., IFN-γ, IL2).

Checkpoint inhibitors may be administered in any manner and by any route known in the art. The mode and route of administration will depend on the type of checkpoint inhibitor to be used.

Checkpoint inhibitors may be administered in the form of any suitable pharmaceutical composition as described herein.

Checkpoint inhibitors may be administered in the form of nucleic acid, such DNA or RNA molecules, encoding an immune checkpoint inhibitor, e.g., an inhibitory nucleic acid molecule or an antibody or fragment thereof. For example, antibodies can be delivered encoded in expression vectors, as described herein. Nucleic acid molecules can be delivered as such, e.g., in the form of a plasmid or mRNA molecule, or complexed with a delivery vehicle, e.g., a liposome, lipoplex or nucleic-acid lipid particles. Checkpoint inhibitors may also be administered via an oncolytic virus comprising an expression cassette encoding the checkpoint inhibitor. Checkpoint inhibitors may also be administered by administration of endogeneic or allogeneic cells able to express a checkpoint inhibitor, e.g., in the form of a cell based therapy. The term “cell based therapy” refers to the transplantation of cells (e.g., T lymphocytes, dendritic cells, or stem cells) expressing an immune checkpoint inhibitor into a subject for the purpose of treating a disease or disorder (e.g., a cancer disease). In one embodiment, the cell based therapy comprises genetically engineered cells. In one embodiment, the genetically engineered cells express an immune checkpoint inhibitor, such as described herein. In one embodiment, the genetically engineered cells express an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. Genetically engineered cells may also express further agents that enhance T cell function. Such agents are known in the art. Cell based therapies for the use in inhibition of immune checkpoint signaling are disclosed, e.g., in WO 2018/222711, herein incorporated by reference in its entirety.

The term “oncolytic virus” as used herein, refers to a virus capable of selectively replicating in and slowing the growth or inducing the death of a cancerous or hyperproliferative cell, either in vitro or in vivo, while having no or minimal effect on normal cells. An oncolytic virus for the delivery of an immune checkpoint inhibitor comprises an expression cassette that may encode an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. The oncolytic virus preferably is replication competent and the expression cassette is under the control of a viral promoter, e.g., synthetic early/late poxvirus promoter. Exemplary oncolytic viruses include vesicular stomatitis virus (VSV), rhabdoviruses (e.g., picornaviruses such as Seneca Valley virus; SVV-001), coxsackievirus, parvovirus, Newcastle disease virus (NDV), herpes simplex virus (HSV; OncoVEX GMCSF), retroviruses (e.g., influenza viruses), measles virus, reovirus, Sinbis virus, vaccinia virus, as exemplarily described in WO 2017/209053 (including Copenhagen, Western Reserve, Wyeth strains), and adenovirus (e.g., Delta-24, Delta-24-RGD, ICOVIR-5, ICOVIR-7, Onyx-015, ColoAd1, H101, AD5/3-D24-GMCSF). Generation of recombinant oncolytic viruses comprising a soluble form of an immune checkpoint inhibitor and methods for their use are disclosed in WO 2018/022831, herein incorporated by reference in its entirety. Oncolytic viruses can be used as attenuated viruses.

### Radiotherapy

Radiotherapy (RT) is the second most common treatment regimen used and given to approximately 50% of cancer patients. Different types of RT treatments exist, using high energy photons (X- and γ-ray), particle irradiation (e.g. protons, carbon ions) or radionuclides (cesium 137, iridium 192, iodine 125) to locally deliver radiation to malignant tissues. Local RT (LRT) faces an over 120 year-long history of technological improvements and radiation dose-refinement.

Ionizing radiation refers to radiation that has enough energy to ionize matter and can be divided into electromagnetic radiation (X- and γ-ray) and particulate radiation (α and β particles, protons, heavy ions). A natural source of ionizing radiation are radioisotopes that, upon radioactive decay, emit a unique spectrum of α (He²⁺), β (e⁻ or e⁺) and γ rays (high energy photons). The number of ionization events and tissue penetration depth is a function of the primary kinetic energy and, if charged, coulomb energy. The radiation dose is measured in gray (Gy) as the International Systems of Units (SI) unit for the absorbed radiation dose per unit mass of matter (1 J radiation/kg matter).

When ionizing radiation traverses biological matter, it is able to break chemical bonds and ionize molecules, with direct and severe effects on cells, tissues and the organism as a whole. Whereas all cellular structures may be harmed by radiation directly or indirectly (indirect ionization by radiation-induced reactive oxygen species (ROS)), DNA damage is regarded as the ultimate and most severe consequence. Ionizing radiation may cause different types of DNA damage, such as base damage, single-strand breaks (SSB) and double-strand breaks (DSB). The type and density of DNA damage is a function of radiation type (e.g. electromagnetic or particulate) and dose. The exact cellular fate, however, depends on cell-intrinsic factors such as cell type, cell cycle phase, repair capability and cell death-proficiency as a function of the DNA-damage response (DDR) initiated. First, if the cell is repair-proficient and the damage minimal, the cell may repair its damage. Base damages and SSB are repaired via base-excision repair (BER) or nucleotide excision repair (NER), whereas more severe DSB are repaired via non-homologous end-joining (NHEJ, all cell cycle phases) or homologous recombination (HR, only S and G2 phase). Second, the cell may try to repair the DNA damage, but fails to succeed and reproduces in its damaged form. Whereas unrepaired DSB can lead to cell death by mitotic catastrophe, misrepaired DSB give rise to chromosomal translocation and genomic instability and can cause secondary cancers. Third, the cell recognizes the damage and undergoes programmed cell death (apoptosis).

As most cancer cells exhibit aberrant DNA repair pathways and impaired cell cycle control, they might respond to ionizing radiation differently than their healthy counterparts.

The linear-quadratic (LQ) model of cell killing is one of the key mathematical tools in radiation biology and physics, and provides a simple relationship between delivered dose and cell survival. Different radiation doses are applied in vitro and a cell's survival determined by its ability to produce a viable colony of progenitor cells, measured in a clonogenic survival assay. Clonogenic survival assays are the accepted gold standard to measure a cells survival in response to radiation, and is defined by S=e^{-αD-βD}^{²}, with S being the surviving cell fraction, D the total dose, and α and β the measure of a cell's radiosensitivity. The surviving fraction is plotted on a log scale against the radiation dose. The survival curve follows a linear slope (α) at low doses and a curved slope (β) at higher doses. The early and late bent of this curve is a cell-intrinsic characteristic and expressed as the α/β-ratio. Cells with a high α/β-ratio experience a relatively constant rate of cell death across different doses, whereas cells with a low α/β-ratio show a pronounced curvature and respond to high dose radiation with increased cell death. Slowly proliferating cells or tissues, such as most healthy cells, generally repair very well and have a low α/β-ratio (late responding tissues). Rapidly proliferating cells or tissues, such as tumor cells, generally repair worse and have a high α/β-ratio (acute responding tissues). Especially at low doses (<2-2.5 Gy), normal cells have a survival advantage over tumor cells due to slower growth and intact DNA repair. The higher radiosensitivity of tumor cells at low doses forms the basis of fractionated radiotherapy, which is still used in standard clinical practice today.

However, there are serious limitations of the LQ model to predict ionizing radiation effects: (i) It is an in vitro model used to predict in vivo radiation effects, (ii) it measures clonogenic survival, however no information is provided about the type of cell death induced, e.g. mitotic catastrophe, apoptosis, necrosis, necroptosis, autophagy or replicative senescence, (iii) it does not account for the reaction of the immune system and (iv) is not accurate at higher doses per fraction (>10 Gy).

For the radiotherapeutic treatment of tumors, two types of radiation machines are utilized: electromagnetic and particulate machines. Depending on the type (electromagnetic or particulate) and primary energy, radiation beams possess different dose-deposition profiles into tissues.

Whereas photons are not able to deeply penetrate tissues and, depending on their primary energy, deposit their energy within the first 5 to 10 cm of water, particulate beams like proton beams are able to deeper penetrate tissues. X-rays are most commonly used in conventional radiotherapy as they are relatively inexpensive, less deleterious as particulate irradiation and therefore considerate safer.

The therapeutic use of X-rays faces a long history of technological developments with many of the early radiation machines still being used today. The development of orthovoltage X-ray tubes (200-500 kilovolt (kV)) in the 1930s first allowed the external treatment of tumors with X-ray beams. Contrary to natural X- or γ-ray emitting radionuclides, an X-ray tube is a vacuum tube that generates X-rays from electrical input. Electrons are emitted from a cathode and accelerated through the vacuum towards an anode. Depending on the tube voltage (50 kV to 500 kV), electrons are accelerated to different speeds and X-rays of different energy produced. When electrons collide with the anode material, bremsstrahlung in the X-range is produced perpendicular to the electron beam. In contrast to naturally emitting radionuclides, radiation is only produced as long as the X-ray tube is turned on. The resulting X-ray energy is a function of tube voltage and anode material. Due to their low tissue penetration depth, orthovoltage X-rays are abandoned in clinical practice, yet they remain frequently applied in preclinical research.

Today, megavoltage X-rays (1 to 25 MeV) are in use, utilizing medical linear accelerators (LINAC) to generate high energy X-rays for therapeutic purposes. Size, shape and angle of the beam are controlled to cover the tumor while spearing healthy tissues. Conventional, 3D conformal RT (3D-CRT) and intensity modulated RT (IMRT) are different forms of external beam RT (EBRT). In conventional RT, the radiation dose is delivered from different angles in multiple overlapping beams. The highest dose is delivered at the beam intersection within the tumor and doses decline with distance from the intersection point. In 3D-CRT, a tumor 3D image (computed tomography (CT), magnetic resonance imaging (MRI) or positron emission tomography (PET)) is used to design radiation beams that are more conform to the shape of the tumor, and more accurately outline organs at risk. IMRT is an advanced form of 3D-CRT, in which the beam is divided in hundreds of beamlets of different intensities, enabling a highly conformal dose distribution and highly precise tumor targeting.

In contrast to electromagnetic irradiation, particle irradiation was introduced for therapeutic use in the 1970s, using proton or carbon beams. Particle irradiation is characterized by its good penetration potential and high energy deposition at the end of its range (Bragg peak). This allows extremely steep dose gradients with limited dose-deposition along the trajectory. Radiation machines are, however, very cost intensive and high dose-deposition is accompanied by a high risk of secondary cancers.

At times when radiation delivery and radiation machines were rudimentary, total radiation doses were fractionated to multiple smaller ones, not because of the appreciation of the underlying biology, but because of technical limitations and the desire to reduce off-target effects. Today, clinical LRT protocols are still based on fractionated LRT, applying daily 1.8 to 2 Gy (Monday to Friday) for six to eight weeks, accumulating total doses of 60 to 80 Gy. Due to technological advances in radiation delivery, radiation doses can be delivered at high accuracy, reduced margins and high dose conformation. This allows the delivery of higher radiation doses in a single fraction at reduced risk, which is referred to as stereotactic body RT (SBRT). In addition, immunomodulatory effects of LRT became known, especially when high doses per fraction are applied. Due to the favorable immunological effects, a change is underway, increasingly applying high dose LRT alone or in conjunction with other immunomodulatory agents.

### Pharmaceutical Compositions

The agents described herein may be administered in pharmaceutical compositions or medicaments and may be administered in the form of any suitable pharmaceutical composition.

A pharmaceutical composition may comprise a pharmaceutically acceptable carrier and may optionally comprise one or more adjuvants, stabilizers etc. In one embodiment, a pharmaceutical composition is for therapeutic or prophylactic treatments, e.g., for use in treating or preventing cancer.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The cytokines may be IL1, IL2, IL3, IL4, IL5, IL6, IL7, IL8, IL9, IL10, IL12, IFNα, IFNγ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease.

The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

In some embodiments, an effective amount comprises an amount sufficient to cause a tumor/lesion to shrink. In some embodiments, an effective amount is an amount sufficient to decrease the growth rate of a tumor (such as to suppress tumor growth). In some embodiments, an effective amount is an amount sufficient to delay tumor development. In some embodiments, an effective amount is an amount sufficient to prevent or delay tumor recurrence. In some embodiments, an effective amount is an amount sufficient to increase a subject's immune response to a tumor, such that tumor growth and/or size and/or metastasis is reduced, delayed, ameliorated, and/or prevented. An effective amount can be administered in one or more administrations. In some embodiments, administration of an effective amount (e.g., of a composition comprising mRNAs) may: (i) reduce the number of cancer cells; (ii) reduce tumor size; (iii) inhibit, retard, slow to some extent and may stop cancer cell infiltration into peripheral organs; (iv) inhibit (e.g., slow to some extent and/or block or prevent) metastasis; (v) inhibit tumor growth; (vi) prevent or delay occurrence and/or recurrence of tumor; and/or (vii) relieve to some extent one or more of the symptoms associated with the cancer.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient.

Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical composition is formulated for systemic administration, e.g., for intravenous administration.

The term “co-administering” as used herein means a process whereby different compounds or compositions (e.g., RNA encoding antigenic epitopes and RNA encoding immunostimulant) are administered to the same patient. The different compounds or compositions may be administered simultaneously, at essentially the same time, or sequentially.

### Treatments

The present invention provides methods and agents for inducing an immune response, in particular for inducing an immune response against a target antigen or cells expressing a target antigen, e.g., tumor cells expressing a target antigen, in a subject comprising administering an effective amount of RNA encoding immunostimulant and optionally RNA encoding antigenic epitopes.

In one embodiment, the methods and agents described herein provide immunity in a subject to a disease or disorder associated with a target antigen. The present invention thus provides methods and agents for treating or preventing the disease, or disorder associated with the target antigen.

In one embodiment, the methods and agents described herein are administered to a subject having a disease, or disorder associated with a target antigen. In one embodiment, the methods and agents described herein are administered to a subject at risk for developing the disease, or disorder associated with the target antigen.

The therapeutic compounds or compositions of the invention may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In some embodiments, administration of a composition of the present invention may be performed by single administration or boosted by multiple administrations.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “malaise” relates to a feeling of general discomfort, uneasiness, or pain, often the first sign of an infection or other disease.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide an immune response against cancer cells, and to treat a cancer disease. In one embodiment, the cancer is an antigen-positive cancer.

Pharmaceutical compositions described herein are applicable for inducing or enhancing an immune response. Pharmaceutical compositions described herein are thus useful in a prophylactic and/or therapeutic treatment of a disease involving an antigen or epitope.

As used herein, “immune response” refers to an integrated bodily response to an antigen or a cell expressing an antigen and refers to a cellular immune response and/or a humoral immune response. The immune system is divided into a more primitive innate immune system, and acquired or adaptive immune system of vertebrates, each of which contains humoral and cellular components.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to immune effector cells, in particular to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as cancer cells, preventing the production of more diseased cells.

The term “effector functions” in the context of the present invention includes any functions mediated by components of the immune system that result, for example, in the killing of diseased cells such as cancer cells. In one embodiment, the effector functions in the context of the present invention are T cell mediated effector functions. Such functions comprise in the case of a helper T cell (CD4⁺ T cell) the release of cytokines and/or the activation of CD8⁺ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-α, and specific cytolytic killing of antigen expressing target cells.

The term “immune effector cell” or “immunoreactive cell” in the context of the present invention relates to a cell which exerts effector functions during an immune reaction. An “immune effector cell” in one embodiment is capable of binding an antigen such as an antigen presented in the context of MHC on a cell or expressed on the surface of a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4⁺ and/or CD8⁺ T cells, most preferably CD8⁺ T cells. According to the invention, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system. Upon activation, cytotoxic lymphocytes trigger the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells.

A “lymphoid cell” is a cell which is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell.

The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell”, “T cell specific for an antigen” or similar terms relate to a T cell which recognizes the antigen to which the T cell is targeted, in particular when presented on the surface of antigen presenting cells or diseased cells such as cancer cells in the context of MHC molecules, and preferably exerts effector functions of T cells.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptor (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4+ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8+ T cells since they express the CD8 glycoprotein on their surface. These cells recognize their targets by binding to antigen associated with MHC class I, which is present on the surface of nearly every cell of the body. A majority of T cells have a T cell receptor (TCR) existing as a complex of several proteins. The TCR of a T cell is able to interact with immunogenic peptides (epitopes) bound to major histocompatibility complex (MHC) molecules and presented on the surface of target cells. Specific binding of the TCR triggers a signal cascade inside the T cell leading to proliferation and differentiation into a maturated effector T cell. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

“Humoral immunity” or “humoral immune response” is the aspect of immunity that is mediated by macromolecules found in extracellular fluids such as secreted antibodies, complement proteins, and certain antimicrobial peptides. It contrasts with cell-mediated immunity. Its aspects involving antibodies are often called antibody-mediated immunity.

The present disclosure contemplates an immune response that may be protective, preventive, prophylactic and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”. The term “immunotherapy” relates to the treatment of a disease or condition by inducing, or enhancing an immune response. The term “immunotherapy” includes antigen immunization or antigen vaccination and immunostimulation.

The terms “immunization” or “vaccination” describe the process of administering an antigen to an individual with the purpose of inducing an immune response, for example, for therapeutic or prophylactic reasons.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In one embodiment, the macrophages are splenic macrophages.

The term “dendritic cell” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naïve T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen” refers to any disease which implicates an antigen, e.g. a disease which is characterized by the presence of an antigen. The disease involving an antigen can be cancer. As mentioned above, the antigen may be a disease-associated antigen, such as a tumor antigen. In one embodiment, a disease involving an antigen is a disease involving cells expressing an antigen.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma.

The term “cancer” according to the disclosure also comprises cancer metastases.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1. Preparation of RiboCytokine RNA Lipid Nanoparticles and RNA Vaccines

RiboCytokine mRNA was generated by in vitro transcription based on Kreiter et al. (Kreiter, S. et al. Cancer Immunol. Immunother. 56, 1577-87 (2007)) with substitution of the nucleoside uridine by N1-methyl-pseudouridine. Resulting mRNAs were equipped with a Cap1-structure and double-stranded (dsRNA) molecules were depleted by cellulose purification (Baiersdörfer et al., Mol. Ther. (2019)). Purified mRNA was eluted in H₂O and stored at −80° C. until further use. In vitro transcription of all described mRNA constructs was carried out at BioNTech RNA Pharmaceuticals GmbH. The modified RNA was encapsulated within lipid nanoparticles (LNPs) that mediate preferential delivery of the RNA to the liver after i.v. administration. RNA for vaccination was produced based on Kreiter et al. (Kreiter, S. et al. Cancer Immunol. Immunother. 56, 1577-87 (2007)) using a Beta-S-ARCA(D1) cap. RNA-LPX formulation was performed based on Kranz et al., Nature (2016).

### Example 2. Administration of hAlb-hIL2_A4s8 but not hAlb-hIL2 Leads to Toxicity in Mice

We aimed to assess the potential adverse effects of hAlb-hIL2_A4s8 administration with two rational combination partners, a RiboCytokine encoding IL-7 (hIL7-hAlb), and an antigen-encoding T-cell vaccine (RNA-lipoplex (RNA-LPX)). To this end, naïve C57BL/6 mice were treated i.v. with either the triple combination of 5 μg RNA-LPX, 3 μg hAlb-hIL2_A4s8, and 3 μg hIL7-hAlb (n=7); 3 μg hAlb-hIL2_A4s8 and 3 μg hIL7-hAlb (n=7); 5 μg RNA-LPX and 3 μg hAlb-hIL2_A4s8 (n=7); or 5 μg RNA-LPX, 3 μg hAlb-hIL2, and 3 μg hIL7-hAlb (n=7). NaCl-treated animals served as negative control (n=7).

Parameters monitored in the experiment included body weight as well as the activity of ALAT, ASAT, and LDH. Animal weight was recorded on day 2 and day 3 after treatment. Body mass changes were calculated as the ratio of the current weight of each animal to its body weight before treatment (day 0). All enzymes were analyzed on day 3 as surrogates for liver and tissue damage. For the analysis of enzyme levels, blood samples were collected from the vena facialis according to standard procedures. After collection of blood in serum separator tubes, the samples were allowed to clot for approximately 30 minutes before centrifugation. Immediately after centrifugation, the serum was frozen and stored. Before measurement, serum was thawed at ambient temperature, diluted with water, and enzyme activity was determined using the Indiko™ Clinical Chemistry Analyzer (Thermo Fisher Scientific), according to the manufacturer's protocol.

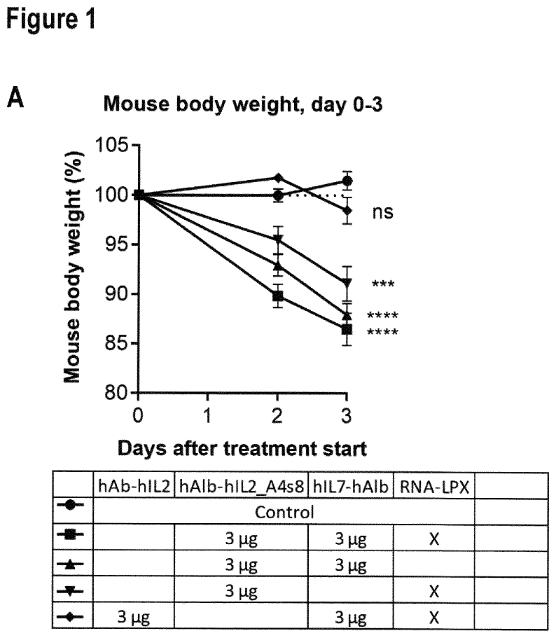

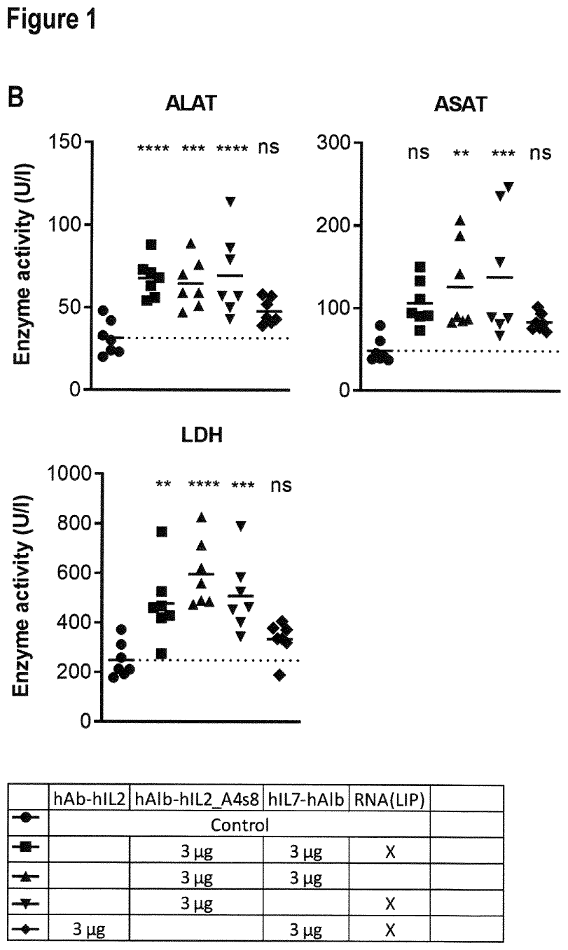

Administration of mice with 3 μg hAlb-hIL2_A4s8, but not 3 μg hAlb-hIL2, in combination with hIL7-hAlb and RNA-LPX vaccination resulted in a significant weight loss of more than 10% within three days after treatment (FIG. 1A). A slightly reduced weight loss was observed in groups receiving hAlb-hIL2_A4s8 together with only hIL7-hAlb or RNA-LPX, indicating that both combination partners contribute to the overall toxicity. Similarly, a significant increase in the activity of liver enzymes ALAT, ASAT, and LDH was solely detected in groups receiving hAlb-hIL2_A4s8 but not hAlb-hIL2 (FIG. 1B).

### Example 3. hAlb-hIL2_A4s8 Triggers a Temporary Increase in NK Cells Only after the First Treatment

One of the potential mediators of unwanted effects described in Example 1 was hypothesized to be NK cells. Activated NK cells were shown to secrete large amounts of IFNγ responsible for the immunotoxicity induced by an IL15-superagonist (Guo et al. J. Immunol. 2015). To this end, we studied NK cell dynamics in CT26 colon carcinoma-bearing mice. hIL7-hAlb, hAlb-hIL2, as well as hAlb-hIL2_A4s8 were assessed together with RNA-LPX vaccination against the CT26 tumor-specific antigen gp70.

BALB/c mice were subcutaneously (s.c.) inoculated with 5×10⁵ CT26 colon carcinoma cells on day 0. Ten days later, mice were stratified according to tumor volume into four treatment groups of 11 mice each. Treatment consisted of four weekly injections of i.v. administered RiboCytokines (hAlb-hIL2, IL7-hAlb, or hAlb-hIL2_A4s8; 3 μg each) and concomitant 20 μg gp70 RNA-LPX vaccine on day 10, 17, 24, and 31. The control group received 6 μg hAlb-encoding RNA.

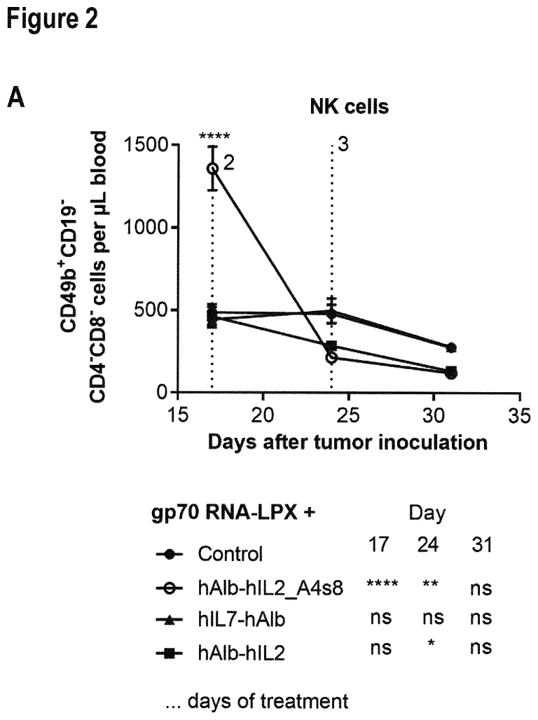

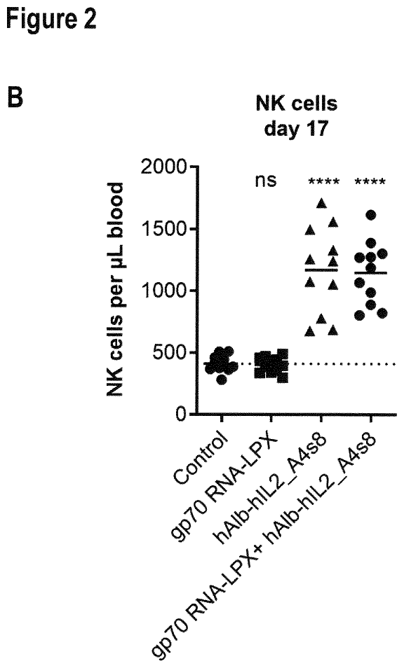

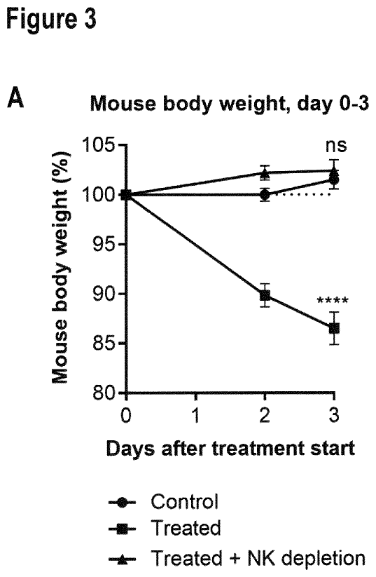

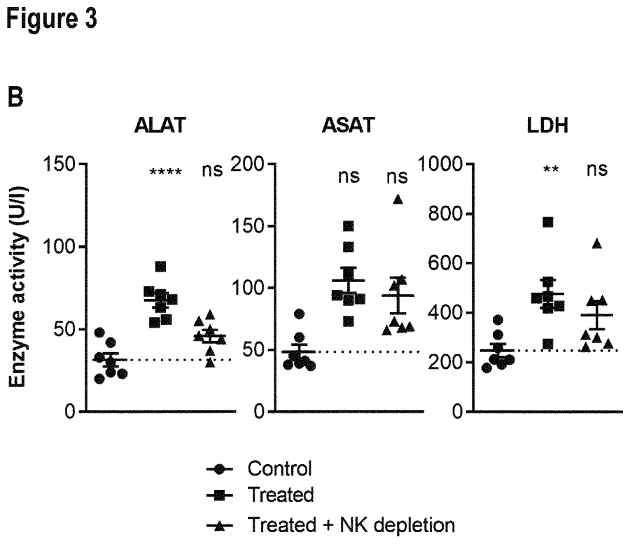

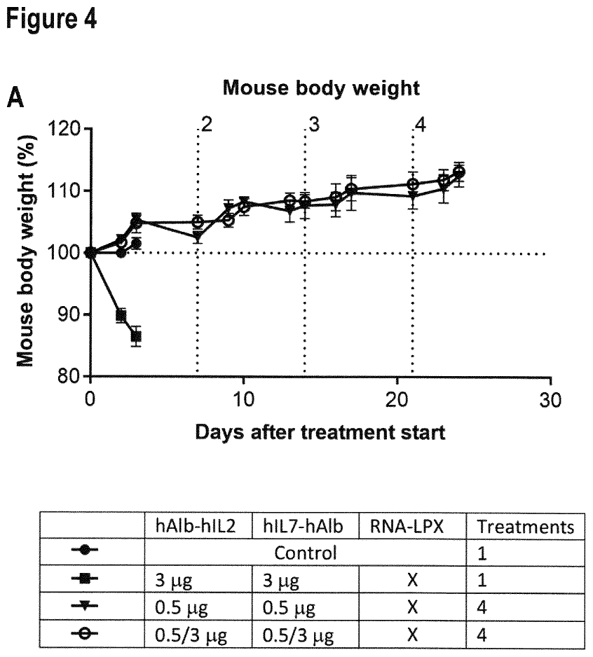

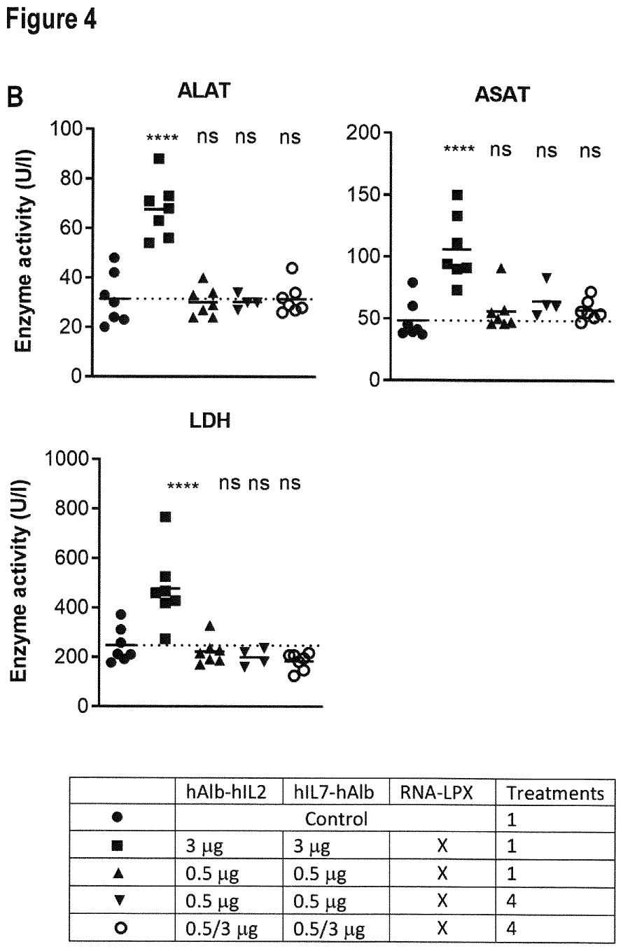

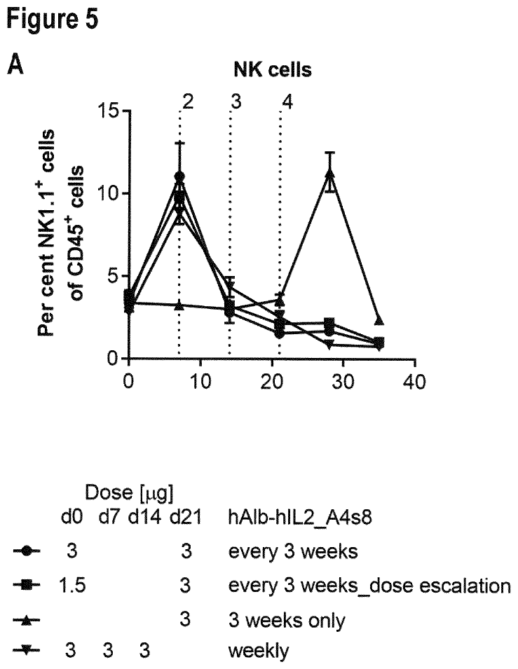

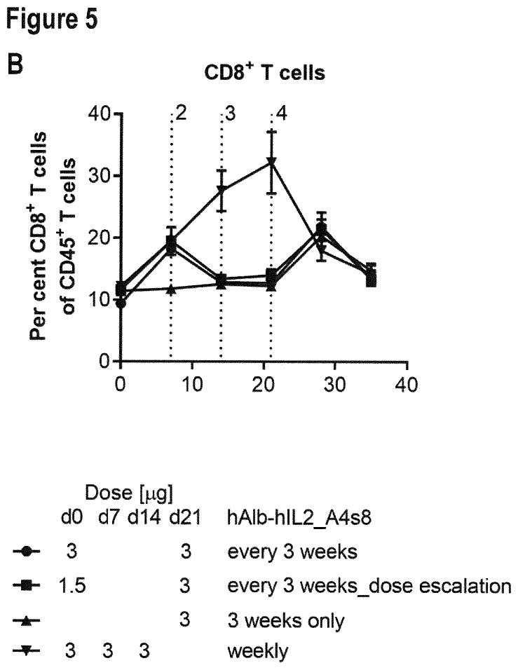

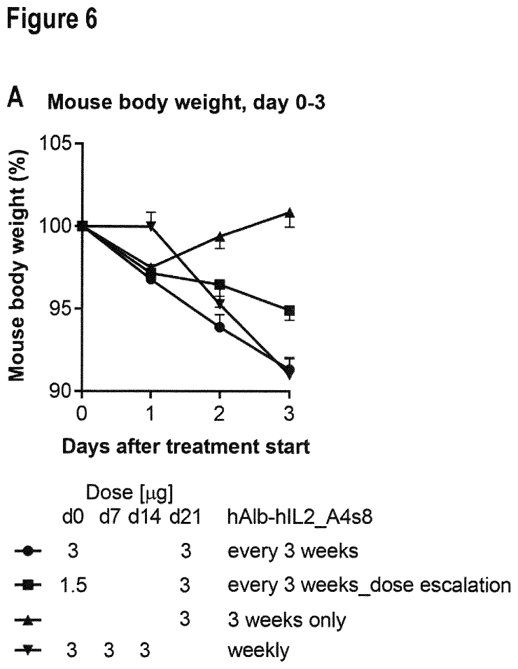

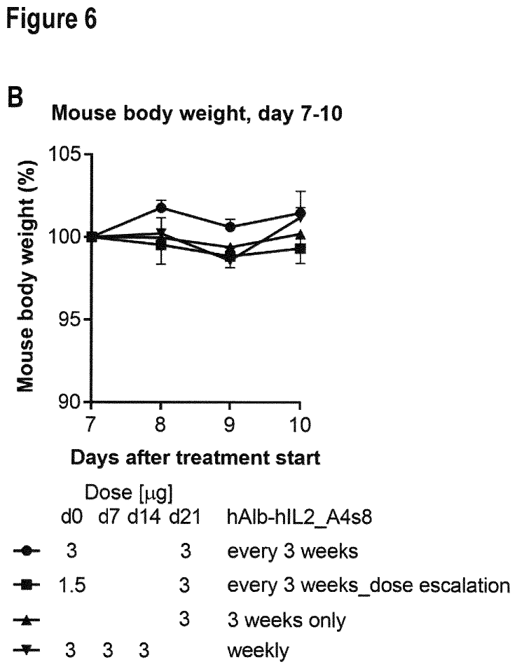

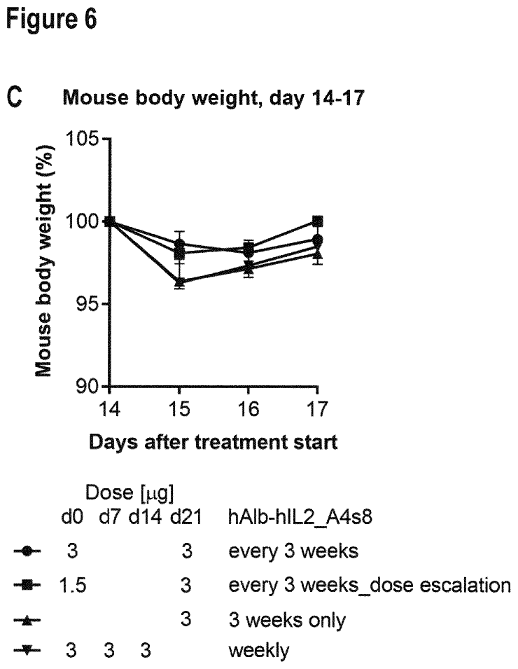

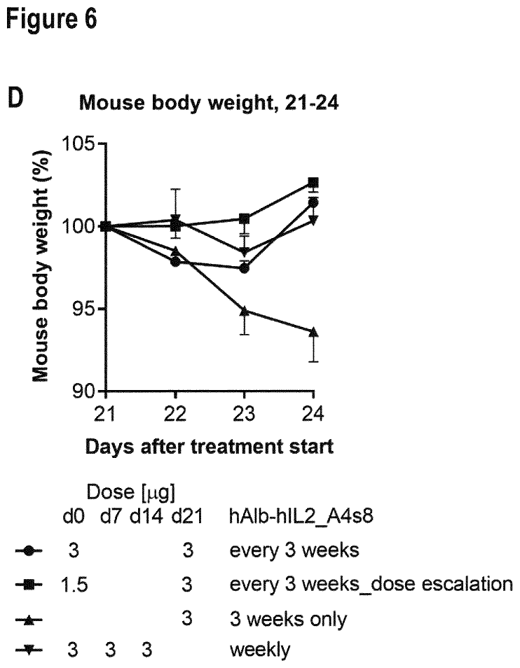

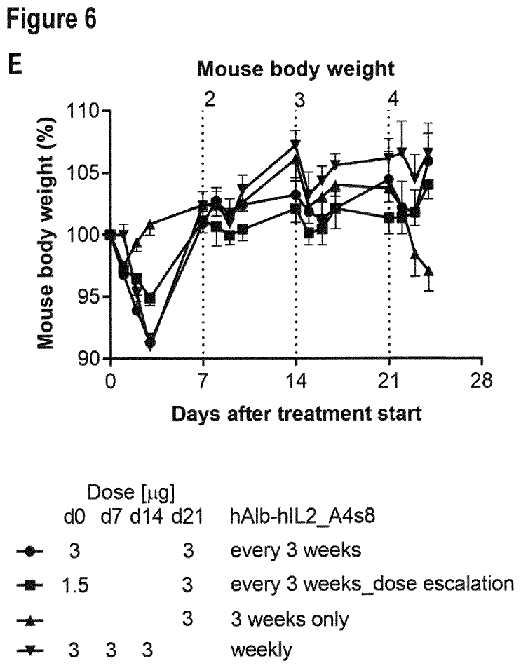

The numbers of CD49b⁺CD19⁻CD4⁻CD8⁻ NK cells in the blood of treated animals were analyzed by flow cytometry on day 17, 24, and 31. In brief, 50 μL of blood were stained with titrated amounts of antibodies, and erythrocytes were lysed using BD lysing solution subsequently. Cells were washed with PBS, resuspended, and transferred to a Trucount tube (BD Biosciences). Data were acquired on a BD FACSCelesta™ Flow Cytometer (BD Biosciences) and analyzed with FlowJo software version 10.3 and GraphPad Prism version 8.4. hAlb-hIL2_A4s8 treatment resulted in a temporary 2.8-fold increase of NK cells one week after first dosing (FIG. 2A). Numbers normalized within 14 days after the initial treatment and were not elevated by subsequent hAlb-hIL2_A4s8 administrations on days 17 and 24. Neither hAlb-hIL2 nor IL7-hAlb injection resulted in notable NK cell expansion.

To address the potential contribution of the RNA vaccine to NK cell expansion, BALB/c mice were inoculated s.c. with 5×10⁵ CT26 colon carcinoma cells on day 0. Ten days later, mice were stratified according to tumor volume into four treatment groups of 11 mice each and treated i.v. with 20 μg gp70 RNA-LPX, 3 μg LNP-formulated RNA encoding hAlb-hIL2_A4s8 or the combination of both. The control group received RNA-LPX (not coding for any antigen) plus 3 μg LNP-formulated hAlb-encoding RNA. NK cell numbers were assessed in peripheral blood as described above.

Irrespective of gp70-RNA-LPX vaccination, hAlb-hIL2_A4s8 treatment resulted in significant 2.8-fold increase of NK cells seven days after treatment (day 17 after tumor inoculation; FIG. 2B). No change in cell numbers was observed after gp70-RNA-LPX injection alone. These results suggest that hAlb-hIL2_A4s8 itself, and not the RNA-LPX vaccine, is responsible for the observed NK cell expansion.

Collectively, these findings highlight the capacity of hAlb-hIL2_A4s8 to drive NK cell expansion after the first dosing, which is followed by rapid contraction and unresponsiveness to subsequent treatments.

### Example 4. hAlb-hIL2_A4s8-Associated Toxicity is Dependent on NK Cells

In order to determine if NK cells accounted for the toxicity observed in hAlb-hIL2_A4s8-treated mice (Example 1), we compared the body weight kinetics and serum liver enzyme activity in mice treated on days 0, 7, 14 and 21 with 3 μg hAlb-hIL2_A4s8, 3 μg hIL7-hAlb, and RNA-LPX, with or without NK cell depletion via intraperitoneal administration of 20 μL anti-asialo GM1 antibody one day before treatment initiation (n=7 per group).

NK cell depletion prior to treatment start prevented the weight loss (FIG. 3A) and precluded the elevation in liver enzyme activity observed in the group that had not received the NK cell-depleting antibody after the first dosing (day 3; FIG. 3B).

These data directly link NK cells to the toxic effects of hAlb-hIL2_A4s8 treatment.

### Example 5. Low-Dose hAlb-hIL2_A4s8 Preconditioning Increases Treatment Tolerability

Toxic side effects of hAlb-hIL2_A4s8 treatment were shown to be dependent on a temporary expansion of NK cells (Example 4). Importantly, NK cells were able to expand only after the first hAlb-hIL2_A4s8 injection and remained refractory thereafter (Example 3). Based on these findings, we hypothesized that NK cell activation would be feasible with lower, less toxic doses and that an initial low dose of hAlb-hIL2_A4s8 would improve the tolerability of subsequent higher doses.

C57BL/6 mice (n=7 per group) were treated i.v. either with NaCl (control); 5 μg RNA-LPX vaccine, 3 μg hAlb-hIL2_A4s8 and 3 μg hIL7-hAlb on day 0 (3 μg), or 5 μg RNA-LPX vaccine, 0.5 μg hAlb-hIL2_A4s8 and 0.5 μg hIL7-hAlb on day 0, 7, 14 and 21 (0.5 μg). Treatment tolerability of the 3 μg or 0.5 μg cytokine dose was compared to groups that received a preconditioning treatment on day 0 with 5 ag RNA-LPX, 0.5 μg hIL7-hAlb and 0.5 μg hAlb-hIL2_A4s8, and were treated with 5 μg RNA-LPX and 3 μg of each cytokine on days 7, 14 and 21 (0.5/3 μg). Body weight as well as liver enzyme activity in the serum were determined as described in Example 2.

We found that initial low-dose preconditioning alleviated the weight loss (FIG. 4A) as well as the increase in liver enzyme activity in the serum (FIG. 4B) of subsequent high cytokine doses (0.5/3 ag group). Animals treated directly with high-dose RiboCytokines (3 μg group) exhibited a weight loss of more than 14% within the first three days of treatment initiation as well as a significant increase in liver enzyme activity. In comparison, a weight gain similar to control animals was observed for mice that received only 0.5 μg of cytokines (0.5 ag group). Similarly, liver enzymes after one or after four doses of 0.5 μg cytokine remained in the range of control animals.

These data highlight the robust treatment tolerability in animals that received an initial low dose of RiboCytokines, regardless of whether the animals subsequently received higher RiboCytokine doses or were maintained on a low-dose RiboCytokine regimen.

### Example 6. Low Dose Preconditioning Increases the Tolerability of hAlb-hIL2_A4s8 Even in a Three Week Dosing Regime

All experiments shown so far assessed weekly RiboCytokine administration. In clinical settings, however, it is likely that RiboCytokines will be administered at a more relaxed frequency such as every three weeks. In theory, an increased time between individual treatments could allow for repetitive NK cell expansions and associated toxicity. Accordingly, we tested whether a preconditioning regimen would increase the tolerability of hAlb-hIL2_A4s8 even if administered as relaxed as every three weeks.

C57BL/6 mice (n=5 per group) received 3 μg hAlb-hIL2_A4s8 either at day 0, 7 and 14 (weekly), on day 0 and 21 (every 3 weeks), or at day 21 only (3 weeks only). The preconditioning regiment consisted of a 1.5 μg hAlb-hIL2_A4s8 dose on day 0 followed by 3 μg hAlb-hIL2_A4s8 on day 21 (every 3 weeks dose escalation). NK cell and CD8*T cell frequencies were determined by flow cytometry in blood samples collected on days 0, 7, 14, 21, 28, and 35 as described in Example 3. To evaluate hAlb-hIL2_A4s8 treatment tolerability, animal weight was assessed for three consecutive days after each hAlb-hIL2_A4s8 injection.

NK cell frequencies increased by more than three-fold in all groups of treated mice seven days after the first hAlb-hIL2_A4s8 injection (day 7) (FIG. 5A). By day 14, the NK cell frequencies dropped back to baseline levels and remained refractory, regardless of the treatment schedule. The frequencies of CD8⁺ T cells also significantly increased seven days after the first hAlb-hIL2_A4s8 administration, reaching similar values in all treated groups (FIG. 5B). In the three-week schedules, this was followed by contraction to baseline levels by day 14 and subsequent similar expansion kinetics upon second dosing. These data indicated that after a three-week interval, CD8⁺ T cells still had the same capacity to expand as upon the initial RiboCytokine injection and, unlike NK cells, did not appear refractory to repeated hAlb-hIL2_A4s8 treatment.

Treatment with hAlb-hIL2_A4s8 resulted in a dose dependent weight loss from 5% (1.5 μg) up to 9% (3 μg) within three days after initial treatment (FIG. 6A). Subsequent weekly treatments were well tolerated (FIG. 6B, C). As expected from the NK cell expansion profile (FIG. 5A), toxicity from day 21 onwards was observed only in mice that received their first 3 μg dose of hAlb-hIL2_A4s8 at this point, but not in mice that had been preconditioned with 1.5 μg three weeks earlier (FIG. 6D, E).

To summarize, in the current disclosure, we identified well-tolerated RiboCytokine treatment regimens of potential clinical relevance for any type of immunotherapy that leads to an expansion of NK cells (e.g. IL-15 and its variants, as well as type-I interferon inducers). These treatment schedules provide an important platform for the clinical development of safe and efficacious immunotherapies that can meet the urgent medical needs of cancer patients.

1. A method of reducing an unwanted response or reaction, or both, in a subject, to RNA encoding an amino acid sequence comprising a cytokine protein, said method comprising administering to a subject:
a first dose of said RNA;
a second dose of said RNA; and
wherein the dosages and time periods of administration of said first and second doses are selected such that the level of unwanted response or reaction is reduced in said subject, optionally wherein said subject is a human subject.

2. The method of claim 1, wherein the amount of said RNA administered in said first dose is no more than 80%, 75%, 50%, 40%, 30%, 25%, 20%, 15%, 10% or 5% of the amount of said RNA administered in said second dose, or
wherein the amount of said RNA administered in said first dose is no more than 200 μg, 150 μg, 100 μg, 90 μg, 80 μg, 70 μg, 60 μg, 50 μg, 40 μg, 30 μg, 20 μg, 10 μg, 5 μg, 4 μg, 3 μg, 2 μg, 1 μg, 0.5 μg, 0.4 μg, 0.3 μg, 0.2 μg, or 0.1 μg per kg body weight, and the second dose is greater than said first dose, or
wherein the amount of said RNA administered in said second dose is greater than 20 μg, 30 μg, 40 μg, 50 μg, 60 μg, 70 μg, 80 μg, 90 μg, 100 μg, 150 μg, 200 μg 250 μg, 300 μg, 350 μg, or 400 μg per kg body weight, and the second dose is greater than said first dose.

3-4. (canceled)

5. The method of claim 1, wherein more than 1, 2, 3, 4, 5, 6, 7, 14, or 21 days separate the completion of the administration of the first dose and the initiation of the administration of the second dose, or
wherein no more than 56, 49, 42, 35, or 28 days separates the completion of the administration of the first dose and the initiation of the administration of the second dose.

6. (canceled)

7. The method of claim 1, further comprising administering to the subject one or more additional doses of RNA encoding an amino acid sequence comprising a cytokine protein.

8. The method of claim 1, wherein said first and second doses are administered by intravenous, intraarterial, subcutaneous, intraperitoneal, intradermal or intramuscular injection or infusion.

9. (canceled)

10. The method of claim 1, wherein the unwanted response or reaction involves NK cells, or
wherein the unwanted response or reaction comprises one or more selected from the group consisting of increase in NK cell number, fever, malaise, reduction of body weight, increase in activity of liver enzymes, capillary leak syndrome, hypotension and edema, preferably wherein the liver enzymes comprise one or more selected from the group consisting of alanine-aminotransferase (ALAT), aspartate-aminotransferase (ASAT), and lactate-dehydrogenase (LDH), or
wherein the unwanted response or reaction occurs after administration of the second dose without administration of the first dose.

11-13. (canceled)

14. The method of claim 1, further comprising evaluating the subject after administration of the first dose, the second dose, or both, for the presence of an unwanted response or reaction.

15. The method of claim 1, wherein said method does not cause a detectable unwanted response or reaction, or
wherein said method results in a decrease in unwanted response or reaction.

16. (canceled)

17. The method of claim 1, which further comprises administering a vaccine to the subject.

18. The method of claim 17, wherein administering a vaccine to the subject comprises administering to the subject RNA encoding one or more antigenic epitopes, optionally wherein the epitopes are T cell epitopes.

19. (canceled)

20. The method of claim 1, wherein the amino acid sequence comprising a cytokine protein comprises an extended pharmacokinetic (PK) polypeptide, optionally wherein the wherein the extended PK polypeptide comprises a fusion protein, further optionally wherein the fusion protein comprises the cytokine protein fused to a pharmacokinetic modifying group.

21-22. (canceled)

23. The method of claim 20, wherein the pharmacokinetic modifying group comprises albumin, a functional variant thereof, or a functional fragment of the albumin or the functional variant thereof, or
wherein the pharmacokinetic modifying group comprises human albumin, a functional variant thereof, or a functional fragment of the human albumin or the functional variant thereof, or wherein the pharmacokinetic modifying group is fused to the N-terminus of the cytokine protein.

24-25. (canceled)

26. The method of claim 1, wherein the amino acid sequence comprising a cytokine protein comprises from N-terminus to C-terminus: N-pharmacokinetic modifying group-GS-linker-cytokine protein-C.

27. The method of claim 1, wherein the cytokine protein comprises an IL2 variant, optionally wherein the IL2 variant is a human IL2 variant, optionally wherein the human IL2 variant comprises a substitution variant of human IL2 or of a functional variant of human IL2, optionally wherein human IL2 has the amino acid sequence according to SEQ ID NO: 1.

28-29. (canceled)

30. The method of claim 27, wherein the substitution(s) enhance(s) the affinity for the βγ IL2 receptor complex (IL2Rβγ).

31. The method of claim 27, wherein the human IL2 or the functional variant thereof is substituted at at least position 80 (leucine), position 81 (arginine), position 85 (leucine) and position 92 (isoleucine) relative to wild type human IL2 and numbered in accordance with wild type human IL2, optionally wherein position 80 (leucine) is substituted by phenylalanine, position 81 (arginine) is substituted by glutamic acid, position 85 (leucine) is substituted by valine and position 92 (isoleucine) is substituted by phenylalanine relative to wild type human IL2 and numbered in accordance with wild type human IL2, or
wherein the human IL2 or the functional variant thereof is further substituted at position 74 (glutamine) relative to wild type human IL2 and numbered in accordance with wild type human IL2, optionally wherein position 74 (glutamine) is substituted by histidine relative to wild type human IL2 and numbered in accordance with wild type human IL2.

32-34. (canceled)

35. The method of claim 27, wherein the substitution(s) reduce(s) the affinity for the alpha subunit of the αβγ IL2 receptor complex (IL2Rαβγ), optionally wherein the substitution(s) which reduce(s) the affinity for the alpha subunit of the αβγ IL2 receptor complex (IL2Rαβγ) reduce the affinity for IL2Rαβγ to a greater extent than for IL2Rβγ.

36. (canceled)

37. The method of claim 27, wherein the human IL2 or the functional variant thereof is substituted at at least position 43 (lysine) and position 61 (glutamic acid) relative to wild type human IL2 and numbered in accordance with wild type human IL2, optionally wherein position 43 (lysine) is substituted by glutamic acid and position 61 (glutamic acid) is substituted by lysine.

38. (canceled)

39. The method of claim 27, wherein the IL2 variant has a decreased ability to stimulate regulatory T cells compared to wild type human IL2, or wherein the IL2 variant has an increased ability to stimulate effector T cells compared to wild type human IL2.

40. (canceled)

41. The method of claim 1, wherein the cytokine protein comprises a mutein of human IL2 or of a functional variant of human IL2, wherein the human IL2 or the functional variant thereof is substituted at least position 43 (lysine) by glutamic acid, position 61 (glutamic acid) by lysine, position 74 (glutamine) by histidine, position 80 (leucine) by phenylalanine, position 81 (arginine) by glutamic acid, position 85 (leucine) by valine and position 92 (isoleucine) by phenylalanine relative to wild type human IL2 and numbered in accordance with wild type human IL2.

42. (canceled)

43. The method of claim 1, wherein the amino acid sequence comprising a cytokine protein comprises the amino acid sequence according to SEQ ID NO: 6 (hAlb-hIL2_A4s8).

44. (canceled)

45. A kit comprising:
a first dose of RNA encoding an amino acid sequence comprising a cytokine protein; and
a second dose of RNA encoding an amino acid sequence comprising a cytokine protein; and
wherein the dosages of said first and second doses are selected such that upon administration of said first and second doses to a subject the level of unwanted response or reaction is reduced in said subject, optionally wherein the composition and/or amounts of the RNA of the first dose and/or the RNA of the second dose is as defined in any one of the foregoing claims.

46. (canceled)

47. The kit of claim 45, wherein the RNA of the first dose and the RNA of the second dose are in separate vials.

48. (canceled)
